(19) United States
(12) Patent Application Publication
(10) Pub. No.: US 2026/0263640 A1
(43) Pub. Date: Sep. 10, 2026

(54) GENE THERAPY COMPOSITIONS, METHODS, AND SYSTEMS FOR WEIGHT LOSS AND MAINTENANCE

(71) Applicant: Fractyl Health, Inc., Burlington, MA (US)

(72) Inventors: Michael A. Biasella, Burlington, MA (US); Jay Caplan, Burlington, MA (US); Douglas Garrity, Burlington, MA (US); Sara A. Morneau, Burlington, MA (US); Shimyn Slomovic, Burlington, MA (US); Jacob Wainer, Burlington, MA (US); Suya Wang, Burlington, MA (US); Gary White, Burlington, MA (US); Lin Quek, Burlington, MA (US)

(73) Assignee: Fractyl Health, Inc., Burlington, MA (US)

(21) Appl. No.: 19/561,151

(22) Filed: Mar. 9, 2026

Related U.S. Application Data
(60) Provisional application No. 63/769,671, filed on Mar. 10, 2025.
(60) Provisional application No. 63/786,919, filed on Apr. 10, 2025.

(51) Int. Cl. A61K 48/00; A61K 9/00; A61K 38/00; A61M 5/158; A61P 3/10; C07K 14/005; C07K 14/605; C12N 15/86

(52) U.S. Cl. CPC A61K 48/0058; A61K 9/0019; A61K 48/0075; A61K 48/0083; A61M 5/158; A61P 3/10; C07K 14/005; C07K 14/605; C12N 15/86; A61K 38/00; A61M 2005/1586; C07K 2319/02; C12N 2750/14122; C12N 2750/14143

(57) ABSTRACT

The technology relates to pancreatic gene therapy compositions and methods for weight loss and maintenance, including the prevention of weight gain. Infusion systems for infusing the gene therapy compositions are also described.

### RELATED APPLICATIONS

This application claims the benefit under 35 U.S.C. § 119 (e) to U.S. Provisional Application No. 63/769,671, filed Mar. 10, 2025, and to U.S. Provisional Application No. 63/786,919, filed Apr. 10, 2025, the contents of each of which are incorporated herein by reference in their entirety.

### REFERENCE TO AN ELECTRONIC SEQUENCE LISTING

The contents of the electronic sequence listing (F085770041WO00-SEQ-HJD.xml; Size: 31,481 bytes; and Date of Creation: Feb. 17, 2026) are herein incorporated by reference in their entirety.

### BACKGROUND

Obesity and overweight conditions pose significant public health challenges worldwide, contributing to an increased risk of metabolic disorders, cardiovascular diseases, and other chronic conditions. While various treatments, including dietary modifications, pharmacological interventions, and surgical procedures, have been developed to facilitate weight loss, sustained weight maintenance remains a critical challenge. For example, it has been difficult to halt progressive weight gain and control blood glucose levels. Studies indicate that a substantial percentage of individuals who lose weight eventually regain it, often exceeding their initial weight.

### SUMMARY

The present technology, in some aspects, relates to nutrient responsive, adaptive pancreatic gene therapy compositions and methods for weight loss and for preventing weight gain, for example, when challenged with lifestyle changes (e.g., dietary changes and/or physical changes). The present technology, in other aspects, relates to nutrient responsive, adaptive pancreatic gene therapy compositions and methods for regulating blood sugar, and in some embodiments, treating or preventing type 2 diabetes and/or hyperglycemia. Surprisingly, the pancreatic gene therapy compositions provided herein can be used not only to reduce weight in an overweight individual but also to prevent weight gain (e.g., after losing weight) without the individual becoming sarcopenic or hypoglycemic, even when switched to a normal or low-calorie diet.

Some aspects of the technology relate to a method for preventing weight gain in a subject, comprising: injecting pancreatic tissue of the subject with an effective amount of a viral vector, such as an adeno-associated virus (AAV) vector, comprising (i) a recombinant viral (e.g., AAV) genome comprising a pancreatic islet cell specific promoter operably linked to a transgene comprising a GLP-1 receptor agonist coding sequence and (ii) viral capsid proteins (e.g., AAV capsid proteins, such as AAV9 capsid proteins), wherein the subject has a body mass index (BMI) of less than 30 kg/m². In some embodiments, a subject has a BMI of less than 25 kg/m². In some embodiments, a subject has a BMI of 18.5-24.9 kg/m².

In some embodiments, an effective amount of a pancreatic gene therapy composition for preventing weight gain is sufficient for a subject to maintain a BMI of less than 30 kg/m² on a high caloric diet (e.g., a high fat diet) for an extended period, for example, at least 7 days. In some embodiments, an effective amount is sufficient for a subject to maintain a BMI of less than 30 kg/m² on a high caloric diet for at least 14 days. In some embodiments, an effective amount is sufficient for a subject to maintain a BMI of less than 30 kg/m² on a high caloric diet for at least 21 days. In some embodiments, an effective amount is sufficient for a subject to maintain a BMI of less than 30 kg/m² on a high caloric diet for at least 28 days. Extended periods on the order of months and years are also contemplated herein.

Other aspects provide a method for treating obesity in a subject, comprising: injecting pancreatic tissue of the subject with an effective amount of an AAV vector comprising (i) a recombinant AAV genome comprising a pancreatic islet cell specific promoter operably linked to a transgene comprising a GLP-1 receptor agonist coding sequence and (ii) AAV (e.g., AAV9) capsid proteins, wherein the subject has a BMI of at least 30 kg/m².

In some embodiments, an effective amount of a pancreatic gene therapy composition for treating obesity is sufficient to maintain a clinically significant reduction in body weight (e.g., at least 5% of starting body weight) for an extended period, for example, at least 3 months. In some embodiments, an effective amount is sufficient to maintain a clinically significant reduction in body weight (e.g., at least 5% of starting body weight) for at least 6 months. In some embodiments, an effective amount is sufficient to maintain a clinically significant reduction in body weight (e.g., at least 5% of starting body weight) for at least 9 months. In some embodiments, an effective amount is sufficient to maintain a clinically significant reduction in body weight for at least 1 year (e.g., at least 5% of starting body weight). A “clinically significant reduction in body weight” includes a reduction of at least 5% relative to a starting weight, which is the weight of a subject at the time a pancreatic gene therapy composition is administered.

In some embodiments, an effective amount of a pancreatic gene therapy composition for treating obesity is sufficient for the subject to maintain a BMI of less than 30 kg/m² (or less than 25 kg/m²) on a high caloric diet (e.g., a high fat diet) for at 3 months. In some embodiments, an effective amount is sufficient for the subject to maintain a BMI of less than 30 kg/m² (or less than 25 kg/m²) on a high caloric diet for at 6 months. In some embodiments, an effective amount is sufficient for the subject to maintain a BMI of less than 30 kg/m² (or less than 25 kg/m²) on a high caloric diet for at 9 months. In some embodiments, an effective amount is sufficient for the subject to maintain a BMI of less than 30 kg/m² (or less than 25 kg/m²) on a high caloric diet for at 1 year.

Yet other aspects relate to a method for treating type 2 diabetes in a subject, comprising: injecting pancreatic tissue of the subject with an effective amount of a viral (e.g., an AAV) vector comprising (i) a recombinant viral (e.g., AAV) genome comprising a pancreatic islet cell specific promoter operably linked to a transgene comprising a GLP-1 receptor agonist coding sequence and (ii) viral capsid proteins (e.g., AAV capsid proteins, such as AAV9 capsid proteins), wherein the subject has type 2 diabetes. In some embodiments, a subject having type 2 diabetes has a BMI of at least 25 kg/m². In some embodiments, a subject having type 2 diabetes has a BMI of at least 30 kg/m².

In some embodiments, an effective amount of a pancreatic gene therapy composition for treating type 2 diabetes is sufficient to lower fasting blood glucose in a subject to below 126 mg/dL. In some embodiments, an effective amount is sufficient to lower fasting blood glucose in a subject to below 100 mg/dL. In some embodiments, an effective amount of a pancreatic gene therapy composition for treating type 2 diabetes is sufficient to lower fasting blood glucose in a subject by at least 5% relative to a starting fasting blood glucose.

In some embodiments, an effective amount of a pancreatic gene therapy composition for treating type 2 diabetes is sufficient for a subject to maintain a BMI of less than 30 kg/m² for at least 14 days. In some embodiments, an effective amount is sufficient for the subject to maintain a BMI of less than 30 kg/m² for at least 21 days. In some embodiments, an effective amount is sufficient for the subject to maintain a BMI of less than 30 kg/m² for at least 28 days. Extended periods on the order of months and years are also contemplated herein.

Still other aspects of the technology relate to a method for regulating blood glucose levels in a subject, comprising: injecting pancreatic tissue of the subject with an effective amount of an AAV vector comprising (i) a recombinant AAV genome comprising a pancreatic islet cell specific promoter operably linked to a transgene comprising a GLP-1 receptor agonist coding sequence and (ii) AAV (e.g., AAV9) capsid proteins, wherein the subject has a fasting blood glucose of at least 100 mg/dL. In some embodiments, a subject has a fasting blood glucose of at least 126 mg/dL.

In some embodiments, an effective amount is sufficient to treat hyperglycemia in a subject (e.g., to maintain fasting blood glucose below 126 mg/dL, or prevent fasting blood glucose from rising to 126 mg/dL or above).

In some embodiments, a recombinant AAV genome comprises (e.g., in the following 5′ to 3′ order): a first AAV inverted terminal repeat (ITR) sequence, a pancreatic islet cell specific promoter, a transgene, a polyadenylation (polyA) sequence, and a second AAV ITR.

In some embodiments, a GLP-1 receptor agonist coding sequence is a GLP-1 (1-37) peptide coding sequence. In other embodiments, a GLP-1 receptor agonist coding sequence is a GLP-1 (1-36) peptide coding sequence.

In some embodiments, a first AAV inverted terminal repeat (ITR) sequence comprises the sequence of SEQ ID NO: 11, a pancreatic islet cell specific promoter comprises the sequence of SEQ ID NO: 6, a GLP-1 receptor agonist coding sequence of a transgene comprises the sequence of SEQ ID NO: 7, a WPRE comprises the sequence of SEQ ID NO: 9, a polyadenylation (polyA) sequence comprising the sequence of SEQ ID NO: 10, and a second AAV ITR comprising the sequence of SEQ ID NO: 12.

In some embodiments, a pancreatic islet cell specific promoter comprises a human insulin promoter sequence. In some embodiments, a human insulin promoter sequence comprises the sequence of SEQ ID NO: 6. In some embodiments, a human insulin promoter sequence comprises a sequence that has at least 90%, at least 95%, or at least 98% identity to the sequence of SEQ ID NO: 6.

In some embodiments, a transgene further comprises a human insulin signal peptide, for example, upstream from the GLP-1 receptor agonist coding sequence. In some embodiments, a human insulin signal peptide comprises the sequence of SEQ ID NO: 13. In some embodiments, a human insulin signal peptide comprises a sequence that has at least 90%, at least 95%, or at least 98% identity to the sequence of SEQ ID NO: 13.

In some embodiments, a transgene further comprises an intervening peptide 2 (IP2) peptide coding sequence downstream from the GLP-1 receptor agonist coding sequence. In some embodiments, an IP2 peptide coding sequence comprises the sequence of SEQ ID NO: 8. In some embodiments, an IP2 peptide coding sequence comprises a sequence that has at least 90%, at least 95%, or at least 98% identity to the sequence of SEQ ID NO: 8.

In some embodiments, a transgene comprises the sequence of SEQ ID NO: 1. In some embodiments, a transgene comprises a sequence that has at least 90%, at least 95%, or at least 98% identity to the sequence of SEQ ID NO: 1.

In some embodiments, a recombinant AAV genome further comprises a Woodchuck Hepatitis Virus Posttranscriptional Regulatory Element (WPRE) sequence, for example, downstream from the transgene and upstream from the polyA sequence. In some embodiments, a WPRE sequence comprises the sequence of SEQ ID NO: 9. In some embodiments, a WPRE sequence comprises a sequence that has at least 90%, at least 95%, or at least 98% identity to the sequence of SEQ ID NO: 9.

In some embodiments, a recombinant AAV genome comprises the sequence of SEQ ID NO: 2. In some embodiments, a recombinant AAV genome comprises a sequence that has at least 90%, at least 95%, or at least 98% identity to the sequence of SEQ ID NO: 2. In some embodiments, a recombinant AAV genome comprises the sequence of SEQ ID NO: 3. In some embodiments, a recombinant AAV genome comprises a sequence that has at least 90%, at least 95%, or at least 98% identity to the sequence of SEQ ID NO: 3.

In some embodiments, an effective amount is about 5×10¹² to about 5×10¹⁴ AAV vector genomes (VG). As is known in the art, a “vector genome” (i.e., “VG”) amount refers to the number of genomic copies of a viral (or non-viral) vector used in gene therapy.

In some embodiments, injecting of a pancreatic tissue is via endoscopic ultrasound-guided fine needle injection (EUS-FNI). In some embodiments, injecting of the pancreatic tissue comprises a single infusion. In some embodiments, injection of the pancreatic tissue comprises multiple infusions. In some embodiments, injection of the pancreatic tissue comprises at least three infusions.

In some embodiments, the pancreatic tissue is in a tail region of the pancreas. In some embodiments, the pancreatic tissue is in a body region of the pancreas.

Some aspects of the technology relate to a polynucleotide comprising, in the 5′ to 3′ direction: a first AAV inverted terminal repeat (ITR) sequence comprising the sequence of SEQ ID NO: 11, a human insulin promoter comprising the sequence of SEQ ID NO: 6, a human insulin signal peptide coding sequence comprising the sequence of SEQ ID NO: 13, a GLP-1 coding sequence comprising the sequence of SEQ ID NO: 7, an IP2 coding sequence comprising the sequence of SEQ ID NO: 8, a WPRE sequence comprising the sequence of SEQ ID NO: 9, a polyA signal sequence comprising the sequence of SEQ ID NO: 10, and a second AAV ITR sequence comprising the sequence of SEQ ID NO: 12. In some embodiments, a polynucleotide comprises the nucleotide sequence of SEQ ID NO: 1. In some embodiments, a polynucleotide comprising the nucleotide sequence of SEQ ID NO: 2. In some embodiments, a polynucleotide comprising the nucleotide sequence of SEQ ID NO: 3.

Other aspects of the technology relate to an AAV viral vector comprising a polynucleotide of any of preceding paragraph and AAV (e.g., AAV9) capsid proteins.

Yet other aspects of the technology relate to an AAV viral vector comprising a recombinant AAV genome comprising the sequence of SEQ ID NO: 3 and AAV (e.g., AAV9) capsid proteins.

Still other aspects of the technology relate to a plasmid comprising a polynucleotide of any of preceding paragraph.

Some aspects of the technology relate to a plasmid comprising (e.g., in the 5′ to 3′ direction): a first AAV inverted terminal repeat (ITR) sequence, a human insulin promoter, a human insulin signal peptide coding sequence, a GLP-1 coding sequence, an IP2 coding sequence, a WPRE sequence, a polyA signal sequence, and a second AAV ITR sequence.

Other aspects of the technology relate to a plasmid comprising the sequence of SEQ ID NO: 4. In some embodiments, a plasmid comprises a sequence having at least 90%, at least 95%, or at least 98% identity to the sequence of SEQ ID NO: 4.

### BRIEF DESCRIPTION OF THE DRAWINGS

FIGS. 1A-1B show body weight preceding and following substitution of a regular low-fat diet (Chow) for a high-fat diet (HFD) in mice treated with either vehicle (Veh) or an adeno-associated virus (AAV) encoding a glucagon-like peptide-1 (GLP-1) receptor agonist. Untreated mice maintained on a regular diet, or mice maintained on a high-fat diet treated with an AAV encoding a GLP-1 receptor agonist, were used as controls. FIG. 1A shows body weight in grams over time. FIG. 1B shows percent change in body weight over time.

FIG. 2 shows 4-hour fasting blood glucose levels preceding and following substitution of a low-fat diet for a high-fat diet in mice treated with either vehicle or an AAV encoding a GLP-1 receptor agonist. Untreated mice maintained on a regular diet, or mice maintained on a high-fat diet treated with an AAV encoding a GLP-1 receptor agonist, were used as controls.

FIGS. 3A-3B show food intake (kcals) prior to substitution of a low-fat diet for a high-fat diet in mice treated with either vehicle or an AAV encoding a GLP-1 receptor agonist. Untreated mice maintained on a regular diet, or mice maintained on a high-fat diet treated with an AAV encoding a GLP-1 receptor agonist, were used as controls. FIG. 3A shows daily food intake. FIG. 3B shows cumulative food intake over time.

FIGS. 4A-4B show food intake (grams) following substitution of a low-fat diet for a high-fat diet in mice treated with either vehicle or an AAV encoding a GLP-1 receptor agonist. Untreated mice maintained on a regular diet, or mice maintained on a high-fat diet treated with an AAV encoding a GLP-1 receptor agonist, were used as controls. FIG. 4A shows daily food intake. FIG. 4B shows cumulative food intake over time.

FIGS. 5A-5B show body mass (grams) prior to substitution of a low-fat diet for a high-fat diet in mice treated with either vehicle or an AAV encoding a GLP-1 receptor agonist. Untreated mice maintained on a regular diet, or mice maintained on a high-fat diet treated with an AAV encoding a GLP-1 receptor agonist, were used as controls. FIG. 5A shows body mass 16 days following initial treatment. FIG. 5B shows change in body mass from baseline 16 days following initial treatment.

FIGS. 6A-6B show body mass after substitution of a low-fat diet for a high-fat diet in mice treated with either vehicle or an AAV encoding a GLP-1 receptor agonist. Untreated mice maintained on a regular diet, or mice placed on a high-fat diet following treatment with an AAV encoding a GLP-1 receptor agonist, were used as controls. FIG. 6A shows body mass 31 days following initial treatment. FIG. 6B shows change in body mass from baseline 31 days following initial treatment.

FIG. 7 illustrates a partial view of a non-limiting example of an endoscope and infusion system for infusing a gene therapy composition into the pancreas.

FIG. 8 is a plot of puncture force measurements for different needles.

FIG. 9 illustrates an example of such an infusion system, including a handle, lumen, and needle.

FIGS. 10A-10C are views of an example handheld infusion system according to aspects of the present disclosure.

FIG. 11A is a view of an example fluid stop mechanism of an inner lumen assembly.

FIG. 11B is view of the distal end of the inner lumen assembly of FIG. 11A.

FIG. 11C is a view of the inner fluid lumen assembly showing the connection between a distal needle and a fluid lumen.

FIG. 11D is a cross-sectional view of the connection of FIG. 11C.

FIG. 11E is a detailed view of the distal tip of the distal needle of FIG. 11B.

FIG. 11F shows that the need may include a hyperechogenic coating in at least some embodiments to facilitate visualization during ultrasound imaging.

FIG. 12 is a partially transparent view of the fluid stop mechanism of FIG. 11A.

FIG. 13A is a view of a proximal end of an example outer sheath assembly.

FIG. 13B is a view of the distal end of the outer sheath assembly of FIG. 13A.

FIG. 13C is a cross-sectional view of the proximal end of the outer sheath assembly of FIG. 13A.

FIG. 14A is a cross-sectional view of the distal end of the inner fluid lumen assembly of FIG. 11B with the distal end of the outer sheath assembly of FIG. 13B in an operational configuration.

FIG. 14B is a view of the assembly of FIG. 14A.

FIGS. 15A-15D are views of the handle portion of the example handheld infusion system showing the installation of a medicament syringe.

FIG. 16 shows a drive mechanism of the handle portion of the example handheld infusion system engaged with a plunger of an installed medicament syringe prior in a ready-to-use configuration.

FIGS. 17A and 17B are views of the activation button and safety stop mechanism of the of the handle portion of the example handheld infusion system.

FIG. 18A is a cross-sectional view of the handle portion showing the drive mechanism in an intermediate stop position after a first infusion.

FIG. 18B is a view of the handle portion showing the drive mechanism in the intermediate stop position.

FIG. 19 is a view of the handle portion showing the drive mechanism in a final position after a second infusion.

FIG. 20 is a view of the actuation mechanism of the handle portion showing a carrier of the drive mechanism in an initial position.

FIG. 21 is a view of the example handheld infusion system with the body of the handle portion removed from view to show the relationship between the inner lumen assembly and the outer sheath assembly.

FIG. 22 is a view of the proximal end of the body portion showing the fixation of the thrust nut of the inner fluid assembly to the body of the handle portion.

FIG. 23 is a view of the distal end of the body portion with the sheath adjustment slider removed to show the magnetic attachment element on the proximal end of the body and the friction roller inside the body.

FIGS. 24A and 24B show, respectively, the handle portion and the distal end of the assembly in a position with the needle retracted.

FIGS. 24C and 24D show, respectively, the handle portion and the distal end of the assembly in a position with the needle extended.

FIGS. 25A-25C illustrate a photograph of the distal portion of a shaft including a hyperechogenic coating, and examples of uncoated and coated needles as viewed using ultrasonic imaging, respectively, consistent with the present inventive concepts.

FIG. 26 illustrates a sectional view of an embodiment of an infusion assembly, consistent with the present inventive concepts.

FIG. 27 illustrates an anatomic view of an embodiment of an infusion device inserted into tissue, consistent with the present inventive concepts.

FIG. 28 shows a schematic of the INS.GLP1 viral vector genome.

FIGS. 29A-29K show the targeted, effective, and safe delivery of AAV9.INS.GLP1 via EUS-guided pancreatic infusion in Yucatan pigs. FIG. 29A shows vector copy number (VCN) in pigs treated with the AAV9.INS.GLP1 high dose (4.04E13 vg) presented as vector genomes per diploid genome (VG/DG) measured in pancreas splenic lobe biopsies numbered according to the Key (below). FIG. 29B shows adjusted transgene RNA copies per 10 ng total RNA input in pancreatic splenic lobe biopsies. FIG. 29C shows active GLP-1 levels (pg per μg of total protein) in pancreas splenic lobe biopsies. FIG. 29D shows the pancreas biopsy map. FIG. 29E shows group mean VCN in pancreas splenic lobe biopsies collected from vehicle and AAV9.INS.GLP1-treated low and high dose groups. FIG. 29F shows group mean adjusted (Adj.) transgene RNA copies per 10 ng total RNA input in pancreas splenic lobe biopsies collected from vehicle and AAV9.INS.GLP1-treated low and high dose groups. FIG. 29G shows group mean active GLP-1 in pancreas splenic lobe biopsies collected from vehicle and AAV9.INS-GLP1-treated low and high dose groups. FIG. 29H shows lipase measured at the indicated time-points pre- and post-procedure throughout the 6-week study. FIG. 29I shows quantitative immunofluorescence image analysis of all pancreas splenic lobe biopsies presented as group mean values. Percent GLP-1+ beta cells in pancreas splenic lobe islets. FIG. 29J shows percent “functionally transduced islets” (defined as islets containing at least one dual insulin (+)/GLP-1 (+) cell). FIG. 29K shows fold-change (FC) over vehicle in the number of non-beta cells expressing GLP-1 in the pancreas splenic lobe. Data are presented as mean±SEM. n.s., non-significant vs. vehicle (FIGS. 29E-29G: Kruskal-Wallis with Dunn's multiple-comparisons test; FIG. K: one-way ANOVA with Dunnett's multiple-comparisons test).

FIGS. 30A-30I show the targeted, effective, and safe delivery of AAV9.INS.GLP1 via EUS-guided pancreatic infusion in Yucatan pigs. FIG. 30A shows vector copy number (VCN) presented as vector genomes per diploid genome (VG/DG) in pigs administered vehicle. FIG. 30B shows VCN in VG/DG in pigs treated with AAV9.INS.GLP1 at the low dose. FIG. 30C shows VCN in VG/DG in pigs treated with AAV9.INS.GLP1 at the high dose. FIGS. 30D-30F shows adjusted (adj.) transgene RNA copies per 10 ng total RNA input (normalized to mRNA of the PCBP1 housekeeping gene) in pancreatic splenic lobe biopsies. FIGS. 30G-30I show active GLP-1 levels (pg per μg of total protein) in pancreas splenic lobe biopsies.

FIGS. 31A-31D show that no pancreatitis, cardiotoxicity, or hepatoxicity observed following EUS-guided pancreatic delivery of AAV9.INS.GLP1. FIG. 31A shows amylase measured at the indicated time-points pre- and post-procedure throughout the 6-week study for the vehicle group and low and high AAV9.INS.GLP1 dosed groups. Dotted horizontal line indicates the maximum normal threshold. FIG. 31B shows troponin I measured at the indicated time-points pre- and post-procedure throughout the 6-week study. Dotted horizontal line indicates assay Lower Limit of Detection (LLOD). FIG. 31C shows aspartate aminotransferase (AST) measured at the indicated time-points pre- and post-procedure throughout the 6-week. Dotted horizontal lines indicate lower and upper normal thresholds. FIG. 31D shows alanine aminotransferase measured at the indicated time-points pre- and post-procedure throughout the 6-week. Dotted horizontal lines indicate lower and upper normal thresholds.

FIGS. 32A-32F show quantitative immunofluorescence image analysis of all pancreas splenic lobe biopsies presented as group mean values, illustrating beta cell-specific GLP-1 expression in pig pancreas following treatment with AAV9.INS.GLP1. FIG. 32A shows percent GLP-1+ beta cells in the pancreas splenic lobe (SL). FIG. 32B shows fold-change (FC) over vehicle in the number of non-beta cells expressing GLP-1 in the pancreas splenic lobe. FIG. 32C shows percent GLP-1+ beta cells inside the pancreas splenic lobe islets. FIG. 32D shows fold-change (FC) over vehicle in the number of non-beta cells expressing GLP-1 inside the pancreas splenic lobe islets. FIG. 32E shows percent functionally transduced islets (islets containing at least one dual insulin+/GLP-1+ cell) of ≥50 M in area. FIG. 32F shows percent functionally transduced islets of <50 μM in area. Data are presented as mean±SEM. n.s., non-significant vs. vehicle (FIG. 32B: Kruskal-Wallis with Dunn's multiple-comparisons test; FIG. 32D: one-way ANOVA with Dunnett's multiple-comparisons test).

FIGS. 33A-33I show cytokine and complement measurements assessing the innate immune response following AAV9.INS.GLP1 treatment in pigs. FIGS. 33A-33G show a panel of cytokines measured at the indicated time-points throughout the 6-week study in pigs administered vehicle or treated with AAV9.INS.GLP1 at the dose levels indicated in the legend of panel A. FIG. 33H shows early pathway complement response measured via quantitation of C3a. FIG. 33I shows complement response measured via quantitation of terminal complement complex (TCC) C5b-9. Horizontal dotted lines indicate: LLOQ=lower limit of quantitation. ULOQ=upper limit of quantitation. LLOD=lower limit of detection. ULOD—upper limit of detection.

FIGS. 34A-34 show the assessment of humoral and cellular immune response following AAV9.INS.GLP1 treatment in pigs. FIG. 34A shows total antibody (TAb) titers against AAV9 assessed pre-dosing and terminally (6-weeks post-treatment) in vehicle-administered or AAV9.INS.GLP1-treated pigs at both dose levels. Animal identification (ID) indicated. FIG. 34B shows neutralizing antibody (NAb) titers against AAV9 assessed pre-dosing and terminally. FIG. 34C shows anti-drug antibody (ADA) testing on terminal samples against GLP-1 (1-37) peptide (circles) or against a full-length protein consisting of human insulin signal peptide (hINS.sp), GLP-1 (1-37), and intervening peptide 2 (IP2) (squares).

FIGS. 35A-35F show limited off-target biodistribution and transgene RNA expression following EUS-guided AAV9.INS.GLP1 treatment in pigs. FIG. 35A shows pancreas splenic lobe (SL) and off-target biodistribution in the vehicle group measured in the tissue types listed at the bottom. VCN (vector copy number) presented as vector genomes per diploid genome (VG/DG). FIG. 35B shows pancreas splenic lobe (SL) and off-target transgene RNA expression in the vehicle-administered group. Presented as transgene RNA per 10 ng total RNA input adjusted (Adj.) (normalized) to RNA of the PCBP1 housekeeping gene. FIG. 35C shows pancreas splenic lobe (SL) and off-target VCN biodistribution for the low-dose AAV9.INS.GLP1 group. FIG. 35D shows pancreas splenic lobe (SL) and off-target transgene RNA expression for the low-dose AAV9.INS.GLP1 group. FIG. 35E shows off-target VCN biodistribution for the high-dose AAV9.INS.GLP1 group. FIG. 35F shows pancreas splenic lobe (SL) and off-target transgene RNA expression for the high-dose AAV9.INS.GLP1 group.

FIGS. 36A-36D show mean (±SEM) serum concentrations of Amylase (FIG. 36A), Lipase (FIG. 36B), ALT (FIG. 36C), and AST (FIG. 36D) at baseline and across serial timepoints through day 49 following dose-ranged administration of AAV9.INS.GLP1. ULN=2998 U/L in (FIG. 36A), 15 U/L in (FIG. 36B), 58 U/L in (FIG. 36C), and 84 U/L in (FIG. 36D). Lower Limit of Normal (LLN)=17 U/L in (FIG. 36C) and 14 U/L in (FIG. 36D).

FIGS. 37A-37B show troponin I levels (FIG. 37A) and Neurofilament light levels (FIG. 37B) measured over time in animals treated with vehicle (Veh) or AAV9.INS.GLP1 [N=3/group].

FIGS. 38A-38C show fasting plasma C-peptide (FIG. 38A), plasma insulin (FIG. 38B), and serum blood glucose (FIG. 38C) levels measured over time in animals treated with vehicle (Veh) or AAV9.INS.GLP1 [N=3/group]. Data are presented as mean±SEM. Shaded regions in FIG. 38B-38C indicate the reference ranges (LLN, lower limit of normal; ULN, upper limit of normal).

FIG. 39 shows assessment of immunogenicity at pre-dose and terminal timepoints.

FIGS. 40A-40H show transgene biodistribution and expression in the splenic lobe (SL) of the pancreas. FIG. 40A shows vector copy number by biopsy location. FIG. 40B shows transgene RNA copies by biopsy location. FIG. 40C shows active GLP-1 protein by biopsy location. FIG. 40D shows the pancreas biopsy map. FIG. 40E shows average vector copy number in SL by group. FIG. 40F shows average transgene RNA copy number in SL by group.

FIG. 40G shows average active GLP-1 protein in SL by group. FIG. 40H shows dual-positive insulin (INS) GLP-1 co-staining score based on dual insulin GLP-1 immunofluorescence within the pancreas splenic lobe: 0=absent, 1=<10%, 2=11-25%, 3=26-50%, 4=51-75%, 5=>75% of endocrine cells that are co-positive.

FIG. 41 shows functionally transduced islet counts presented as a percentage of transduced islets based on pathologist scoring of dual positives.

FIGS. 42A-42E show VCN levels in pancreas splenic lobe (SL) and in OFT tissues from vehicle-treated animals (FIG. 42A) or AAV9.INS.GLP1 treated animals at low (FIG. 42B), mid (FIG. 42C), and high (FIG. 42D) dose levels. FIG. 42E shows transgene RNA levels in pancreas SL and in all OFT tissue samples from high-dose AAV9.INS.GLP1-treated animals that were found to be positive for viral DNA.

FIGS. 43A-43G show viral genome shedding and clearance measured via ddPCR in feces (FIG. 43A), saliva (FIG. 43B), urine (FIG. 43C), and blood (FIG. 43D) collected across multiple timepoints. (N=3/group, presented as mean±SEM). FIGS. 43E-43G show the clearance trajectory of viral DNA in blood for low, mid, and high-dose individual animals.

FIG. 44 is a flowchart illustrating a method of controlling an infusion procedure, according to some embodiments.

FIG. 45 shows an example of a motorized fluid stop mechanism, according to some embodiments.

FIG. 46 shows an example needle force sensing procedure.

FIG. 47 shows an example portion of a dual-line workflow assembly, according to some embodiments.

FIG. 48 shows an example distal needle having an active beacon that is configured to generate a unique signature, according to some embodiments.

FIG. 49 shows an example distal needle having a magnet disposed thereon, according to some embodiments.

FIG. 50 shows a diagram of a system that illustrates communication among elements of the system, according to some embodiments.

### DETAILED DESCRIPTION

The data provided herein demonstrates that the pancreatic gene therapy compositions of the disclosure can be used not only to reduce weight in obese individuals but also to prevent weight gain (e.g., maintain weight) in non-obese individuals; for example, when faced with dietary changes, such as a high caloric diet (e.g., a high fat diet and/or a high sugar diet), and/or physical changes, such reduced physical activity. These findings were achieved using a gene therapy composition that delivers nutrient-responsive pancreatic-specific production of a GLP-1 receptor agonist. GLP-1 receptor agonists include molecules that can bind to and activate the GLP-1 receptor (GLP-1R). In some embodiments, a GLP-1 receptor agonist includes a GLP-1 peptide (e.g., GLP-1₍₇₋₃₇₎ or GLP-1₍₇₋₃₆₎). In other embodiments, a GLP-1 receptor agonist includes an exendin-4 peptide.

### Pancreatic Gene Therapy Compositions

A “pancreatic gene therapy composition” includes compositions that comprise a polynucleotide encoding a pancreatic islet cell specific promoter operably linked to a transgene encoding a GLP-1 receptor agonist. In some embodiments, a pancreatic gene therapy composition comprises a viral vector (e.g., an adeno-associated virus (AAV) vector) that includes (i) a recombinant AAV genome encoding a pancreatic islet cell specific promoter operably linked to a transgene encoding a GLP-1 receptor agonist and (ii) capsid proteins. It should be understood that a transgene encoding a GLP-1 receptor agonist can also encode additional peptides, such as one or more signal peptide and/or one or more other preproglucagon peptides, discussed elsewhere herein.

Within cells, the preproglucagon (GCG) gene is processed to ultimately liberate GLP-1₍₁₋₃₇₎, which is further cleaved by the PC1/3 enzyme to liberate GLP-1₍₇₋₃₇₎, which is the active form of GLP-1. While simply expressing the active form of GLP-1₍₇₋₃₇₎ in cells might be considered the most straightforward way to deliver a therapeutically effective amount, studies have shown, that approach instead yields low levels of GLP-1 peptide with low activity. Quite unexpectedly, it has been shown that certain elements of the GCG gene encoded by the engineered nucleic acids (e.g., transgene expression cassettes) in some embodiments of the disclosure significantly boost GLP-1 activity to levels needed to achieve a therapeutic benefit. For example, inclusion of an IP2 coding sequence in an engineered nucleic acid encoding a GLP-1 peptide yields higher GLP-1 activity, relative to those engineered nucleic acids that exclude the IP2 coding sequence. This was surprising in part because (i) there is no known function ascribed to the IP2 peptide, (ii) IP2 is the most divergent region within the GCG gene, and (iii) during natural protein processing of GCG in a cell, IP2 is cleaved off, leaving only GLP-1₍₁₋₃₇₎ for further processing. Moreover, none of the GLP-1-based drugs on the market includes an IP2 peptide. Thus, in some embodiments, a pancreatic gene therapy composition includes an engineered nucleic acid that comprises a pancreatic islet cell promoter operably linked to an open reading frame encoding a GLP-1 receptor agonist and an IP2 peptide.

The pancreatic gene therapy compositions described herein, in some aspects, include a viral vector comprising (i) a recombinant viral genome comprising a pancreatic islet cell specific promoter operably linked to a transgene comprising a GLP-1 receptor agonist coding sequence and (ii) viral capsid proteins. In some embodiments, a pancreatic gene therapy composition includes an adeno-associated virus (AAV) vector comprising (i) a recombinant AAV genome comprising a pancreatic islet cell specific promoter operably linked to a transgene comprising a GLP-1 receptor agonist coding sequence and (ii) AAV capsid proteins.

In some embodiments, an AAV genome is from a naturally derived serotype. In some embodiments, a serotype is selected from the group of AAV1, AAV2, AAV4, AAV5, AAV6, AAV8, or AAV9. In some embodiments, the AAV serotype is AAV9. See, e.g., U.S. Pat. No. 7,198,951. In some embodiments, the AAV serotype is AAV2. See, e.g., U.S. Pat. No. 6,962,815.

A recombinant viral genome (e.g., recombinant AAV genome) can be single-stranded or self-complementary. In some embodiments, a recombinant AAV genome is a single-stranded AAV (ssAAV) genome. A ssAAV genome includes a single-stranded DNA with an inverted terminal repeats (ITR) at each end. In some embodiments, a recombinant AAV genome is a self-complementary AAV (scAAV) genome. A scAAV genome includes a modified form of a scAAV that contains a self-complementary, partially double-stranded DNA genome. Such scAAV genomes can be designed by deleting the terminal resolution site (trs) in one of the ITRs, preventing the AAV virus from packaging a full-length single-stranded genome.

A recombinant viral genome (e.g., recombinant AAV genome) includes one or more inverted terminal repeat (ITR) sequences. AAV ITRs, for example, are typically palindromic, self-complementary sequences that form a T-shaped hairpin structure, used for AAV genome replication by facilitating binding and cleavage by Rep proteins at the terminal resolution site. In some embodiments, ITRs flank a transgene in a recombinant AAV genome to enable AAV genome packaging into AAV capsids, thereby producing an AAV vector. In some embodiments, a recombinant AAV genome comprises AAV ITRs. In In some embodiments, a recombinant AAV genome comprises AAV2 ITRs. In some embodiments, an AAV2 ITR comprises the sequence of SEQ ID NO: 11. In some embodiments, an AAV2 ITR comprises the sequence of SEQ ID NO: 12. In some embodiments, a transgene of a recombinant AAV genome is flanked by an AAV2 ITR comprising the sequence of SEQ ID NO: 11 (upstream from/5′ to the transgene) and an AAV2 ITR comprising the sequence of SEQ ID NO: 12 (downstream from/3′ to the transgene).

Recombinant viral genomes (e.g., recombinant AAV genomes) of the disclosure, in some aspects, include a pancreatic islet cell specific promoter operably linked to a transgene comprising a GLP-1 receptor agonist coding sequence.

Pancreatic islet cell specific promoters include regulatory DNA sequences that drive gene expression specifically in pancreatic islet cells, including β cells (insulin-producing), a cells (glucagon-producing), 8-cells (somatostatin-producing), PP-cells (pancreatic polypeptide-producing), and ¿-cells (ghrelin-producing). In some embodiments, a pancreatic islet cell specific promoter is a pancreatic β cell specific promoter. In some embodiments, a pancreatic islet cell specific promoter comprises a human insulin promoter sequence (e.g., one or more copies of a human insulin promoter sequence). In some embodiments, a human insulin promoter sequence comprises the sequence of SEQ ID NO: 6. In some embodiments, a human insulin promoter sequence comprises a sequence having at least 90%, at least 95%, or at least 98% identity to the sequence of SEQ ID NO: 6.

The term “operably linked” is used herein to describe a connection between a (one or more) regulatory element, such as a promoter, and a gene (e.g., transgene) or its coding region. In some embodiments, GLP-1 receptor agonist expression is placed under transcriptional control of a pancreatic islet cell specific promoter, thus is operably linked to the promoter—the promoter controls transcription of the GLP-1 receptor agonist. As is known in the art, a “promoter” includes a nucleotide sequence that permits binding of an RNA polymerase and directs the transcription of a gene, such as a transgene encoding a GLP-1 receptor agonist.

A “transgene” of a recombinant viral genome (e.g., recombinant AAV genome) is a gene that does not occur naturally in the viral genome. In the context of a recombinant AAV genome, a transgene is operably linked to a pancreatic islet cell specific promoter such that the promoter drives expression of the transgene. In some embodiments, a transgene encodes a GLP-1 receptor agonist, such as a human GLP-1₍₇₋₃₇₎ peptide. In some embodiments, a transgene encodes GLP-1₍₁₋₃₇₎. In some embodiments, a transgene encodes GLP-1₍₁₋₃₆₎. In some embodiments, a transgene encodes GLP-1₍₇₋₃₆₎. In some embodiments, a transgene comprises a human insulin signal peptide coding sequence. In some embodiments, a human insulin signal peptide coding sequence comprises the sequence of SEQ ID NO: 13. In some embodiments, a human insulin signal peptide coding sequence comprises a sequence having at least 90%, at least 95%, or at least 98% identity to the sequence of SEQ ID NO: 13. In some embodiments, a transgene comprises a GLP-1₍₁₋₃₇₎ coding sequence. In some embodiments, a GLP-1₍₁₋₃₇₎ coding sequence comprises the sequence of SEQ ID NO: 7. In some embodiments, a GLP-1₍₁₋₃₇₎ coding sequence comprises a sequence having at least 90%, at least 95%, or at least 98% identity to the sequence of SEQ ID NO: 7. In some embodiments, a transgene comprises an IP2 peptide coding sequence. In some embodiments, an IP2 peptide coding sequence comprises the sequence of SEQ ID NO: 8. In some embodiments, an IP2 peptide coding sequence comprises a sequence having at least 90%, at least 95%, or at least 98% identity to the sequence of SEQ ID NO: 8. In some embodiments, a transgene comprises a human insulin signal peptide coding sequence, a GLP-1₍₁₋₃₇₎ coding sequence, and an IP2 peptide coding sequence. In some embodiments, a human insulin signal peptide coding sequence is upstream from a GLP-1₍₁₋₃₇₎ coding sequence, which is upstream from an IP2 peptide coding sequence, for example, as represented in the sequence of SEQ ID NO: 1. In some embodiments, a transgene comprises the sequence of SEQ ID NO: 1. In some embodiments, a transgene comprises a sequence having at least 90% identity to the sequence of SEQ ID NO: 1. In some embodiments, a transgene comprises a sequence having at least 95% identity to the sequence of SEQ ID NO: 1. In some embodiments, a transgene comprises a sequence having at least 98% identity to the sequence of SEQ ID NO: 1.

A recombinant viral genome (e.g., recombinant AAV genome), in some embodiments, includes at least one additional element, for example, that enables efficient transgene delivery and/or expression in target cells. Such additional elements include, without limitation, enhancers, polyadenylation (polyA) signal sequences, a Woodchuck Hepatitis Virus Posttranscriptional Regulatory Element (WPRE), introns, and exons.

In some embodiments, a recombinant viral genome (e.g., recombinant AAV genome) includes a WPRE. This element is known to enhance mRNA stability and translation efficiency. In some embodiments, a recombinant AAV genome includes a WPRE that comprises a sequence of SEQ ID NO: 9. In some embodiments, a recombinant AAV genome includes a WPRE that comprises a sequence having at least 90%, at least 95%, or at least 98% identity to the sequence of SEQ ID NO: 9. In some embodiments, a WPRE is downstream from a GLP-1 receptor agonist coding sequence and/or an IP2 peptide coding sequence.

In some embodiments, a recombinant viral genome (e.g., recombinant AAV genome) includes a polyA signal sequence. A polyA signal sequence is a DNA sequence that directs the addition of a poly(A) tail to the 3′ end of mRNA during transcription. Non-limiting examples of polyA signal sequences that can be used in the recombinant AAV genomes of the disclosure include SV40 polyA (Simian Virus 40) signal sequences, bovine growth hormone (BGH) polyA signal sequences, and human growth hormone (hGH) polyA signal sequences. In some embodiments, a recombinant AAV genome includes a BGH polyA signal sequence. In some embodiments, a recombinant AAV genome includes a BGH polyA signal sequence that comprises the sequence of SEQ ID NO: 10. In some embodiments, a polyA signal sequence is downstream from a WPRE.

In some embodiments, a recombinant viral genome (e.g., recombinant AAV genome) comprises the sequence of SEQ ID NO: 2. In some embodiments, a recombinant AAV genome comprises a sequence having at least 90% identity to the sequence of SEQ ID NO: 2. In some embodiments, a recombinant AAV genome comprises a sequence having at least 95% identity to the sequence of SEQ ID NO: 2. In some embodiments, a recombinant AAV genome comprises a sequence having at least 98% identity to the sequence of SEQ ID NO: 2. In some embodiments, a recombinant AAV genome comprises the sequence of SEQ ID NO: 3. In some embodiments, a recombinant AAV genome comprises a sequence having at least 90% identity to the sequence of SEQ ID NO: 3. In some embodiments, a recombinant AAV genome comprises a sequence having at least 95% identity to the sequence of SEQ ID NO: 3. In some embodiments, a recombinant AAV genome comprises a sequence having at least 98% identity to the sequence of SEQ ID NO: 3.

Some aspects of the technology relate to a recombinant AAV genome comprising in the following 5′ to 3′ order: a first AAV inverted terminal repeat (ITR) sequence, the pancreatic islet cell specific promoter, the transgene, a WPRE, a polyadenylation (polyA) sequence, and a second AAV ITR. In some embodiments, the first AAV inverted terminal repeat (ITR) sequence comprises the sequence of SEQ ID NO: 11, the pancreatic islet cell specific promoter comprises the sequence of SEQ ID NO: 6, the GLP-1 receptor agonist coding sequence of the transgene comprises the sequence of SEQ ID NO: 7, the WPRE comprising the sequence of SEQ ID NO: 9, the polyadenylation (polyA) sequence comprising the sequence of SEQ ID NO: 10, and the second AAV ITR comprising the sequence of SEQ ID NO: 12.

AAV vectors of the disclosure include a recombinant AAV genome and AAV capsid proteins. In some embodiments, the AAV capsid proteins are AAV9 capsid proteins (from an AAV9 serotype). In some embodiments, the AAV capsid proteins are AAV2 capsid proteins (from an AAV2 serotype). AAV capsid proteins include three viral proteins (VP1, VP2, and VP3) that self-assemble into a 60-subunit icosahedral structure. These proteins are encoded by the Cap gene, which is part of the naturally occurring AAV genome.

Some aspects relate to an AAV viral vector comprising a polynucleotide of the disclosure and AAV (e.g., AAV9) capsid proteins. In some embodiments, an AAV viral vector comprises a polynucleotide comprising the nucleotide sequence of SEQ ID NO: 1. In some embodiments, an AAV viral vector comprises a polynucleotide comprising a sequence having at least 90%, at least 95%, or at least 98% identity to the nucleotide sequence of SEQ ID NO: 1. In some embodiments, an AAV viral vector comprises a polynucleotide comprising the nucleotide sequence of SEQ ID NO: 2. In some embodiments, an AAV viral vector comprises a polynucleotide comprising a sequence having at least 90%, at least 95%, or at least 98% identity to the nucleotide sequence of SEQ ID NO: 2. In some embodiments, an AAV viral vector comprises a polynucleotide comprising the nucleotide sequence of SEQ ID NO: 3. In some embodiments, an AAV viral vector comprises a polynucleotide comprising a sequence having at least 90%, at least 95%, or at least 98% identity to the nucleotide sequence of SEQ ID NO: 3.

In some embodiments, an AAV viral vector comprising a recombinant AAV genome comprising the sequence of SEQ ID NO: 3 and AAV (e.g., AAV9) capsid proteins. In some embodiments, an AAV viral vector comprising a recombinant AAV genome comprising a sequence having at least 90% identity to the sequence of SEQ ID NO: 3 and AAV (e.g., AAV9) capsid proteins. In some embodiments, an AAV viral vector comprising a recombinant AAV genome comprising a sequence having at least 95% identity to the sequence of SEQ ID NO: 3 and AAV (e.g., AAV9) capsid proteins. In some embodiments, an AAV viral vector comprising a recombinant AAV genome comprising a sequence having at least 98% identity to the sequence of SEQ ID NO: 3 and AAV (e.g., AAV9) capsid proteins.

Methods of producing AAV vectors are known and may be used to produce pancreatic gene therapy compositions of the disclosure. One such exemplary method is the “triple transfection method” (e.g., as described in U.S. Pat. No. 6,001,650), which uses three different plasmids: one containing a gene of interest (transfer plasmid), another carrying the AAV Rep and Cap genes (Rep/Cap plasmid), and a third providing adenovirus helper genes necessary for AAV replication (helper plasmid). The process generally includes preparing and mixing the plasmids, forming a complex with the transfection reagent, delivering the complex into host cells (e.g., HEK293 cells or Sf9 cells), and allowing gene expression. Other production methods can be used.

Other viral vectors are contemplated herein, for example, lentiviral vectors, retroviral vectors, adenoviral vectors, and herpes simplex virus vectors. In some embodiments, a viral vector is a lentiviral vector. In some embodiments, a viral vector is a retroviral vector. In some embodiments, a viral vector is an adenoviral vector. In some embodiments, a viral vector is a herpes simplex virus vector.

Some aspects of the disclosure provide a recombinant adeno-associated virus (rAAV) vector, comprising: (a) an rAAV vector genome sequence that is at least 80% identical to the nucleotide sequence of SEQ ID NO: 3; and (b) AAV capsid proteins.

In some embodiments, the rAAV vector of claim 1, wherein the rAAV vector genome sequence comprises an open reading frame encoding glucagon-like peptide-1 (GLP-1), optionally GLP-1 (1-37).

In some embodiments, the open reading frame further encodes intervening peptide 2 (IP2).

In some embodiments, the open reading frame further encodes a human insulin signal peptide.

In some embodiments, the open reading frame is operably linked to a pancreatic beta cell-specific promoter.

In some embodiments, the pancreatic beta cell-specific promoter comprises three tandem repeats of a human insulin core promoter.

In some embodiments, the rAAV genome sequence is at least 90% identical to the nucleotide sequence of SEQ ID NO: 3. In some embodiments, the rAAV genome sequence is at least 95% identical to the nucleotide sequence of SEQ ID NO: 3. In some embodiments, the rAAV genome sequence comprises the nucleotide sequence of SEQ ID NO: 3.

In some embodiments, the rAAV vector genome sequence comprises AAV2 inverted terminal repeats (ITRs).

In some embodiments, the AAV capsid proteins comprise AAV9 capsid proteins.

Other aspects of the disclosure provide a gene therapy composition: comprising a rAAV vector of any one of the aspects or embodiments described herein and at least one excipient.

In some embodiments, at least one excipient of the composition is selected from phosphate buffered saline and Poloxamer 188, optionally 0.001% Poloxamer 188.

In some embodiments, the composition has a pH value of about 6.5 to about 7.5, optionally about 7.2.

### Polynucleotides

The disclosure also provides polynucleotides that can be used to produce a GLP-1 receptor agonist, for example, in a nutrient responsive manner. In some embodiments, such polynucleotides are delivered directly to pancreatic tissue in a subject. Some aspects provide a polynucleotide comprising a pancreatic islet cell specific promoter operably linked to a transgene comprising a GLP-1 receptor agonist coding sequence.

In some embodiments, a polynucleotide includes one or more inverted terminal repeat (ITR) sequences. In some embodiments, a polynucleotide comprises AAV ITRs. In some embodiments, a polynucleotide comprises AAV2 ITRs. In some embodiments, an AAV2 ITR comprises the sequence of SEQ ID NO: 11. In some embodiments, an AAV2 ITR comprises the sequence of SEQ ID NO: 11. In some embodiments, a transgene of a polynucleotide is flanked by an AAV2 ITR comprising the sequence of SEQ ID NO: 11 (upstream from/5′ to the transgene) and an AAV2 ITR comprising the sequence of SEQ ID NO: 12 (downstream from/3′ to the transgene).

Polynucleotides of the disclosure, in some aspects, include a pancreatic islet cell specific promoter operably linked to a transgene comprising a GLP-1 receptor agonist coding sequence. In some embodiments, a pancreatic islet cell specific promoter is a pancreatic β cell specific promoter. In some embodiments, a pancreatic islet cell specific promoter comprises a human insulin promoter sequence. In some embodiments, a human insulin promoter sequence comprises the sequence of SEQ ID NO: 6. In some embodiments,

A “transgene” of a polynucleotide is a gene that does not occur naturally in the polynucleotide. In the context of a polynucleotide, a transgene is operably linked to a pancreatic islet cell specific promoter such that the promoter drives expression of the transgene. In some embodiments, a transgene encodes a GLP-1 receptor agonist, such as a human GLP-1 (7-37) peptide. In some embodiments, a transgene encodes GLP-1₍₁₋₃₇₎. In some embodiments, a transgene comprises a human insulin signal peptide coding sequence. In some embodiments, a human insulin signal peptide coding sequence comprises the sequence of SEQ ID NO: 13. In some embodiments, a transgene comprises a GLP-1₍₁₋₃₇₎ coding sequence. In some embodiments, a GLP-1₍₁₋₃₇₎ coding sequence comprises the sequence of SEQ ID NO: 7. In some embodiments, a transgene comprises an IP2 peptide coding sequence. In some embodiments, an IP2 peptide coding sequence comprises the sequence of SEQ ID NO: 8. In some embodiments, a transgene comprises a human insulin signal peptide coding sequence, a GLP-1₍₁₋₃₇₎ coding sequence, and an IP2 peptide coding sequence. In some embodiments, a human insulin signal peptide coding sequence is upstream from a GLP-1₍₁₋₃₇₎ coding sequence, which is upstream from an IP2 peptide coding sequence, for example, as represented in the sequence of SEQ ID NO: 1. In some embodiments, a transgene comprises the sequence of SEQ ID NO: 1. In some embodiments, a transgene comprises a sequence having at least 90% identity to the sequence of SEQ ID NO: 1. In some embodiments, a transgene comprises a sequence having at least 95% identity to the sequence of SEQ ID NO: 1. In some embodiments, a transgene comprises a sequence having at least 98% identity to the sequence of SEQ ID NO: 1.

A polynucleotide, in some embodiments, includes at least one additional element, for example, that enables efficient transgene delivery via a viral vector system, for example, and/or expression in target cells. Such additional elements include, without limitation, enhancers, polyadenylation (polyA) signal sequences, a Woodchuck Hepatitis Virus Posttranscriptional Regulatory Element (WPRE), introns, and exons.

In some embodiments, a polynucleotide includes a WPRE. In some embodiments, a recombinant AAV genome includes a WPRE that comprises a sequence of SEQ ID NO: 9. In some embodiments, a WPRE is downstream from a GLP-1 receptor agonist coding sequence and/or an IP2 peptide coding sequence.

In some embodiments, a polynucleotide includes a polyA signal sequence. In some embodiments, a recombinant AAV genome includes a BGH polyA signal sequence. In some embodiments, a recombinant AAV genome includes a BGH polyA signal sequence that comprises the sequence of SEQ ID NO: 10. In some embodiments, a polyA signal sequence is downstream from a WPRE.

In some embodiments, a polynucleotide comprises the sequence of SEQ ID NO: 2. In some embodiments, a polynucleotide comprises a sequence having at least 90% identity to the sequence of SEQ ID NO: 2. In some embodiments, a polynucleotide comprises a sequence having at least 95% identity to the sequence of SEQ ID NO: 2. In some embodiments, a polynucleotide comprises a sequence having at least 98% identity to the sequence of SEQ ID NO: 2. In some embodiments, a polynucleotide comprises the sequence of SEQ ID NO: 3. In some embodiments, a polynucleotide comprises a sequence having at least 90% identity to the sequence of SEQ ID NO: 3. In some embodiments, a polynucleotide comprises a sequence having at least 95% identity to the sequence of SEQ ID NO: 3. In some embodiments, a polynucleotide comprises a sequence having at least 98% identity to the sequence of SEQ ID NO: 3.

Some aspects of the technology relate to a polynucleotide comprising in the following 5′ to 3′ order: a first AAV inverted terminal repeat (ITR) sequence, the pancreatic islet cell specific promoter, the transgene, a WPRE, a polyadenylation (polyA) sequence, and a second AAV ITR. In some embodiments, the first AAV inverted terminal repeat (ITR) sequence comprises the sequence of SEQ ID NO: 11, the pancreatic islet cell specific promoter comprises the sequence of SEQ ID NO: 6, the GLP-1 receptor agonist coding sequence of the transgene comprises the sequence of SEQ ID NO: 7, the WPRE comprising the sequence of SEQ ID NO: 9, the polyadenylation (polyA) sequence comprising the sequence of SEQ ID NO: 10, and the second AAV ITR comprising the sequence of SEQ ID NO: 12.

Other aspects of the technology relate to a polynucleotides comprising a polynucleotide of the disclosure. In some embodiments, a polynucleotide comprises a polynucleotide comprising the nucleotide sequence of SEQ ID NO: 1. In some embodiments, a polynucleotide comprises a polynucleotide comprising the nucleotide sequence of SEQ ID NO: 2. In some embodiments, a polynucleotide comprises a polynucleotide comprising the nucleotide sequence of SEQ ID NO: 3. In some embodiments, a polynucleotide comprises a polynucleotide comprising the nucleotide sequence of SEQ ID NO: 4.

In some embodiments, a polynucleotide comprises the sequence of SEQ ID NO: 1. In some embodiments, a polynucleotide comprises a sequence having at least 95% identity to the sequence of SEQ ID NO: 1. In some embodiments, a polynucleotide comprises a sequence having at least 90% identity to the sequence of SEQ ID NO: 1. In some embodiments, a polynucleotide comprises the sequence of SEQ ID NO: 2. In some embodiments, a polynucleotide comprises a sequence having at least 95% identity to the sequence of SEQ ID NO: 2. In some embodiments, a polynucleotide comprises a sequence having at least 90% identity to the sequence of SEQ ID NO: 2. In some embodiments, a polynucleotide comprises the sequence of SEQ ID NO: 3. In some embodiments, a polynucleotide comprises a sequence having at least 95% identity to the sequence of SEQ ID NO: 3. In some embodiments, a polynucleotide comprises a sequence having at least 90% identity to the sequence of SEQ ID NO: 3. In some embodiments, a polynucleotide comprises the sequence of SEQ ID NO: 4. In some embodiments, a polynucleotide comprises a sequence having at least 95% identity to the sequence of SEQ ID NO: 4. In some embodiments, a polynucleotide comprises a sequence having at least 90% identity to the sequence of SEQ ID NO: 4.

In some embodiments, a polynucleotide comprises, in the 5′ to 3′ direction: a first AAV inverted terminal repeat (ITR) sequence, a human insulin promoter, a human insulin signal peptide coding sequence, a GLP-1 coding sequence, an IP2 coding sequence, a WPRE sequence, a polyA signal sequence, and a second AAV ITR sequence.

Some aspects of the technology relate to polynucleotides (e.g., DNA) comprising, in the 5′ to 3′ direction: a first AAV2 inverted terminal repeat (ITR) sequence comprising the sequence of SEQ ID NO: 11, a human insulin promoter comprising the sequence of SEQ ID NO: 6, a human insulin signal peptide coding sequence comprising the sequence of SEQ ID NO: 13, a GLP-1 coding sequence comprising the sequence of SEQ ID NO: 7, an IP2 coding sequence comprising the sequence of SEQ ID NO: 8, a WPRE sequence comprising the sequence of SEQ ID NO: 9, a polyA signal sequence comprising the sequence of SEQ ID NO: 10, and a second AAV2 ITR sequence comprising the sequence of SEQ ID NO: 12.

In some embodiments, a polynucleotide comprising the nucleotide sequence of SEQ ID NO: 1. In some embodiments, a polynucleotide comprising the nucleotide sequence of SEQ ID NO: 2. In some embodiments, a polynucleotide comprising the nucleotide sequence of SEQ ID NO: 3.

### Plasmids

The disclosure also provides plasmids that can be used to produce a GLP-1 receptor agonist, for example, in a nutrient responsive manner. A plasmid is a small, circular, double-stranded DNA molecule that is distinct from chromosomal DNA. Some aspects provide a plasmid comprising a pancreatic islet cell specific promoter operably linked to a transgene comprising a GLP-1 receptor agonist coding sequence.

In some embodiments, a plasmid is a transfer plasmid (e.g., one of three vectors used in a triple transfection protocol for the production of AAV vectors). A transfer plasmid includes a transgene (e.g., encoding a GLP-1 receptor agonist) flanked by AAV ITRs, which are necessary for packaging the transgene into the AAV capsid. Components of a plasmid, such as a transfer plasmid, can be selected from ITRs, transgenes, and regulatory elements, such as promoters, polyA signals, enhancers, and other control elements, e.g., to improve gene expression in specific cell types.

In some embodiments, a plasmid includes one or more inverted terminal repeat (ITR) sequences. In some embodiments, a plasmid comprises AAV ITRs. In some embodiments, a plasmid comprises AAV2 ITRs. In some embodiments, an AAV2 ITR comprises the sequence of SEQ ID NO: 11. In some embodiments, an AAV2 ITR comprises the sequence of SEQ ID NO: 11. In some embodiments, a transgene of a plasmid is flanked by an AAV2 ITR comprising the sequence of SEQ ID NO: 11 (upstream from/5′ to the transgene) and an AAV2 ITR comprising the sequence of SEQ ID NO: 12 (downstream from/3′ to the transgene).

Plasmids of the disclosure, in some aspects, include a pancreatic islet cell specific promoter operably linked to a transgene comprising a GLP-1 receptor agonist coding sequence. In some embodiments, a pancreatic islet cell specific promoter is a pancreatic β cell specific promoter. In some embodiments, a pancreatic islet cell specific promoter comprises a human insulin promoter sequence. In some embodiments, a human insulin promoter sequence comprises the sequence of SEQ ID NO: 6. In some embodiments,

A “transgene” of a plasmid is a gene that does not occur naturally in the plasmid. In the context of a plasmid, a transgene is operably linked to a pancreatic islet cell specific promoter such that the promoter drives expression of the transgene. In some embodiments, a transgene encodes a GLP-1 receptor agonist, such as a human GLP-1₍₇₋₃₇₎ peptide. In some embodiments, a transgene encodes GLP-1₍₁₋₃₇₎. In some embodiments, a transgene comprises a human insulin signal peptide coding sequence. In some embodiments, a human insulin signal peptide coding sequence comprises the sequence of SEQ ID NO: 13. In some embodiments, a transgene comprises a GLP-1₍₁₋₃₇₎ coding sequence. In some embodiments, a GLP-1₍₁₋₃₇₎ coding sequence comprises the sequence of SEQ ID NO: 7. In some embodiments, a transgene comprises an IP2 peptide coding sequence. In some embodiments, an IP2 peptide coding sequence comprises the sequence of SEQ ID NO: 8. In some embodiments, a transgene comprises a human insulin signal peptide coding sequence, a GLP-1₍₁₋₃₇₎ coding sequence, and an IP2 peptide coding sequence. In some embodiments, a human insulin signal peptide coding sequence is upstream from a GLP-1₍₁₋₃₇₎ coding sequence, which is upstream from an IP2 peptide coding sequence, for example, as represented in the sequence of SEQ ID NO: 1. In some embodiments, a transgene comprises the sequence of SEQ ID NO: 1. In some embodiments, a transgene comprises a sequence having at least 90% identity to the sequence of SEQ ID NO: 1. In some embodiments, a transgene comprises a sequence having at least 95% identity to the sequence of SEQ ID NO: 1. In some embodiments, a transgene comprises a sequence having at least 98% identity to the sequence of SEQ ID NO: 1.

A plasmid, in some embodiments, includes at least one additional element, for example, that enables efficient transgene delivery via a viral vector system, for example, and/or expression in target cells. Such additional elements include, without limitation, enhancers, polyadenylation (polyA) signal sequences, a Woodchuck Hepatitis Virus Posttranscriptional Regulatory Element (WPRE), introns, and exons.

In some embodiments, a plasmid includes a WPRE. In some embodiments, a recombinant AAV genome includes a WPRE that comprises a sequence of SEQ ID NO: 9. In some embodiments, a WPRE is downstream from a GLP-1 receptor agonist coding sequence and/or an IP2 peptide coding sequence.

In some embodiments, a plasmid includes a polyA signal sequence. In some embodiments, a recombinant AAV genome includes a BGH polyA signal sequence. In some embodiments, a recombinant AAV genome includes a BGH polyA signal sequence that comprises the sequence of SEQ ID NO: 10. In some embodiments, a polyA signal sequence is downstream from a WPRE.

In some embodiments, a plasmid comprises the sequence of SEQ ID NO: 2. In some embodiments, a plasmid comprises a sequence having at least 90% identity to the sequence of SEQ ID NO: 2. In some embodiments, a plasmid comprises a sequence having at least 95% identity to the sequence of SEQ ID NO: 2. In some embodiments, a plasmid comprises a sequence having at least 98% identity to the sequence of SEQ ID NO: 2. In some embodiments, a plasmid comprises the sequence of SEQ ID NO: 3. In some embodiments, a plasmid comprises a sequence having at least 90% identity to the sequence of SEQ ID NO: 3. In some embodiments, a plasmid comprises a sequence having at least 95% identity to the sequence of SEQ ID NO: 3. In some embodiments, a plasmid comprises a sequence having at least 98% identity to the sequence of SEQ ID NO: 3. In some embodiments, a plasmid comprises the sequence of SEQ ID NO: 4. In some embodiments, a plasmid comprises a sequence having at least 90% identity to the sequence of SEQ ID NO: 4. In some embodiments, a plasmid comprises a sequence having at least 95% identity to the sequence of SEQ ID NO: 4. In some embodiments, a plasmid comprises a sequence having at least 98% identity to the sequence of SEQ ID NO: 4.

Some aspects of the technology relate to a plasmid comprising in the following 5′ to 3′ order: a first AAV inverted terminal repeat (ITR) sequence, the pancreatic islet cell specific promoter, the transgene, a WPRE, a polyadenylation (polyA) sequence, and a second AAV ITR. In some embodiments, the first AAV inverted terminal repeat (ITR) sequence comprises the sequence of SEQ ID NO: 11, the pancreatic islet cell specific promoter comprises the sequence of SEQ ID NO: 6, the GLP-1 receptor agonist coding sequence of the transgene comprises the sequence of SEQ ID NO: 7, the WPRE comprising the sequence of SEQ ID NO: 9, the polyadenylation (polyA) sequence comprising the sequence of SEQ ID NO: 10, and the second AAV ITR comprising the sequence of SEQ ID NO: 12.

Other aspects of the technology relate to a plasmids comprising a polynucleotide of the disclosure. In some embodiments, a plasmid comprises a polynucleotide comprising the nucleotide sequence of SEQ ID NO: 1. In some embodiments, a plasmid comprises a polynucleotide comprising the nucleotide sequence of SEQ ID NO: 2. In some embodiments, a plasmid comprises a polynucleotide comprising the nucleotide sequence of SEQ ID NO: 3. In some embodiments, a plasmid comprises a polynucleotide comprising the nucleotide sequence of SEQ ID NO: 4.

In some embodiments, a plasmid comprises the sequence of SEQ ID NO: 1. In some embodiments, a plasmid comprises a sequence having at least 95% identity to the sequence of SEQ ID NO: 1. In some embodiments, a plasmid comprises a sequence having at least 90% identity to the sequence of SEQ ID NO: 1. In some embodiments, a plasmid comprises the sequence of SEQ ID NO: 2. In some embodiments, a plasmid comprises a sequence having at least 95% identity to the sequence of SEQ ID NO: 2. In some embodiments, a plasmid comprises a sequence having at least 90% identity to the sequence of SEQ ID NO: 2. In some embodiments, a plasmid comprises the sequence of SEQ ID NO: 3. In some embodiments, a plasmid comprises a sequence having at least 95% identity to the sequence of SEQ ID NO: 3. In some embodiments, a plasmid comprises a sequence having at least 90% identity to the sequence of SEQ ID NO: 3. In some embodiments, a plasmid comprises the sequence of SEQ ID NO: 4. In some embodiments, a plasmid comprises a sequence having at least 95% identity to the sequence of SEQ ID NO: 4. In some embodiments, a plasmid comprises a sequence having at least 90% identity to the sequence of SEQ ID NO: 4.

In some embodiments, a plasmid comprises, in the 5′ to 3′ direction: a first AAV inverted terminal repeat (ITR) sequence, a human insulin promoter, a human insulin signal peptide coding sequence, a GLP-1 coding sequence, an IP2 coding sequence, a WPRE sequence, a polyA signal sequence, and a second AAV ITR sequence.

### Therapies and Other Uses

The pancreatic gene therapy compositions of the disclosure (e.g., comprising (i) a recombinant AAV genome comprising a pancreatic islet cell specific promoter operably linked to a transgene comprising a GLP-1 receptor agonist coding sequence and (ii) AAV capsid proteins) can be used to treat or prevent a number of metabolic conditions, including obesity, type 2 diabetes, and hyperglycemia, as well as to prevent weight gain (e.g., maintain weight) in the face of dietary and physical lifestyle changes, for example.

### Methods for Preventing Weight Gain

In some embodiments, methods for preventing weight gain in a subject comprise administering to a subject (e.g., injecting pancreatic tissue of the subject) with an effective amount of a pancreatic gene therapy composition comprising a polynucleotide comprising a pancreatic islet cell specific promoter operably linked to a transgene comprising a GLP-1 receptor agonist coding sequence, wherein the subject has a body mass index (BMI) of less than 30 kg/m².

In some embodiments, methods for preventing weight gain in a subject comprise administering to a subject (e.g., injecting pancreatic tissue of the subject) with an effective amount of an adeno-associated virus (AAV) vector comprising (i) a recombinant AAV genome comprising a pancreatic islet cell specific promoter operably linked to a transgene comprising a GLP-1 receptor agonist coding sequence and (ii) AAV (e.g., AAV9) capsid proteins, wherein the subject has a BMI of less than 30 kg/m².

Standard BMI is a person's weight in kilograms divided by the square of height in meters. A high BMI can indicate high body fatness. In general, a person is considered obese if their BMI is 30 or higher. If a subject's BMI is less than 18.5, it falls within the underweight range. If a subject's BMI is 18.5 to <25, it falls within the healthy weight range. If a subject's BMI is 25 to <30, it falls within the overweight range. If a subject's BMI is 30 or higher, it falls within the obesity range. In some embodiments, a subject has a BMI of less than 25 kg/m². In some embodiments, a subject has a BMI of 18.5-24.9 kg/m².

In some embodiments, methods for preventing weight gain can be used for maintaining the weight of a subject, for example, on a high caloric diet (e.g., high fat diet and/or a high sugar diet) for a prolonged period, below a threshold BMI or within a certain percentage of the starting weight of the subject. Thus, in some embodiments, an effective amount of a gene therapy composition for preventing weight gain is sufficient for the subject to maintain a BMI of less than 30 kg/m² on a high caloric diet for at least 7 days. In some embodiments, an effective amount is sufficient for the subject to maintain a BMI of less than 30 kg/m² on a high caloric diet for at least 14 days. In some embodiments, an effective amount is sufficient for the subject to maintain a BMI of less than 30 kg/m² on a high caloric diet for at least 21 days. In some embodiments, an effective amount is sufficient for the subject to maintain a BMI of less than 30 kg/m² on a high caloric diet for at least 28 days. It should be understood that it can take several weeks, for example, for a subject to lose weight following treatment with a pancreatic gene therapy composition of the disclosure. Thus, a subject can be administered a gene therapy composition, and then several (e.g., 2 or more) weeks may maintain his/her starting weight (and/or BMI) before initially losing weight.

A “starting weight” refers to the weight of a subject at the time a pancreatic gene therapy composition is administered (e.g., injected). A “high caloric diet” includes a diet in which the total caloric intake exceeds U.S. Dietary Guidelines for caloric intake, typically exceeding about 2000 kcal/day, for example. High caloric diets can include high fat diets, high sugar diets, and diets high in energy density. In some embodiments, the total caloric intake on a high caloric diet exceeds U.S. Dietary Guidelines (e.g., about 2000 kcal/day) by a least 10%, at least 15%, at least 20%, at least 25%, at least 30%, at least 35%, at least 40%, at least 45%, or at least 50%. In some embodiments, a high caloric diet includes a diet in which the total caloric intake exceeds at least 30% of U.S. Dietary Guidelines for caloric intake. In some embodiments, the total caloric intake on a high caloric diet exceeds U.S. Dietary Guidelines (e.g., about 2000 kcal/day) by a least 500 calories. In some embodiments, the total caloric intake on a high caloric diet exceeds U.S. Dietary Guidelines (e.g., about 2000 kcal/day) by a least 1000 calories. As is known in the art, guidelines for caloric intake often depend on sex, age and/or activity level. For example, U.S. Dietary Recommendations for Caloric Intake for sedentary (little to no physical activity) women is 1,800-2,000 calories per day, for moderately active (engages in moderate physical activity, such as walking or light exercise) women is 2,000-2,200 calories per day, and for active women (engages in regular exercise or has an active lifestyle) is 2,200-2,400 calories per day. U.S. Dietary Recommendations for Caloric Intake for sedentary men is 2,200-2,400 calories per day, for moderately active men is 2,400-2,600 calories per day, and for active men is 2,800-3,000 calories per day.

A “high fat diet” includes a diet in which a significant proportion of total caloric intake comes from fats, typically exceeding 25% (e.g., exceeding 25%, 30%, 35%, or 40%) of daily calories, for example. In some embodiments, a prolonged period is at least 7 days, at least 14 days, at least 21 days, or at least 28 days. In some embodiments, a prolonged period is multiple months (e.g., at least 1, at least, 2, at least 3, at least 4, at least 5, or at least 6 months) or even a year or two years (or more, for example, with multiple doses, e.g., annually or biannually). In some embodiments, a subject undergoing pancreatic gene therapy (e.g., who has received an injection of a pancreatic gene therapy compositions) can maintain a weight that is within 1% of the starting weight of the subject on a high fat diet over a period of at least 7 days. In some embodiments, a subject undergoing pancreatic gene therapy can maintain a weight that is within 2% of the starting weight of the subject on a high fat diet over a period of at least 7 days. In some embodiments, a subject undergoing therapy (i.e., who has received an injection of a pancreatic gene therapy compositions) can maintain a weight that is within 3% of the starting weight of the subject on a high fat diet over a period of at least 7 days. In some embodiments, a subject undergoing therapy (i.e., who has received an injection of a pancreatic gene therapy compositions) can maintain a weight that is within 4% of the starting weight of the subject on a high fat diet over a period of at least 7 days. In some embodiments, a subject undergoing therapy (i.e., who has received an injection of a pancreatic gene therapy compositions) can maintain a weight that is within 5% of the starting weight of the subject on a high fat diet over a period of at least 7 days. In some embodiments, a subject undergoing pancreatic gene therapy (i.e., who has received an injection of a pancreatic gene therapy compositions) can maintain a weight that is within 1% of the starting weight of the subject on a high fat diet over a period of at least 7 days. In some embodiments, a subject undergoing pancreatic gene therapy can maintain a weight that is within 2% of the starting weight of the subject on a high fat diet over a period of at least 7 days. In some embodiments, a subject undergoing therapy can maintain a weight that is within 3% of the starting weight of the subject on a high fat diet over a period of at least 14 days. In some embodiments, a subject undergoing therapy (i.e., who has received an injection of a pancreatic gene therapy compositions) can maintain a weight that is within 4% of the starting weight of the subject on a high fat diet over a period of at least 14 days. In some embodiments, a subject undergoing therapy (i.e., who has received an injection of a pancreatic gene therapy compositions) can maintain a weight that is within 5% of the starting weight of the subject on a high fat diet over a period of at least 14 days. In some embodiments, a subject undergoing therapy can maintain a weight that is within 3% of the starting weight of the subject on a high fat diet over a period of at least 21 days. In some embodiments, a subject undergoing therapy (i.e., who has received an injection of a pancreatic gene therapy compositions) can maintain a weight that is within 4% of the starting weight of the subject on a high fat diet over a period of at least 21 days. In some embodiments, a subject undergoing therapy (i.e., who has received an injection of a pancreatic gene therapy compositions) can maintain a weight that is within 5% of the starting weight of the subject on a high fat diet over a period of at least 21 days. In some embodiments, a subject undergoing therapy can maintain a weight that is within 3% of the starting weight of the subject on a high fat diet over a period of at least 28 days. In some embodiments, a subject undergoing therapy (i.e., who has received an injection of a pancreatic gene therapy compositions) can maintain a weight that is within 4% of the starting weight of the subject on a high fat diet over a period of at least 28 days. In some embodiments, a subject undergoing therapy (i.e., who has received an injection of a pancreatic gene therapy compositions) can maintain a weight that is within 5% of the starting weight of the subject on a high fat diet over a period of at least 28 days.

An effective amount of a pancreatic gene therapy composition for preventing weight gain can vary depending on the patient (i.e., subject), taking into account, for example, sex, age, weight, diet, activity level, lipid profile, blood sugar level, and other lifestyle choices. An effective amount of a composition for preventing weight gain, in some embodiments, is about 1×10¹² to about 5×10¹⁴ AAV vector genomes (VG). For example, an effective amount can be about 1×10¹² AAV VG, 1.5×10¹² AAV VG, 2×10¹² AAV VG, 2.5×10¹² AAV VG, 3×10¹² AAV VG, 3.5×10¹² AAV VG, 4×10¹² AAV VG, 4.5×10¹² AAV VG, 5×10¹² AAV VG, 5.5×10¹² AAV VG, 6×10¹² AAV VG, 6.5×10¹² AAV VG, 7×10¹² AAV VG, 7.5×10¹² AAV VG, 8×10¹² AAV VG, 8.5×10¹² AAV VG, 9×10¹² AAV VG, 9.5×10¹² AAV VG, 1×10¹³ AAV VG, 1.5×10¹³ AAV VG, 2×10¹³ AAV VG, 2.5×10¹³ AAV VG, 3×10¹³ AAV VG, 3.5×10¹³ AAV VG, 4×10¹³ AAV VG, 4.5×10¹³ AAV VG, 5×10¹³ AAV VG, 5.5×10¹³ AAV VG, 6×10¹³ AAV VG, 6.5×10¹³ AAV VG, 7×10¹³ AAV VG, 7.5×10¹³ AAV VG, 8×10¹³ AAV VG, 8.5×10¹³ AAV VG, 9×10¹³ AAV VG, 9.5×10¹³ AAV VG, 1×10¹⁴ AAV VG, 1.5×10¹⁴ AAV VG, 2×10¹⁴ AAV VG, 2.5×10¹⁴ AAV VG, 3×10¹⁴ AAV VG, 3.5×10¹⁴ AAV VG, 4×10¹⁴ AAV VG, 4.5×10¹⁴ AAV VG, or 5×10¹⁴ AAV VG. In some embodiments, an effective amount is about 5×10¹² to about 5×10¹⁴. In some embodiments, an effective amount is about 5×10¹² to about 2.5×10¹⁴. In some embodiments, an effective amount is about 5×10¹² to about 1×10¹⁴.

In some embodiments, an effective amount is 3.5×10¹³ AAV VG, 3.6×10¹³ AAV VG, 3.7×10¹³ AAV VG, 3.8×10¹³ AAV VG, 3.95×10¹³ AAV VG, 4.0×10¹³ AAV VG, 4.1×10¹³ AAV VG, 4.2×10¹³ AAV VG, 4.3×10¹³ AAV VG, 4.4×10¹³ AAV VG, or 4.5×10¹³ AAV VG. In some embodiments, an effective amount is 3.9×10¹³ AAV VG to 4.1×10¹³ AAV VG. In some embodiments, an effective amount is 4.0×10¹³ AAV VG.

In some embodiments, an effective amount is 1.0×10¹⁴ AAV VG, 1.1×10¹⁴ AAV VG, 1.2×10¹⁴ AAV VG, 1.3×10¹⁴ AAV VG, 1.5×10¹⁴ AAV VG, or 1.5×10¹⁴ AAV VG. In some embodiments, an effective amount is 1.1×10¹⁴ AAV VG to 1.31×10¹⁴ AAV VG. In some embodiments, an effective amount is 1.2×10¹⁴ AAV VG.

In some embodiments, an effective amount is 3.0×10¹⁴ AAV VG, 3.1×10¹⁴ AAV VG, 3.2×10¹⁴ AAV VG, 3.3×10¹⁴ AAV VG, 3.4×10¹⁴ AAV VG, 3.5×10¹⁴ AAV VG, 3.6×10¹⁴ AAV VG, 3.7×10¹⁴ AAV VG, 3.8×10¹⁴ AAV VG, 3.9×10¹⁴ AAV VG, or 4.0×10¹⁴ AAV VG. In some embodiments, an effective amount is 3.5×10¹⁴ AAV VG to 3.7×10¹⁴ AAV VG. In some embodiments, an effective amount is 3.6×10¹⁴ AAV VG.

### Methods for Treating Obesity

In some embodiments, methods for treating obesity in a subject comprise administering to a subject (e.g., injecting pancreatic tissue of the subject) with an effective amount of a pancreatic gene therapy composition comprising a polynucleotide comprising a pancreatic islet cell specific promoter operably linked to a transgene comprising a GLP-1 receptor agonist coding sequence, wherein the subject has a body mass index (BMI) of at least 30 kg/m².

In some embodiments, methods for treating obesity in a subject comprise administering to a subject (e.g., injecting pancreatic tissue of the subject) with an effective amount of an adeno-associated virus (AAV) vector comprising (i) a recombinant AAV genome comprising a pancreatic islet cell specific promoter operably linked to a transgene comprising a GLP-1 receptor agonist coding sequence and (ii) AAV (e.g., AAV9) capsid proteins, wherein the subject has a BMI of at least 30 kg/m². Thus, in some embodiments, a subject is an obese subject (e.g. has been diagnosed with obesity).

“Treatment” as it relates to obesity includes maintaining a clinically significant reduction in body weight (e.g., at least 5% reduction relative to starting weight). In some embodiments, treatment of an obese subject with a pancreatic gene therapy composition of the disclosure results in a clinically significant reduction in body weight for at least 3 months. In some embodiments, treatment of an obese subject with a pancreatic gene therapy composition of the disclosure results in a clinically significant reduction in body weight for at least 6 months. In some embodiments, treatment of an obese subject with a pancreatic gene therapy composition of the disclosure results in a clinically significant reduction in body weight for at least 9 months. In some embodiments, treatment of an obese subject with a pancreatic gene therapy composition of the disclosure results in a clinically significant reduction in body weight for at least 1 year. In some embodiments, treatment of an obese subject with a pancreatic gene therapy composition of the disclosure results in the subject having a BMI of less than 30 kg/m² for at least 3 months. In some embodiments, treatment of an obese subject with a pancreatic gene therapy composition of the disclosure results in the subject having a BMI of less than 30 kg/m² for at least 6 months. In some embodiments, treatment of an obese subject with a pancreatic gene therapy composition of the disclosure results in the subject having a BMI of less than 30 kg/m² for at least 9 months. In some embodiments, treatment of an obese subject with a pancreatic gene therapy composition of the disclosure results in the subject having a BMI of less than 30 kg/m² for at least 1 year.

An effective amount of a pancreatic gene therapy composition for treating obesity can vary depending on the particular patient (i.e., subject), taking into account, for example, sex, age, weight, diet, activity level, lipid profile, blood sugar level, and other lifestyle choices. An effective amount of a composition for treating obesity, in some embodiments, is about 1×10¹² to about 5×10¹⁴ AAV vector genomes (VG). For example, an effective amount can be about 1×10¹² AAV VG, 1.5×10¹² AAV VG, 2×10¹² AAV VG, 2.5×10¹² AAV VG, 3×10¹² AAV VG, 3.5×10¹² AAV VG, 4×10¹² AAV VG, 4.5×10¹² AAV VG, 5×10¹² AAV VG, 5.5×10¹² AAV VG, 6×10¹² AAV VG, 6.5×10¹² AAV VG, 7×10¹² AAV VG, 7.5×10¹² AAV VG, 8×10¹² AAV VG, 8.5×10¹² AAV VG, 9×10¹² AAV VG, 9.5×10¹² AAV VG, 1×10¹³ AAV VG, 1.5×10¹³ AAV VG, 2×10¹³ AAV VG, 2.5×10¹³ AAV VG, 3×10¹³ AAV VG, 3.5×10¹³ AAV VG, 4×10¹³ AAV VG, 4.5×10¹³ AAV VG, 5×10¹³ AAV VG, 5.5×10¹³ AAV VG, 6×10¹³ AAV VG, 6.5×10¹³ AAV VG, 7×10¹³ AAV VG, 7.5×10¹³ AAV VG, 8×10¹³ AAV VG, 8.5×10¹³ AAV VG, 9×10¹³ AAV VG, 9.5×10¹³ AAV VG, 1×10¹⁴ AAV VG, 1.5×10¹⁴ AAV VG, 2×10¹⁴ AAV VG, 2.5×10¹⁴ AAV VG, 3×10¹⁴ AAV VG, 3.5×10¹⁴ AAV VG, 4×10¹⁴ AAV VG, 4.5×10¹⁴ AAV VG, or 5×10¹⁴ AAV VG. In some embodiments, an effective amount is about 5×10¹² to about 5×10¹⁴. In some embodiments, an effective amount is about 5×10¹² to about 2.5×10¹⁴. In some embodiments, an effective amount is about 5×10¹² to about 1×10¹⁴.

In some embodiments, an effective amount is 3.5×10¹³ AAV VG, 3.6×10¹³ AAV VG, 3.7×10¹³ AAV VG, 3.8×10¹³ AAV VG, 3.95×10¹³ AAV VG, 4.0×10¹³ AAV VG, 4.1×10¹³ AAV VG, 4.2×10¹³ AAV VG, 4.3×10¹³ AAV VG, 4.4×10¹³ AAV VG, or 4.5×10¹³ AAV VG. In some embodiments, an effective amount is 3.9×10¹³ AAV VG to 4.1×10¹³ AAV VG. In some embodiments, an effective amount is 4.0×10¹³ AAV VG.

In some embodiments, an effective amount is 1.0×10¹⁴ AAV VG, 1.1×10¹⁴ AAV VG, 1.2×10¹⁴ AAV VG, 1.3×10¹⁴ AAV VG, 1.5×10¹⁴ AAV VG, or 1.5×10¹⁴ AAV VG. In some embodiments, an effective amount is 1.1×10¹⁴ AAV VG to 1.31×10¹⁴ AAV VG. In some embodiments, an effective amount is 1.2×10¹⁴ AAV VG.

In some embodiments, an effective amount is 3.0×10¹⁴ AAV VG, 3.1×10¹⁴ AAV VG, 3.2×10¹⁴ AAV VG, 3.3×10¹⁴ AAV VG, 3.4×10¹⁴ AAV VG, 3.5×10¹⁴ AAV VG, 3.6×10¹⁴ AAV VG, 3.7×10¹⁴ AAV VG, 3.8×10¹⁴ AAV VG, 3.9×10¹⁴ AAV VG, or 4.0×10¹⁴ AAV VG. In some embodiments, an effective amount is 3.5×10¹⁴ AAV VG to 3.7×10¹⁴ AAV VG. In some embodiments, an effective amount is 3.6×10¹⁴ AAV VG.

### Methods for Treating Diabetes

In some embodiments, methods for treating diabetes in a subject comprise administering to a subject (e.g., injecting pancreatic tissue of the subject) with an effective amount of a pancreatic gene therapy composition comprising a polynucleotide comprising a pancreatic islet cell specific promoter operably linked to a transgene comprising a GLP-1 receptor agonist coding sequence, wherein the subject has type 2 diabetes.

In some embodiments, methods for treating diabetes, such as type 2 diabetes, in a subject comprise administering to a subject (e.g., injecting pancreatic tissue of the subject) with an effective amount of an adeno-associated virus (AAV) vector comprising (i) a recombinant AAV genome comprising a pancreatic islet cell specific promoter operably linked to a transgene comprising a GLP-1 receptor agonist coding sequence and (ii) AAV (e.g., AAV9) capsid proteins, wherein the subject has type 2 diabetes.

Thus, in some embodiments, a diabetic subject is an obese subject (e.g. has been diagnosed with obesity). In some embodiments, a diabetic subject has a BMI of at least 30 kg/m². In some embodiments, a diabetic subject has a BMI of at least 25 kg/m².

Diabetes is a chronic metabolic disorder characterized by high blood sugar (glucose) levels due to insufficient insulin production and/or ineffective insulin use. “Diabetes” includes: type 1 diabetes, which is an autoimmune condition whereby an immune system attacks insulin-producing cells in the pancreas, leading to little or no insulin production; type 2 diabetes, which is a condition whereby the body becomes resistant to insulin or does not produce enough insulin to maintain normal blood sugar levels. In some embodiments, the methods described herein are used to treat type 2 diabetes. In other embodiments, the methods described herein are used to treat type 1 diabetes.

“Treatment” as it relates to type 2 diabetes includes maintaining a clinically significant reduction in fasting blood glucose (e.g., at least 5% reduction relative to starting fasting blood glucose). A “starting fasting blood glucose” refers to the fasting blood glucose of a subject at the time a pancreatic gene therapy composition is administered (e.g., injected). In some embodiments, treatment of a diabetic subject with a pancreatic gene therapy composition of the disclosure results in a clinically significant reduction in fasting blood glucose for at least 3 months. In some embodiments, treatment of a diabetic subject with a pancreatic gene therapy composition of the disclosure results in a clinically significant reduction in fasting blood glucose for at least 6 months. In some embodiments, treatment of a diabetic subject with a pancreatic gene therapy composition of the disclosure results in a clinically significant reduction in fasting blood glucose for at least 9 months. In some embodiments, treatment of a diabetic subject with a pancreatic gene therapy composition of the disclosure results in a clinically significant reduction in fasting blood glucose for at least 1 year. In some embodiments, treatment of type 2 diabetes includes lowering fasting blood glucose in a subject to below 126 mg/dL.

An effective amount of a pancreatic gene therapy composition for treating type 2 diabetes can vary depending on the particular patient (i.e., subject), taking into account, for example, sex, age, weight, diet, activity level, lipid profile, blood sugar level, and other lifestyle choices. In some embodiments, an effective amount is sufficient to lower fasting blood glucose in the subject to below 126 mg/dL. Fasting blood glucose (FBG) refers to the level of glucose in the blood after an individual has not eaten for an extended period of time, for example, at least 8-12 hours (e.g., measured in the morning before breakfast). Normal FBG is 70-99 mg/dl (3.9-5.5 mmol/L), FBG in a prediabetic subject (also referred to as impaired fasting glucose) is 100-125 mg/dL (5.6-6.9 mmol/L), and FBG in a diabetic subject is ≥126 mg/dl (≥7.0 mmol/L) measured, for example, on two separate occasions. In some embodiments, an effective amount is sufficient to lower fasting blood glucose in the subject to below 100 mg/dL.

In some embodiments, an effective amount is sufficient for a subject to maintain a BMI of less than 30 kg/m² for at least 7 days. In some embodiments, an effective amount is sufficient for a subject to maintain a BMI of less than 30 kg/m² for at least 14 days. In some embodiments, an effective amount is sufficient for a subject to maintain a BMI of less than 30 kg/m² for at least 21 days. In some embodiments, an effective amount is sufficient for a subject to maintain a BMI of less than 30 kg/m² for at least 28 days. In some embodiments, an effective amount is sufficient for a subject to maintain a BMI of less than 30 kg/m² for at least 6 months. In some embodiments, an effective amount is sufficient for a subject to maintain a BMI of less than 30 kg/m² for at least 1 year.

An effective amount of a composition for treating diabetes, such as type 2 diabetes, in some embodiments, is about 1×10¹² to about 5×10¹⁴ AAV vector genomes (VG). For example, an effective amount can be about 1×10¹² AAV VG, 1.5×10¹² AAV VG, 2×10¹² AAV VG, 2.5×10¹² AAV VG, 3×10¹² AAV VG, 3.5×10¹² AAV VG, 4×10¹² AAV VG, 4.5×10¹² AAV VG, 5×10¹² AAV VG, 5.5×10¹² AAV VG, 6×10¹² AAV VG, 6.5×10¹² AAV VG, 7×10¹² AAV VG, 7.5×10¹² AAV VG, 8×10¹² AAV VG, 8.5×10¹² AAV VG, 9×10¹² AAV VG, 9.5×10¹² AAV VG, 1×10¹³ AAV VG, 1.5×10¹³ AAV VG, 2×10¹³ AAV VG, 2.5×10¹³ AAV VG, 3×10¹³ AAV VG, 3.5×10¹³ AAV VG, 4×10¹³ AAV VG, 4.5×10¹³ AAV VG, 5×10¹³ AAV VG, 5.5×10¹³ AAV VG, 6×10¹³ AAV VG, 6.5×10¹³ AAV VG, 7×10¹³ AAV VG, 7.5×10¹³ AAV VG, 8×10¹³ AAV VG, 8.5×10¹³ AAV VG, 9×10¹³ AAV VG, 9.5×10¹³ AAV VG, 1×10¹⁴ AAV VG, 1.5×10¹⁴ AAV VG, 2×10¹⁴ AAV VG, 2.5×10¹⁴ AAV VG, 3×10¹⁴ AAV VG, 3.5×10¹⁴ AAV VG, 4×10¹⁴ AAV VG, 4.5×10¹⁴ AAV VG, or 5×10¹⁴ AAV VG. In some embodiments, an effective amount is about 5×10¹² to about 5×10¹⁴. In some embodiments, an effective amount is about 5×10¹² to about 2.5×10¹⁴. In some embodiments, an effective amount is about 5×10¹² to about 1×10¹⁴.

In some embodiments, an effective amount is 3.5×10¹³ AAV VG, 3.6×10¹³ AAV VG, 3.7×10¹³ AAV VG, 3.8×10¹³ AAV VG, 3.95×10¹³ AAV VG, 4.0×10¹³ AAV VG, 4.1×10¹³ AAV VG, 4.2×10¹³ AAV VG, 4.3×10¹³ AAV VG, 4.4×10¹³ AAV VG, or 4.5×10¹³ AAV VG. In some embodiments, an effective amount is 3.9×10¹³ AAV VG to 4.1×10¹³ AAV VG. In some embodiments, an effective amount is 4.0×10¹³ AAV VG.

In some embodiments, an effective amount is 1.0×10¹⁴ AAV VG, 1.1×10¹⁴ AAV VG, 1.2×10¹⁴ AAV VG, 1.3×10¹⁴ AAV VG, 1.5×10¹⁴ AAV VG, or 1.5×10¹⁴ AAV VG. In some embodiments, an effective amount is 1.1×10¹⁴ AAV VG to 1.31×10¹⁴ AAV VG. In some embodiments, an effective amount is 1.2×10¹⁴ AAV VG.

In some embodiments, an effective amount is 3.0×10¹⁴ AAV VG, 3.1×10¹⁴ AAV VG, 3.2×10¹⁴ AAV VG, 3.3×10¹⁴ AAV VG, 3.4×10¹⁴ AAV VG, 3.5×10¹⁴ AAV VG, 3.6×10¹⁴ AAV VG, 3.7×10¹⁴ AAV VG, 3.8×10¹⁴ AAV VG, 3.9×10¹⁴ AAV VG, or 4.0×10¹⁴ AAV VG. In some embodiments, an effective amount is 3.5×10¹⁴ AAV VG to 3.7×10¹⁴ AAV VG. In some embodiments, an effective amount is 3.6×10¹⁴ AAV VG.

### Methods for Regulating Blood Glucose Levels

In some embodiments, methods for regulating blood glucose in a subject comprise administering to a subject (e.g., injecting pancreatic tissue of the subject) with an effective amount of a pancreatic gene therapy composition comprising a polynucleotide comprising a pancreatic islet cell specific promoter operably linked to a transgene comprising a GLP-1 receptor agonist coding sequence, wherein the subject has a fasting blood glucose of at least 100 mg/dL.

In some embodiments, methods for regulating blood glucose in a subject comprise administering to a subject (e.g., injecting pancreatic tissue of the subject) with an effective amount of an adeno-associated virus (AAV) vector comprising (i) a recombinant AAV genome comprising a pancreatic islet cell specific promoter operably linked to a transgene comprising a GLP-1 receptor agonist coding sequence and (ii) AAV (e.g., AAV9) capsid proteins, wherein the subject has a fasting blood glucose of at least 100 mg/dL. In some embodiments, a subject has a fasting blood glucose of at least 126 mg/dL. Thus, in some embodiments, a subject is a prediabetic or diabetic subject. In some embodiments, a subject is diagnosed with hyperglycemia. In some embodiments, a subject is an obese subject. In some embodiments, a diabetic subject has a BMI of at least 25 kg/m². In some embodiments, a diabetic subject has a BMI of at least 30 kg/m².

Hyperglycemia (high blood sugar) occurs when blood glucose levels exceed normal ranges (e.g., fasting blood glucose <100 mg/dL). It is a hallmark of diabetes mellitus (type 1, type 2, gestational) but can also occur due to stress, medications, or infections, for example. Thus, methods for regulating blood glucose described herein can be used to treat or prevent hyperglycemia.

An effective amount of a pancreatic gene therapy composition for regulating blood glucose can vary depending on the particular patient (i.e., subject), taking into account, for example, sex, age, weight, diet, activity level, lipid profile, blood sugar level, and other lifestyle choices. In some embodiments, an effective amount is sufficient to lower fasting blood glucose in the subject to below 126 mg/dL. In some embodiments, an effective amount is sufficient to lower fasting blood glucose in the subject to below 100 mg/dL.

An effective amount of a composition for regulating blood glucose, in some embodiments, is about 1×10¹² to about 5×10¹⁴ AAV vector genomes (VG). For example, an effective amount can be about 1×10¹² AAV VG, 1.5×10¹² AAV VG, 2×10¹² AAV VG, 2.5×10¹² AAV VG, 3×10¹² AAV VG, 3.5×10¹² AAV VG, 4×10¹² AAV VG, 4.5×10¹² AAV VG, 5×10¹² AAV VG, 5.5×10¹² AAV VG, 6×10¹² AAV VG, 6.5×10¹² AAV VG, 7×10¹² AAV VG, 7.5×10¹² AAV VG, 8×10¹² AAV VG, 8.5×10¹² AAV VG, 9×10¹² AAV VG, 9.5×10¹² AAV VG, 1×10¹³ AAV VG, 1.5×10¹³ AAV VG, 2×10¹³ AAV VG, 2.5×10¹³ AAV VG, 3×10¹³ AAV VG, 3.5×10¹³ AAV VG, 4×10¹³ AAV VG, 4.5×10¹³ AAV VG, 5×10¹³ AAV VG, 5.5×10¹³ AAV VG, 6×10¹³ AAV VG, 6.5×10¹³ AAV VG, 7×10¹³ AAV VG, 7.5×10¹³ AAV VG, 8×10¹³ AAV VG, 8.5×10¹³ AAV VG, 9×10¹³ AAV VG, 9.5×10¹³ AAV VG, 1×10¹⁴ AAV VG, 1.5×10¹⁴ AAV VG, 2×10¹⁴ AAV VG, 2.5×10¹⁴ AAV VG, 3×10¹⁴ AAV VG, 3.5×10¹⁴ AAV VG, 4×10¹⁴ AAV VG, 4.5×10¹⁴ AAV VG, or 5×10¹⁴ AAV VG. In some embodiments, an effective amount is about 5×10¹² to about 5×10¹⁴. In some embodiments, an effective amount is about 5×10¹² to about 2.5×10¹⁴. In some embodiments, an effective amount is about 5×10¹² to about 1×10¹⁴.

In some embodiments, an effective amount is 3.5×10¹³ AAV VG, 3.6×10¹³ AAV VG, 3.7×10¹³ AAV VG, 3.8×10¹³ AAV VG, 3.95×10¹³ AAV VG, 4.0×10¹³ AAV VG, 4.1×10¹³ AAV VG, 4.2×10¹³ AAV VG, 4.3×10¹³ AAV VG, 4.4×10¹³ AAV VG, or 4.5×10¹³ AAV VG. In some embodiments, an effective amount is 3.9×10¹³ AAV VG to 4.1×10¹³ AAV VG. In some embodiments, an effective amount is 4.0×10¹³ AAV VG.

In some embodiments, an effective amount is 1.0×10¹⁴ AAV VG, 1.1×10¹⁴ AAV VG, 1.2×10¹⁴ AAV VG, 1.3×10¹⁴ AAV VG, 1.5×10¹⁴ AAV VG, or 1.5×10¹⁴ AAV VG. In some embodiments, an effective amount is 1.1×10¹⁴ AAV VG to 1.31×10¹⁴ AAV VG. In some embodiments, an effective amount is 1.2×10¹⁴ AAV VG.

In some embodiments, an effective amount is 3.0×10¹⁴ AAV VG, 3.1×10¹⁴ AAV VG, 3.2×10¹⁴ AAV VG, 3.3×10¹⁴ AAV VG, 3.4×10¹⁴ AAV VG, 3.5×10¹⁴ AAV VG, 3.6×10¹⁴ AAV VG, 3.7×10¹⁴ AAV VG, 3.8×10¹⁴ AAV VG, 3.9×10¹⁴ AAV VG, or 4.0×10¹⁴ AAV VG. In some embodiments, an effective amount is 3.5×10¹⁴ AAV VG to 3.7×10¹⁴ AAV VG. In some embodiments, an effective amount is 3.6×10¹⁴ AAV VG.

### Exemplary Endoscopic Ultrasound-Guided Fine Needle Injection (EUS-FNI) Methods

Pancreatic gene therapy compositions of the disclosure, in some embodiments, are delivered to pancreatic tissue of a subject via endoscopic ultrasound-guided fine needle injection (EUS-FNI).

In some embodiments, the injecting of the pancreatic tissue comprises a single infusion.

In some embodiments, the injecting of the pancreatic tissue comprises a double infusion. In some embodiments, an infusion system is used which permits multiple (e.g., two) infusions to be made using a single cartridge of a gene therapy composition of the types described herein. The multiple (e.g., two) infusions may be achieved by actuating a drive mechanism of the infusion system. The drive mechanism may include mechanical components configured to perform a partial infusion in response to a first actuation, and a distinct partial infusion in response to a second actuation. In some embodiments, partial infusions may also, or alternatively, be achieved using a fluid stop mechanism of the infusion system. The fluid stop mechanism may include a mechanical component configured to clamp a lumen of the infusion system through which the gene therapy composition flows.

Some aspects herein provide methods of treating Type 2 diabetes in a human subject in need thereof, comprising: administering, by endoscopic ultrasound-guided intraparenchymal infusion (EUS-IPI), a therapeutically effective amount of the gene therapy composition of any one of claims 12-14 to pancreatic tissue of the human subject. In some embodiments, the EUS-IPI is performed using a delivery device comprising: (a) an infusion needle having a gauge of 27 G, a conical tip, and a side opening proximal to the conical tip, wherein the infusion needle comprises an echogenic coating on at least a portion of its outer surface; (b) a sheath surrounding the infusion needle, wherein the sheath is adjustable in length to accommodate an echoendoscope having a working channel diameter of at least 2.8 mm; and (c) a handle attached to the sheath and having an integrated syringe pump, wherein administering the therapeutically effective amount of the gene therapy composition comprises delivering the gene therapy composition through the infusion needle at a controlled flow rate and pressure by actuating the syringe pump. In some embodiments, the infusion is at a controlled pressure and rate to avoid pancreatic injury.

In some embodiments, the therapeutically effective amount is a single dose of about 3.0×10¹³ vector genomes (VG) to about 5.0×10¹³ VG, preferably 4.0×10¹³ VG. In some embodiments, the therapeutically effective amount is a single dose of about 1.0×10¹⁴ VG to about 2.0×10¹⁴ VG, preferably 1.2×10¹⁴ VG.

In some embodiments, the therapeutically effective amount is administered via two injections into the body and/or tail of the pancreas, optionally two 1 mL injections.

### Delivery Systems

Aspects of the present technology provide infusion systems for infusing gene therapy compositions of the types described herein into a subject. Aspects of the present technology provide the infusion systems in combination with the gene therapy compositions. In some embodiments, the gene therapy composition is infused into the pancreas of the subject. The pancreas may include target cells, such as Beta cells, to be targeted with the gene therapy composition. The infusion system may be a handheld system designed to deliver the gene therapy composition to the pancreas via an endoscopic procedure.

### Device Structure and Components

### General Configuration

According to an aspect of the present technology, the infusion system comprises a handheld tool configured to couple to an endoscope. The infusion system may include a handle, a lumen, and a needle. The handle may be configured to hold a cartridge or syringe of the gene therapy composition. The handle may include a drive mechanism configured to drive the gene therapy composition through the lumen to the needle in one or more infusions. The infusion system may be coupled to an endoscope with the lumen of the infusion system passing through the endoscope channel.

Thus, it should be appreciated from the foregoing that embodiments described herein relate to a handheld infusion system configured for minimally invasive fluid delivery, such as in endoscopic ultrasound (EUS)-guided procedures. The handheld infusion system in some embodiments includes a handle body configured to securely receive and hold a medicament container, such as a pre-filled syringe, and an elongated outer sheath assembly configured to extend through an endoscope working channel and into proximity with targeted tissue, such as a pancreas.

### Handle Body

In some examples, the handle is designed for one-handed operation with, for example, a maximum extended length of around 11 inches and a fully collapsed length of about 7 inches, and a representative and non-limiting diameter range from approximately 0.75 to 1.5 inches. These dimensions are subject to adjustment based on ergonomic considerations and specific procedural requirements, underscoring the modular and adaptable nature of the design.

The handle body in some embodiments houses a medicament container receptacle featuring a rotatable or otherwise moveable cover that securely retains the syringe or other medicament container. A drive mechanism within the handle provides controlled, repeatable force application to the medicament container's plunger, using elements such as constant-force springs, thereby driving fluid from the medicament container through the inner fluid lumen and into targeted tissue.

Example systems can operate within a broad range of specifications to accommodate various clinical applications and performance requirements. For example, in a representative embodiment, the fluid pressure within the syringe barrel is expected to peak at approximately 16 psi, which dissipates to nearly 0 psi at the distal needle. This pressure level correlates to a high-end flow rate of about 1 mL/min; however, example devices can have operation at pressures as low as 1 psi and up to 30-50 psi. Achieving higher pressures may necessitate enlarging the handle—either by increasing its length to accommodate additional constant force springs or by increasing its diameter—or even transitioning to motorized drive systems, such as described herein.

Example embodiments also include a reusable battery operated (e.g., rechargeable) handle that a medicament syringe is inserted into. In some examples, the handle can have controls to adjust dose volumes, flow rates, number of doses, and other infusion parameters.

### Infusion Needle

An inner fluid lumen assembly, which is disposed within and longitudinally moveable relative to the outer sheath, includes a flexible inner fluid lumen terminating distally at an infusion needle. The needle may be constructed to facilitate infusion into the pancreas from the stomach. Thus, in some embodiments, the infusion needle may have a conically tapered, pencil-point geometry and a lateral fluid opening, optimized to minimize puncture forces and reduce tissue trauma (e.g., to reduce pancreatitis). In some examples, the needle has a 27-gauge (“27 G”) diameter, providing advantageous reductions in puncture force and improving needle durability over repeated punctures as may be performed to provide multiple infusions across different locations of the pancreas or other target tissue. In some examples, the needle has a diameter less than 27 G. In some embodiments, smaller diameter needles may be used, such as 29 G.

### Inner Fluid Lumen Assembly

The inner fluid lumen assembly in some embodiments features a multi-part construction, with a flexible inner lining, such as nylon, for reduced viral adsorption, surrounded by a stiffer outer shell, such as a laser-cut stainless-steel tube, that imparts sufficient column strength for effective transmission of axial puncture forces from the handle to the distal needle tip. Viral particles of the gene therapy composition may be susceptible to adsorption on the inner walls of the lumen. Selection of a material such as nylon may reduce or eliminate such adsorption, thereby saving a considerable percentage of viral particles and increasing the effectiveness of the treatment. A proximal thrust flange is integrated into the inner fluid lumen assembly, enabling direct transfer of longitudinal forces from the handle body to the infusion needle. Examples include a small fluid dead volume (e.g., the total volume of fluid contained between the distal end needle opening and the medicament container), the inner fluid lumen can be designed with an approximate internal diameter of 0.013 inches. This configuration maintains a dead volume below 0.25 mL, other examples include this value reduced to as low as 0.05 mL or increased. Examples include the use of a laser cut tube with variability in the cut pattern to provide stiffness that is tuned depending on where the section is along the length of the fluid lumen. In some examples, the tube is a thin-wall hypotube. In some examples, the fluid lumen has a laminate construction with a braid, and the pitch of the braid can vary along the length of the lumen.

In further embodiments of the present technology, the system can comprise additional structural and operational refinements enhancing both clinical versatility and procedural precision. For example, the infusion assembly can incorporate a multi-part shaft designed to optimize flexibility, strength, and control during delivery. Specifically, in some embodiments, the infusion assembly shaft can have a three-part construction comprising an outer sheath, within which is disposed an inner shaft that is movable relative to the outer sheath. The inner shaft can further have a two-part structure, comprising an inner flexible fluid lumen, such as one made from nylon or another polymeric material with low friction and high flexibility, surrounded by a stiffer outer tube connected distally to the infusion needle. The stiffer outer tube is configured to deliver the puncture force necessary to penetrate tissue and can be composed of a metal tube, such as a laser-cut steel tube, with sufficient flexibility to navigate tortuous anatomy yet possessing adequate column strength to resist buckling when advancing the needle into targeted tissue.

Additionally, embodiments of the system can include structural enhancements for improved transmission of puncture force from the proximal hand-held infusion device to the distal needle. Specifically, the proximal end of the inner fluid lumen can comprise a radial flange configured to fixedly secure the inner fluid lumen directly to the hand-held infusion pump device. This fixed radial flange ensures effective axial load transfer, allowing the proximal infusion pump device and the inner fluid lumen (with attached distal needle) to move as a unitary structure. Consequently, puncture force can be applied consistently, precisely, and predictably from the proximal end of the device to the distal needle tip, thereby enhancing accuracy of needle placement and reducing the likelihood of buckling or misalignment during tissue puncture and infusion.

### Sliding Interface Friction Reduction Between Inner Fluid Lumen Assembly and Outer Sheath

In further example embodiments of the present technology, the infusion system incorporates design iterations configured to reduce sliding friction between an outer diameter (OD) of the inner fluid lumen assembly and an inner diameter (ID) of the outer sheath assembly. Reducing sliding friction may improve device usability by lowering the required actuation force at the handle during needle deployment and retraction, improving tactile fidelity and controllability, reducing “stick-slip” behavior, and reducing the risk of sudden jumps in needle motion that could contribute to inaccurate targeting or undesired tissue trauma. In addition, reduced friction may enable more consistent force transmission to the distal needle tip (e.g., during puncture) and may reduce internal wear debris generation over repeated cycles of advancement and retraction.

In some embodiments, friction reduction is achieved through the selection and application of surface treatments and/or lubricious coatings on one or both of the mating sliding surfaces, including the OD of the inner fluid lumen assembly and/or the ID of the outer sheath. In another example embodiment, the OD of the inner fluid lumen assembly includes a lubricious coating selected to reduce the coefficient of friction against the sheath ID, such as a hydrophilic lubricious coating, a PTFE-based coating, a dry-film lubricant, a silicone-based coating (e.g., silicone oil, silicone dispersion, or crosslinked silicone), a fluoropolymer coating, a parylene coating, or other low-friction surface treatment. In certain embodiments, the sheath ID includes a coating or surface treatment configured to reduce friction, such as a hydrophilic coating, hydrophobic coating, plasma-treated surface, or deposited low-friction layer. In further embodiments, one or both surfaces are plasma treated (e.g., to alter surface energy or improve coating adhesion), and thereafter coated with a lubricious material to achieve a durable low-friction sliding interface under wet conditions encountered in clinical use.

In another example embodiment, friction reduction is achieved by providing a controlled lubricating medium at the sliding interface. For example, the device may be provided with a pre-applied lubricant on the OD of the inner fluid lumen assembly and/or the ID of the sheath, such as a thin silicone oil layer, a silicone dispersion that dries to a lubricious film, a crosslinked silicone coating configured to resist migration, a fluorinated lubricant, or a dry-film lubricant. In certain embodiments, the lubricant is applied in a manufacturing step and is configured to remain stable under sterilization and storage conditions. In other embodiments, the device is configured such that an aqueous fluid (e.g., saline, priming fluid, or another compatible fluid) introduced during preparation can wet the sliding interface to provide fluid-assisted lubrication during advancement and retraction, thereby reducing actuation force and smoothing motion.

In another example embodiment, friction reduction is achieved through design of the outer tube portion of the inner fluid lumen assembly, such as when the outer tube comprises a laser-cut tube (LCT) providing column strength and flexibility. In some embodiments, the LCT cut pattern is tuned not only for stiffness but also for frictional interaction with the sheath, such that regions requiring higher flexibility may also be configured with reduced radial protrusions or reduced edge exposure to lower contact forces with the sheath ID. In certain embodiments, the LCT is covered by a secondary sleeve or covering layer configured to present a smoother exterior surface, such as a heat-shrink polymer layer disposed over the LCT, a PTFE sleeve over the outer tube, a thin polymer jacket, or a laminated outer layer. In further embodiments, cut edges of the LCT are smoothed, deburred, electropolished, tumbled, chemically polished, or otherwise treated to reduce asperities and micro-edge features that could increase friction, abrade the sheath ID, or generate particulate debris during repeated sliding.

In some embodiments, the frictional characteristics are varied along the length of the inner fluid lumen assembly to optimize overall performance. For example, a proximal region of the inner fluid lumen assembly may include a relatively low-friction outer surface to reduce handle actuation force during long-stroke motion, while a distal region may maintain slightly higher lateral support to stabilize the needle tip during puncture and infusion. In such embodiments, the device may include discrete zones of differing coatings, sleeves, cut patterns, or surface finishes, thereby tuning friction accommodation along the length of travel. In further embodiments, a PTFE sleeve or heat shrink jacket is applied selectively to only those regions where sliding loads are highest, while leaving other regions unjacketed to preserve flexibility and/or radial compactness.

In another example embodiment, friction reduction is achieved by selecting an ID of the outer sheath to provide an appropriate radial clearance between the OD of the inner fluid lumen assembly and the ID of the sheath. In such embodiments, the radial clearance is selected to reduce sliding friction while maintaining sufficient lateral support to prevent buckling of the inner fluid lumen assembly during axial loading. In certain embodiments, the radial clearance is selected within a range of approximately 0.00075 inches to approximately 0.0135 inches, inclusive of intermediate values, depending on material choices, expected curvature through the endoscope channel, and the required column strength for puncture. In some embodiments, the sheath ID is locally enlarged at one or more segments (e.g., a proximal low-curvature segment) to reduce friction while maintaining tighter clearance in other segments (e.g., a distal segment) to preserve lateral support and reduce whip or radial play.

In another example embodiment, the ID of the sheath is textured or patterned to reduce the effective contact area between the sheath and the inner fluid lumen assembly and thereby reduce friction. In some embodiments, the sheath ID includes micro-ridges, ribbed features, dimpled features, low-contact bearing zones, spiral ridges, a helical inner surface ridge, or other patterning configured to reduce surface-to-surface contact. In certain embodiments, the sheath ID has a non-circular profile such as a tri-lobed lumen profile, wherein the inner fluid lumen assembly contacts the sheath at discrete points or lines rather than over a full circumferential area, thereby reducing friction while maintaining guidance and lateral support. In further embodiments, the textured or patterned sheath ID is formed by extrusion tooling, laser machining, molding, or mechanical forming processes, and may be combined with a lubricious coating to further reduce friction and stick-slip.

In some embodiments, the system employs complementary hydrophilic and hydrophobic sliding surfaces to optimize lubrication performance under wet conditions. In one example embodiment, the sheath ID includes a hydrophilic coating that becomes highly lubricious when wetted by priming fluid or bodily fluids, while the OD of the inner fluid lumen assembly includes a hydrophobic or fluorinated coating configured to resist adsorption and reduce drag. In another example embodiment, the sheath ID is hydrophobic and the inner lumen OD is hydrophilic, such that a lubricating fluid film is retained preferentially at the interface during motion. In certain embodiments, the device is configured to intentionally wet the interface (e.g., via a priming step) before needle deployment, thereby enabling fluid-assisted lubrication and smoother advancement through tortuous anatomy.

In further embodiments, friction reduction features are integrated with existing device structures described herein, including the multi-part inner lumen construction, the outer sheath assembly configured to traverse the endoscope working channel, and the handle drive mechanisms configured to deliver puncture force and infusion force. For example, low-friction sleeves or coatings may be applied over the stiffer outer tube portion of the inner fluid lumen assembly without affecting the inner flexible lumen that is selected to reduce adsorption of viral particles. In other embodiments, a friction-reducing jacket may be applied over a laser-cut tube outer shell while preserving the tube's variable stiffness characteristics, and the sheath ID clearance and/or texturing is selected to provide a balance of low actuation force and adequate column support during puncture. In still further embodiments, the friction reduction features described herein reduce the force required to actuate the inner fluid lumen assembly, thereby improving one-handed operation and providing more predictable distal needle motion while maintaining compatibility with the endoscope working channel and the safety features (including fluid stop mechanisms) described elsewhere in the present disclosure.

### Dual-Line Workflow

While in some embodiments, a single line workflow with a lumen has been described, in other examples, a dual-line workflow may be implemented. In some embodiments, a dual-line workflow can allow for a dual-fluid process, such as for priming and then for therapy. Therefore, in an example embodiment of the present disclosure, the system supports a dual-line (two-fluid) workflow specifically for priming/verification followed by therapy.

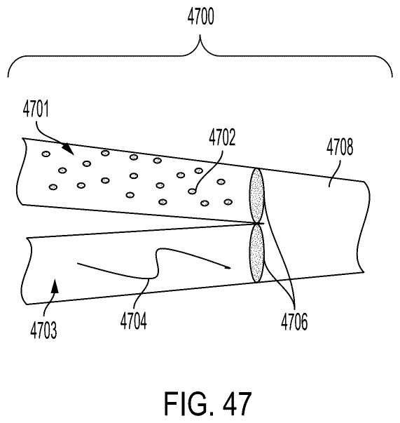

FIG. 47 shows an example portion 4700 of a dual-line workflow assembly. In some embodiments, the device includes two fluid paths 4701 and 4703 within a working-channel-compatible footprint, such as a coaxial dual-lumen tube, side-by-side micro-lumens within a common jacket, or a proximal dual-channel manifold that merges to a single distal lumen 4708 with staged delivery. The staged delivery may be accomplished by controlling flow of the fluid within each fluid path 4701 and 4703, such as by using valves 4706.

In further example embodiments of the present disclosure, the staged delivery between the first fluid path 4701 and the second fluid path 4703 may be accomplished using one or more flow control elements including, but not limited to, valves 4706 and/or separate upstream actuation sources. For example, in some embodiments the system includes a first actuator configured to drive a first fluid (e.g., a priming or verification fluid) through fluid path 4701 and a second actuator configured to drive a second fluid (e.g., a therapeutic composition) through fluid path 4703, such that the staged delivery is achieved by selectively energizing the first actuator and the second actuator in a predetermined sequence, optionally with the fluid paths remaining open or including passive check valves. In other embodiments, the system employs a single actuator to generate pressure or displacement and uses one or more active valves (e.g., pinch valves, rotary selector valves, micro-solenoid valves, or other occlusion/selection mechanisms) to route the driven fluid into the selected fluid path 4701 or 4703, or to alternately connect a common actuator to separate fluid reservoirs that feed the respective paths. In still further embodiments, the system includes a pressure source (e.g., pneumatic pressure, peristaltic pumping, or a syringe drive) coupled with a valve manifold configured to switch between a first reservoir and a second reservoir, thereby enabling staged delivery without requiring two independent pumps. In each such embodiment, the system may be configured to prevent cross-contamination and unintended mixing between the fluids (e.g., by including check valves, purge volumes, dead-volume minimization features, or isolation segments), and the control sequence may be executed manually by the operator or automatically by a controller that coordinates actuator operation and/or valve states to deliver a verification bolus followed by a therapeutic infusion.

In further embodiments, the system executes a control sequence including: deliver a brief contrast bolus to confirm tip location and outflow; switch to therapy for the main infusion; and optionally repeat verification at subsequent sites. In some embodiments, the dual-line configuration is implemented with minimal distal complexity by placing the switching mechanism proximally, such that a single distal needle lumen is shared while two proximal sources are selectable. In other embodiments, truly separate distal lumens terminate at distinct outlets (e.g., separate ports) to allow different flow profiles, different delivery depths, and/or different dispersion behaviors. In further embodiments, the system is configured to flush between modes to control dead volume and to maintain known concentrations at the distal outlet.

In an example embodiment of the present disclosure, the system uses microbubble contrast for immediate feedback of outflow and needle placement. In some embodiments, microbubbles 4702, shown in FIG. 47, are administered to enhance contrast harmonic imaging (CHI) such that a brief bolus reveals the location of the needle tip and/or early dispersion behavior before a therapeutic infusion with therapeutic infusate 4704 is delivered. Such feedback may allow the operator to detect undesired outflow pathways, to identify poor coupling with the target tissue compartment, and/or to reduce the probability of delivering a full dose at a suboptimal site.

In some embodiments, microbubble use is implemented as a dual-fluid workflow with a priming/verification phase followed by a therapy phase. In one example embodiment, the device includes two proximal channels (or two fluid paths) that converge at or near the distal needle such that a first fluid (e.g., microbubble contrast 4702) can be delivered briefly and then switched to a second fluid (e.g., therapeutic infusate 4704), such as shown in FIG. 47. In such embodiments, a valve manifold, pinch valves, and/or a multi-port selector may be integrated into a proximal handle attachment or reusable module, with the distal disposable segment maintaining a compact footprint compatible with the working channel.

In other embodiments, microbubbles are co-formulated with the therapeutic infusate upstream (e.g., at a pharmacy or preparation step) such that only a single lumen is required in vivo. In such embodiments, the system may be configured to deliver a low-volume “leading front” of microbubbles at the start of each infusion site, optionally by controlling a concentration gradient, a prefilled segment, or an upstream mixing protocol. In still further embodiments, the system compensates for dead volume by delivering a predetermined priming volume and then initiating CHI observation at a timed interval to ensure that microbubbles reach the distal needle before interpreting the contrast signal.

### Drive Mechanism

The drive mechanism described herein is similarly versatile. In some embodiments, the force applied at the syringe is in the range of approximately 0.75-2.5 lbf, with the understanding that the specific force translates to pressure based on the syringe barrel's internal diameter. Examples include designs with different force ranges, which may vary from as little as 0.05 lbf up to 30 lbf or more, to meet different flow rate and pressure requirements. Examples include devices with internal drive mechanisms, small handle profile, and low weight, with all representing novel features for devices of this type and use. Actuation of this drive mechanism occurs via a user-operated button, coupled with a latch-and-stop mechanism enabling precise dosing or multiple sequential infusions from the same medicament container. Additionally, a safety stop mechanism associated with the button prevents inadvertent or accidental actuation, ensuring controlled and intentional infusion sequences.

According to some embodiments, the system further includes a hand-held infusion pump device configured to precisely control the delivery of treatment materials into the patient. The hand-held infusion pump can be configured to securely hold a medicament container, such as a syringe, and to accurately control the pressure inside the syringe via controlled force application to a plunger of the syringe. By precisely controlling the applied force and travel of the plunger, the infusion pump device achieves highly reproducible delivery rates, pressures, and volumes of medicament as it drives the therapeutic material from the syringe into the fluid lumen and ultimately into the tissue through the distal infusion needle.

According to further embodiments, the hand-held infusion pump can include an integrated mechanism configured for performing multiple sequential infusions from a single medicament container. In some examples, an initial actuation (such as pressing a button) initiates infusion by releasing a drive mechanism to apply controlled pressure to the syringe plunger, thus driving medicament through the needle. The mechanism can incorporate one or more stop positions that automatically halt advancement of the drive mechanism at predefined intervals, facilitating controlled partial infusions or sequential injections. Subsequent infusion(s) at either the same or different locations, or subsequent doses at the same site, can be performed by releasing the drive mechanism again—for example, by re-pressing the initial button or activating a separate control element. Additional configurations include adjustment mechanisms to select from multiple potential stop positions, enabling the operator to predetermine specific dosing increments or number of infusions from a single syringe. Moreover, the pump can feature selectable mechanisms allowing operators to preset and administer a precise dose of medicament with each infusion, thereby improving dosing accuracy and procedural efficiency.

### Adjustment and Positioning Mechanisms

### Needle Depth Adjustment

Since the pancreas and surrounding anatomical features differ in size by patient, the device according to at least some embodiments provides a variable length of needle exposure. The length of the exposed needle may be adjusted using an adjustment mechanism of the handheld device. The needle may be ejected from a surrounding sheath by an amount set by the medical professional, for example after viewing the subject's pancreas using ultrasound imagery. The needle may also be retracted into a surrounding sheath between infusions.

The handheld infusion system in some embodiments further includes an adjustment assembly disposed at the distal end of the handle, facilitating precise positional control of both the outer sheath and the inner fluid lumen. A sheath adjustment mechanism enables selective positioning of the sheath relative to an attached endoscope, allowing operators to set and adjust the amount of sheath length extending distally beyond the endoscope channel. Additionally, a needle depth adjustment mechanism provides a selectable distal stop for the handle body, thus enabling accurate and reproducible control over the maximum penetration depth of the infusion needle into targeted tissue as also described previously herein. The handle body advances distally toward this stop position, correspondingly advancing the infusion needle from a retracted position within the sheath into the targeted tissue. In some examples, the needle adjustment is between 0 and 6 cm and the sheath adjustment enables 5 cm of length change (e.g., 137.5 cm to 142.5 cm of sheath length extending beyond the coupling, in a representative example). In other examples the adjustment range can be larger, for example between 0 cm and 8 cm, or between 0 cm and 10 cm or more.

In some embodiments, the hand-held infusion pump device also includes an integrated mechanism to adjust and set the needle penetration depth. This depth-setting mechanism establishes a stop position for distal advancement of the infusion pump device, which is fixedly coupled to the inner fluid lumen and distal infusion needle. By adjusting the position of this stop, the operator can predetermine and limit the maximum penetration depth of the needle into targeted tissue. When the infusion pump device is advanced proximally towards the stop position, the needle extends distally from the outer sheath up to the predetermined depth. This ensures consistent needle penetration, minimizes risk of tissue damage or inadvertent puncture beyond the intended target, and improves the repeatability of infusions across multiple treatment sites.

In additional embodiments, the hand-held infusion pump device further incorporates an integrated adjustment mechanism to selectively alter and set the length of the exterior sheath between an endoscope coupler and the proximal end of the sheath, which is fixedly secured to the infusion pump device. This mechanism allows precise positioning of the outer sheath relative to the endoscope to which the system is coupled, thereby providing improved operator control and enhanced accuracy in positioning the infusion assembly for different EUS-guided procedures.

In another example embodiment of the present disclosure, the device supports semi-automatic or automatic needle advancement, with programmed insertion and safety thresholds. In some embodiments, a motorized drive is added to the handle portion to advance/retract the needle relative to the sheath under electronic control, optionally retaining manual override. The motorized drive may include a lead screw, gear train, rack-and-pinion, linear motor, or other linear actuator that couples to the existing carriage or drive mechanism that moves the needle, including, for example, pneumatic or hydraulic actuators.

In some embodiments, programmed insertion includes a sequence in which the needle advances to a planned extension derived from stop/slider settings and/or EUS-derived depth estimates, pauses for a verification step, and then proceeds to infusion if safety checks are satisfied. Safety thresholds may include force thresholds (axial and/or lateral), flow thresholds, and imaging thresholds. In further embodiments, the system includes an emergency stop behavior that immediately retracts the needle into the sheath upon detection of a “red-zone” condition, as described further herein in relation to needle force sensing, or a user command.

### Rotatable Tool Mount

Furthermore, in some embodiments the system includes a selectively lockable rotational coupling for ergonomic and precise device orientation relative to the endoscope. That is, aspects of the present technology provide a rotatable tool mount configured to mount a medical tool to an endoscope in a manner which allows rotation of the tool relative to the endoscope after the two are tightened together. The tool may include, for example, an infusion system for infusion of a gene therapy composition into a target anatomical feature, such as the pancreas. The infusion tool may include a lumen configured to transport the gene therapy composition from a vial on a handle of the tool to a needle on a distal end of the lumen. The tool may be mounted to the endoscope by screwing the two together. However, after they are screwed together, the handle of the tool may be facing a direction the medical professional does not prefer. For example, if the handle of the tool is asymmetrical, the medical professional may have a preferred orientation of the handle. A rotatable tool mount permits the medical professional to rotate the handle relative to the endoscope after the tool and endoscope are fixedly attached. The medical professional may thus ensure that the tool handle is at a desired angle for purposes of comfort, ease of use, effectiveness of use, or for other reasons.

Additional optional device features can further enhance usability, precision, and operator convenience. For instance, in some embodiments, the system includes a selective lock mechanism positioned at the coupler between the infusion pump device and the endoscope. This selective lock enables the infusion pump device to rotate relative to the endoscope, thus facilitating more ergonomic positioning, improved device maneuverability, and enhanced targeting flexibility during the procedure.

### Safety and Usability Features

### Fluid Stop Mechanism

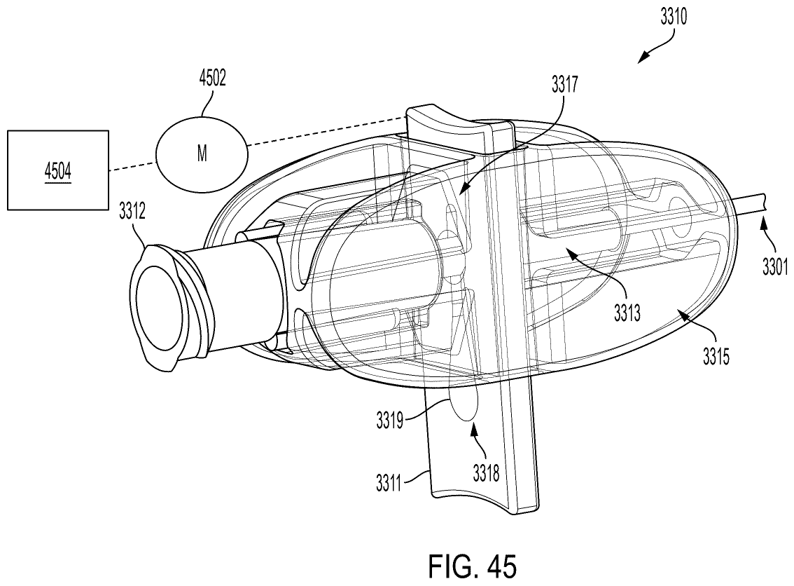

The handheld infusion system in some embodiments incorporates several safety and usability features. A fluid stop mechanism, which may serve as an emergency stop mechanism, may be positioned proximally on the inner fluid lumen assembly and permits immediate occlusion or reopening of fluid flow between the medicament container and infusion needle. This mechanism facilitates rapid pausing of infusions and helps maintain lumen priming when connecting or disconnecting fluid sources as well as helps ensure that the medicament (e.g., virus) does not leak when the needle is repositioned between infusions. Moreover, the fluid stop mechanism may permit fine control over the amount of fluid delivered to the subject, and thus may facilitate the performance of multiple infusions using a single cartridge of the gene therapy composition. The fluid stop may be operated between infusions to prevent leakage from the needle tip between performing infusions at separate locations of the pancreas or other anatomical feature. The fluid stop mechanism may be motorized as described in relation to FIG. 45 herein.

In certain embodiments, the system incorporates a fluid stop positioned along the fluid lumen between the medicament container and the inner fluid lumen leading to the distal needle. This fluid stop allows operators to rapidly pause the infusion mid-procedure, immediately halting medicament flow as needed. This feature can serve as an emergency feature to rapidly stop flow of fluid in case of an emergency. However, more generally, the fluid may be stopped between infusions to prevent leakage or may be stopped to provide multiple infusions from a single cartridge rather than a single infusion. Furthermore, this fluid stop can also be employed to maintain lumen priming during connection or disconnection of the medicament container from the fluid lumen, thus preventing air entrainment or fluid loss. A lock mechanism, positioned upstream and in proximity to this fluid stop, can further facilitate safe and efficient management of priming fluid and medicament during device setup and operation.

In another example embodiment of the present disclosure, the fluid stop mechanism is additionally or alternatively configured to operate as a flow regulation mechanism, rather than solely as an emergency stop mechanism. In such embodiments, the fluid stop includes a controllable restriction feature (e.g., a variable orifice, adjustable pinch region, valve seat, or deformable constriction section) having a selectable constraint area that is set to reduce and/or stabilize volumetric flow rate for a given upstream driving pressure generated by the infusion mechanism. By providing a controlled restriction, the fluid stop may be used to achieve a desired infusion flow profile (e.g., lower peak flow, reduced ramp rate, and/or reduced sensitivity to pressure transients) while still preserving the ability to rapidly occlude the fluid pathway when commanded.

In certain embodiments, the fluid stop mechanism comprises a clamping element that compresses a flexible segment of the inner fluid lumen to define an effective orifice area, wherein the clamping element is operable over a continuum of intermediate positions between fully open and fully closed. In such embodiments, a user-adjustable actuator (e.g., a thumbwheel, lever, detented slider, or calibrated dial) or a motorized actuator (e.g., micro-motor, stepper, solenoid with controlled stroke, or shape-memory actuator) sets the constriction to a predetermined level. The constriction level may be selected manually or automatically by a controller, and may be maintained during an infusion to regulate flow rate and/or reduce pulsatility associated with a pressure-driven infusion source.

In some embodiments, the system is configured such that a common pressure-driven infusion mechanism can be used with therapeutics having different viscosities, with the fluid stop mechanism serving as a compensatory flow regulator. For example, a first therapeutic formulation having a lower viscosity may be delivered under a first pressure condition, while a second therapeutic formulation having a higher viscosity may be delivered under the same pressure condition but with the fluid stop set to a different effective orifice area to achieve a comparable target flow rate and/or total delivered volume over a similar infusion interval. In this manner, the device may reduce the need to modify the upstream pressure generator across different medicaments, while maintaining predictable delivery performance.

In further embodiments, the device includes one or more sensors (e.g., a pressure sensor upstream and/or downstream of the fluid stop, a flow sensor, or a motor position encoder) that enable closed-loop or semi-closed-loop flow control using the fluid stop mechanism. For example, the system may estimate flow rate based on pressure differential across the fluid stop and a stored calibration curve relating constraint area to flow for a given fluid viscosity range, and may adjust the constraint area in real time to maintain a target flow rate or to enforce a maximum allowable flow. In other embodiments, the system implements a look-up table or algorithmic model that selects a fluid stop setting based on a user-entered medicament identifier, measured temperature, or measured viscosity proxy, thereby providing standardized delivery across different formulations.

In still further embodiments, the flow regulation function of the fluid stop is used to reduce risk of tissue injury, leakage, or undesired bolus buildup by limiting instantaneous volumetric flow rate at the needle and/or smoothing transient pressure spikes when the needle is positioned in tissue. For example, the controller may initiate infusion in a “soft-start” mode in which the fluid stop begins at a more restrictive setting and gradually opens toward a less restrictive setting over a ramp period, thereby reducing the likelihood of sudden high-flow jets and improving control of dispersion. Additionally, the fluid stop may be configured to revert rapidly from any intermediate flow-regulation position to a fully occluded position upon receiving an emergency stop command, preserving the emergency stop utility while enabling fine flow regulation during routine operation.

In certain embodiments, the fluid stop mechanism is positioned proximally on the inner fluid lumen assembly as described herein, and the same mechanism is used in multiple modes, including (i) an emergency stop mode (fully occluded), (ii) an open mode (minimal restriction), and (iii) one or more regulated-flow modes (partial restriction). In some embodiments, the regulated-flow modes are associated with discrete detents corresponding to calibrated effective orifice areas, enabling repeatable flow settings across procedures and operators. In other embodiments, the regulated-flow mode is continuous and may be commanded by a motorized actuator and monitored via actuator position feedback, thereby enabling the fluid stop mechanism to function as an adjustable flow regulator compatible with varying therapeutic viscosities while operating with the same pressure-driven infusion mechanism.

### Magnetic Attachment and Friction Control

Further optional features of the infusion system include a magnetic attachment system integrated into the handle's distal region, magnetically securing the handle body to the depth adjustment mechanism when the needle is fully advanced, thereby resisting unintended needle retraction during positioning or prolonged infusions. A friction mechanism, such as a friction roller, may also be incorporated into the handle to provide additional controlled resistance against unintended needle movement and can be tunable by selecting different tubing material and geometries.

According to another embodiment, the needle depth-setting mechanism can feature a magnetic coupling attachment between the hand-held infusion device and the stop mechanism. With the needle fully extended to its predetermined penetration depth, this magnetic coupling provides sufficient attraction force to securely hold the infusion device in position relative to the stop, thereby preventing inadvertent withdrawal or retraction of the needle during delicate positioning or during extended infusion periods. This magnetic attachment thus improves procedural stability, reduces operator fatigue, and enhances accuracy during infusion.

Further, some embodiments integrate a friction-based movement-control mechanism into the hand-held infusion device. This friction mechanism is configured to control the advancement or retraction of the infusion device relative to the set stop, thereby reducing unintended needle movements during positioning, infusion, or aspiration procedures. Adjustable friction settings enable operators to fine-tune device responsiveness and tactile feedback according to their preferences, procedural requirements, or anatomical considerations, contributing to safer and more controlled tissue penetration.

### Closed-Loop Infusion Safety Control

In another example embodiment of the present disclosure, the system includes closed-loop infusion safety control driven by EUS cues.

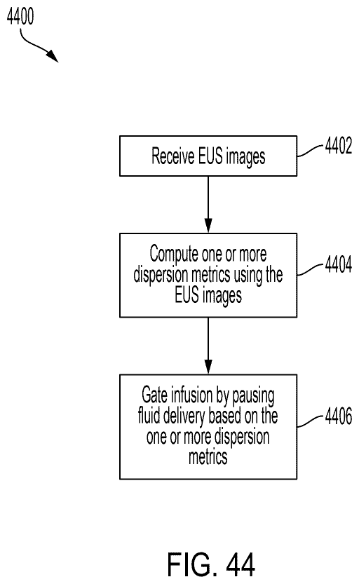

FIG. 44 is a flowchart illustrating a method 4400 of controlling an infusion procedure, according to some embodiments. The method 4400 may include an act 4402 of receiving EUS images, as described herein. The method may further include an act 4404 of computing one or more dispersion metrics using the EUS images from act 4402. The method may include an act 4406 of gating infusion by pausing fluid delivery based at least in part on the one or more dispersion metrics computed in act 4404.

In some embodiments, as described in relation to FIG. 44, the system receives EUS images and computes one or more dispersion metrics, such as an area of hyperechoic change, a contrast-enhanced region boundary, a spread rate, a spread-direction metric, and/or a proximity-to-boundary metric. When the dispersion metric indicates that sufficient spread has been achieved (or that spread is becoming unsafe), the system automatically gates infusion by pausing or stopping fluid delivery.

In some embodiments, infusion gating is achieved by actuating a motorized occluder or pinch mechanism coupled to the baseline device's fluid stop structure. For example, the baseline clamp/occlusion feature may be fitted with a small actuator (e.g., a micro-motor, solenoid, SMA element, or piezo clamp) that drives the occluder into an occlusion region to rapidly close the fluid path. In other embodiments, the system is configured to arm/disarm an electronic latch such that infusion can only proceed while one or more safety conditions remain satisfied.

In further embodiments, the closed-loop control includes explicit conditions tied to mechanical occlusion events and clinical prompts. For example, when a dispersion threshold is met, the system may: (i) stop infusion; (ii) issue an instruction to retract the needle into the sheath; and (iii) display a suggested next target location based on remaining coverage needs. In still further embodiments, the system uses CHI-specific cues (e.g., microbubble spread as described in relation to FIG. 47) to separate “tip confirmation” from “therapeutic dispersion,” thereby improving robustness.

In another example embodiment of the present disclosure, the system includes a comprehensive closed-loop infusion safety control framework that explicitly ties imaging-derived thresholds to mechanical occlusion events. In some embodiments, the system monitors dispersion in real time and, upon meeting a threshold (e.g., sufficient spread, undesired boundary proximity, rapid off-target spread), commands a motorized clamp to occlude the fluid line. In further embodiments, the system records the time, infusion volume estimate, drive state, and dispersion metrics at the moment of occlusion to support post-procedure documentation and iterative optimization.

In another example embodiment of the present disclosure, the system includes an integrated “arming” and “gating” state machine. In some embodiments: a first state requires confirmation of imaging landmarks and stable positioning; a second state allows needle extension; a third state allows infusion; and a fourth state enforces retraction and/or repositioning.

Transitions between states can be driven by sensor inputs (e.g., stop position, sheath position, force, flow), user inputs (e.g., button presses), and imaging-derived metrics (e.g., virtual needle within corridor). In further embodiments, the system provides a guided workflow that reduces cognitive burden and improves repeatability, including prompts such as “confirm landmark set,” “confirm stability dwell,” “advance enable,” “infuse enable,” “stop/retract,” and “move to next target.”

### Needle Force Sensing

In another example embodiment of the present disclosure, the device includes needle force sensing configured to maintain needle loading within an acceptable “green” range (“green zone”) and to provide warning (“yellow”) and stop/withdraw (“red”) feedback when force exceeds one or more thresholds (entering yellow or red zones, respectively).

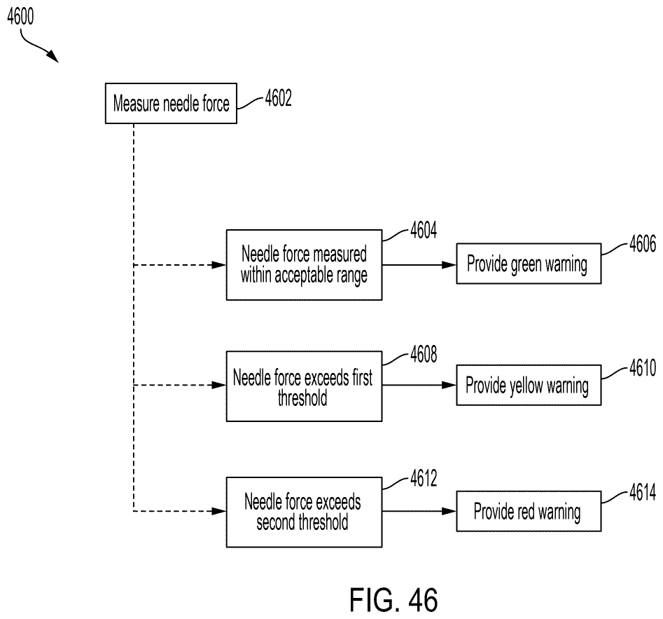

FIG. 46 shows an example needle force sensing procedure 4600. In the force sensing procedure 4600 as shown in FIG. 46, needle force may be measured in act 4602 as described further below. If the needle force is measured within an acceptable range as in block 4604, a green warning is provided as in act 4606. If the needle force is measured as a value exceeding a first threshold as in block 4608, a yellow warning is provided as in act 4610. If the needle force is measured as a value exceeding a second threshold as in block 4612, a red warning is provided as in act 4614. There may be additional threshold comparison blocks with third, fourth, or greater thresholds, as the first and second thresholds are non-limiting examples. The warnings may be provided using other indicia such as other color-based, text-based, or audible warnings.

In some embodiments, force sensing includes detection of lateral load (e.g., bending or side loading), detection of axial load (e.g., insertion force), and/or detection of transient spike events (e.g., sudden lateral load spikes indicating snagging, slipping, vessel wall contact, or hard-structure contact). Such measurements can be beneficial because off-axis loading may indicate unsafe needle manipulation, increased tissue trauma risk, kinking, and/or an increased probability of unintended movement of the needle tip relative to the target tissue during infusion.

In some embodiments, needle force sensing is implemented in a hub region proximal to the needle shaft, such as within or adjacent to a nose cone, coupling, or transition region between a rigid tube (e.g., hypotube/laser-cut tube) and a flexible lumen segment. For example, strain gauges, thin-film piezoresistive elements, capacitive strain sensors, optical strain sensors (e.g., fiber Bragg grating), or impedance-based strain sensing traces may be applied to or embedded within a structural member mechanically coupled to the needle such that bending and/or axial loads are transduced into an electrical signal. In other embodiments, force sensing is implemented along a portion of the needle shaft (e.g., a proximal needle segment that remains within a protected housing) such that lateral deflection causes a measurable strain signature without requiring sensors at the distal tip.

In further embodiments, the system uses force sensing to trigger one or more safety behaviors. Non-limiting examples include: providing haptic feedback through the handle when lateral load exceeds a threshold; automatically pausing infusion (e.g., by actuating a fluid stop) when lateral load increases rapidly (suggesting slippage, snagging, or contact with a harder structure); prompting the operator to retract the needle into the sheath when measured lateral load exceeds a “red-zone” limit as described above; limiting further extension of the needle unless a “confirm” action is received; and/or automatically retracting the needle into the sheath upon detection of a red-zone event, optionally with a manual override.

In still further embodiments, the system logs force (e.g., needle force) signatures across multiple infusion sites to identify patterns associated with successful infusion and/or adverse events, thereby enabling procedure standardization, training feedback, and/or post-procedure documentation. In some embodiments, the logged signature is time-aligned with EUS frames and device-state data to enable correlation between operator actions, tissue response, and image-based dispersion metrics.

### Flow Sensing

In another example embodiment of the present disclosure, the device includes flow sensing to monitor flow as described herein, and also to generate actionable information regarding nominal versus off-nominal infusion behavior. In some embodiments, the flow sensing is configured to confirm a nominal “start profile” at the onset of infusion (e.g., a predictable ramp in flow rate after a clamp is opened or an actuator is engaged), to detect occlusion or partial occlusion (e.g., due to blocked side opening, tissue compression, or kinking), to detect leak conditions (e.g., upstream disconnection or unintended fluid diversion), to detect backpressure-related anomalies (including rising pressure at constant drive force), and/or to detect bubble/air passage.

In some embodiments, flow sensing is implemented via an inline sensor integrated into the disposable flow path (e.g., a micro-turbine, thermal flow sensor, ultrasonic transit-time sensor, differential pressure element, or Coriolis micro-sensor). In other embodiments, flow sensing is implemented as a non-contact sensor within a reusable attachment coupled to the handle, such that a transparent section of tubing passes through an optical or infrared measurement zone. In one non-limiting example, an IR emitter/detector pair measures pulsation, bubble passage, and/or optical density changes correlated with flow, allowing the sensing electronics to remain in the reusable module while preserving a simple disposable tubing path.

In further embodiments, the system uses flow sensing to trigger an “E-stop” behavior, either automatically or by prompting the operator. For example, if measured flow deviates from an expected range for a given commanded infusion state (e.g., clamp open and syringe drive engaged), the system may automatically actuate a motorized occluder and present a prompt instructing the endoscopist to pause and reposition the needle. In still further embodiments, the system distinguishes among failure modes (e.g., no-flow due to occlusion, reduced flow due to partial blockage, excessive flow due to leak/disconnection) based on the combination of flow signals, upstream pressure signals, drive state, and/or device-state signals.

### Infusion Gating

In another example embodiment of the present disclosure, the system implements “green-light” infusion gating with ultrasound overlays and device-state sensing. In some embodiments, an operator (or algorithm) may be required to confirm a checklist of anatomic landmarks on EUS (e.g., a duct location, vessel clearance, capsule boundary, and target parenchyma features) before infusion is armed. In further embodiments, the system displays a projected needle trajectory and expected extension based on stop/slider/sheath position sensing, optionally combined with EM tracking, as described further herein, thereby providing a virtual representation of where the needle will go if advanced.

In some embodiments, the system generates an alarm or lockout if the projected path intersects a no-go region or drifts outside a safety corridor. For example, the system may define a keep-in corridor that accounts for acceptable depth and angle margins relative to segmented anatomy, and may only arm infusion when the virtual needle remains within the corridor. In still further embodiments, the system may require the operator to hold a stable pose for a predetermined time window (e.g., “stability dwell”) before the device permits needle extension beyond the sheath.

### Clinical Application: Pancreatic Infusion

### Target Anatomy and Infusion Strategy

In some embodiments, treatment of a subject with the gene therapy composition may involve multiple infusions into the subject's pancreas. The target cells of the pancreas (e.g., the Beta cells) may be distributed throughout the pancreas, such that performing multiple infusions may increase the effectiveness of the gene therapy. The pancreas is characterized by a higher concentration of Beta cells in the tail. Thus, in at least some embodiments, an infusion of the gene therapy composition may target the tail of the pancreas. An additional infusion may be performed in the body of the pancreas. In some embodiments, further infusions may be performed.

Infusing into the tail and body of the pancreas may be performed via the subject's stomach. That is, the infusion system may be configured to puncture through the stomach wall into the pancreas. Thus, the infusion system may provide a needle of sufficient length to make the puncture from the stomach into the pancreas.

### Expanded Clinical Applications

In additional embodiments of the present inventive concepts, the systems, devices, and methods described herein can be adapted for an expanded range of therapeutic and diagnostic applications beyond those previously disclosed for the treatment of diabetes or related metabolic disorders. By refining and customizing elements such as the infusion assembly, syringe assembly, drive assembly, and associated functional components, the disclosed device can be employed in diverse Endoscopic Ultrasound (EUS)-guided and percutaneous interventions targeting multiple organ systems.

According to some embodiments, the system can be adapted for various EUS-guided fluid delivery procedures. Such procedures can include the targeted infusion of gene therapy agents, organoid delivery, diagnostic contrasts, regenerative medicine treatments, immunotherapies, or other biologics into one or more organs, including the pancreas, liver, bile ducts, gallbladder, lymph nodes, adrenal glands, stomach, and intestines. For example, to access deeper tissue planes or difficult-to-reach organ regions, the infusion needle length may be increased from approximately 6 cm to 8 cm or more although in other embodiments the infusion needle length may have a maximum extension from a surrounding sheath of approximately 6 cm. Where high-viscosity therapeutic materials (e.g., certain gene therapy formulations) are delivered, flow rates can be slowed, or pressure settings can be carefully modulated, to ensure safe and effective deposition while minimizing tissue trauma or backflow.

### Injection and Targeted Therapy Delivery

In some embodiments, the system can be employed for EUS-guided fine-needle injection (FNI) procedures that deliver chemotherapeutic agents, immune checkpoint inhibitors, or other targeted treatments to malignant tissues. These tissues may include pancreatic tumors, liver metastases, gastrointestinal lesions, adrenal masses, or lymph node metastases. The system can also be configured for delivering regenerative therapies—such as stem cells or growth factors—to diseased or damaged tissue regions for functional restoration. The infusion assembly and depositing device can incorporate features to maintain sterility, track delivered volumes precisely, and ensure consistent dosing for each targeted site.

In additional embodiments, the system can facilitate submucosal injection of fluids into the stomach or intestines to create a cushion beneath the mucosal layer for resection or other endoscopic procedures (e.g., endoscopic mucosal resection (EMR) or endoscopic submucosal dissection (ESD)). A specialized needle tip, such as a beveled or angled design, can improve fluid dissemination while preventing inadvertent puncture beyond the submucosal plane. Similarly, a suitable coating or surface treatment can be applied to the needle shaft to reduce friction, improve visualization under ultrasound, and minimize leakage of injected materials.

### Aspiration

In certain embodiments, the system can be configured for EUS-guided aspiration of fluid, cystic contents, or other materials from the pancreas, liver, gallbladder, bile ducts, lymph nodes, adrenal glands, stomach, or intestines. The drive assembly, which may feature a constant force spring, can be reoriented or redesigned to apply a pulling force on the syringe plunger rather than the typical pushing force. Needle diameter can be increased (e.g., from 27 G to 25 G or 22 G) if higher flow rates are required or if more viscous fluids are to be aspirated. This aspiration capability is particularly beneficial for the evaluation and drainage of pancreatic pseudocysts, hepatic cysts, or other fluid-filled collections within the abdomen.

### Biopsy Collection

In further embodiments, the system can be adapted for biopsy collection. The needle tip can be changed from an anti-coring or conical design (optimal for fluid delivery) to a coring, bevel-type needle that facilitates the acquisition of tissue cores. By reversing or modifying the syringe driver to create consistent negative pressure, the system can efficiently perform EUS-guided fine-needle aspiration (FNA) or fine-needle biopsy (FNB). This approach is useful for diagnosing pancreatic masses, submucosal gastrointestinal lesions, lymph node enlargements, hepatic tumors, and adrenal gland lesions. Adjusting the needle diameter to 25 G or larger can increase tissue yield, improving diagnostic accuracy.

### Cyst Drainage

In certain applications, the system can be configured for EUS-guided cyst drainage, such as treating pancreatic pseudocysts, hepatic cysts, or other fluid collections. Here, the system can provide simultaneous drainage of fluid while also administering therapeutic agents (e.g., sclerosants, antibiotics, or sealants) to minimize recurrence and promote healing. Flow- or pressure-controlled delivery features help ensure that these agents are deposited accurately at the target site.

### Ablation Therapies

In some embodiments, the system can be employed for EUS-guided ablation therapies. The infusion assembly may incorporate or be used in conjunction with ablative elements (e.g., radiofrequency ablation (RFA), cryotherapy, photodynamic therapy (PDT), high-intensity focused ultrasound (HIFU), or reversible/irreversible electroporation). After positioning the needle or ablation probe, the system can deliver controlled thermal or non-thermal energy to ablate tumors, pre-cancerous lesions, or hyperplastic tissue in the pancreas, liver, stomach, or esophagus. To enhance treatment outcomes, immune modulators, tumor necrosis factor (TNF)-alpha inhibitors, or other adjunctive agents can be delivered either before, during, or after the ablation procedure.

### Neuromodulation

In further embodiments, the system can be configured for EUS-guided neuromodulation. For example, targeted injections of neurolytic agents or botulinum toxin can be administered to modulate neural pathways implicated in chronic pain, functional gastrointestinal disorders, or metabolic dysregulation. Such applications may include EUS-guided celiac plexus neurolysis or splanchnic nerve blockade to relieve pain associated with chronic pancreatitis or pancreatic cancer. Similarly, miniature electrodes or stimulation leads could be placed to alter neural signaling and manage chronic conditions.

### Additional Procedures

Example system can also be adapted for EUS-guided portal pressure measurements in patients with liver disease, aiding in the assessment of portal hypertension. By carefully positioning a pressure-sensing component in or near the portal vein, the system can measure pressure gradients critical to diagnosing cirrhosis, varices, and hepatic congestion. Additionally, the system can accommodate EUS-guided gallbladder drainage or biliary access procedures, including stent or catheter placement, in patients with obstructive jaundice or other biliary disorders-offering a minimally invasive alternative to percutaneous or surgical interventions.

In some embodiments, the system can be employed in EUS-guided endovascular interventions. For example, targeted delivery of thrombolytic agents, embolization materials, or vascular occlusion devices can be performed to manage gastric varices, splenic artery aneurysms, or gastrointestinal bleeding. The underlying needle or catheter design, alongside flow- and pressure-control features, can be adapted to ensure safe and targeted endovascular access.

In yet other embodiments, the system can be configured for EUS-guided pancreatic duct interventions, enabling the direct injection of antifibrotic agents, anti-inflammatory substances, or enzyme replacement therapies into the pancreatic duct. Such interventions can help manage chronic pancreatitis, ductal strictures, or even early-stage pancreatic malignancies by restoring or maintaining ductal patency and mitigating disease progression.

### System Integration and Advanced Features

### Imaging and Navigational Integration

In all of these expanded applications, the system can be integrated with advanced imaging modalities, pressure sensing, and navigational sensors (e.g., electromagnetic position sensors, fiberoptic shape sensing) to improve real-time visualization of the infusion device's path and the tissue response. The system's console and graphical user interface can display overlayed trajectories or predictive maps of fluid spread, enabling precise targeting of the deposit site and reducing the risk of off-target effects.

In some embodiments, needle visualization may be enhanced with active beaconing and photoacoustic options. In an example embodiment of the present disclosure, the device includes enhanced needle visualization using an active “beacon” configured for momentary localization of the needle under ultrasound imaging.

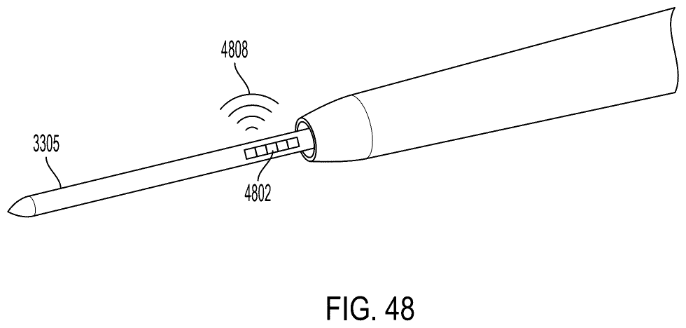

FIG. 48 shows an example distal needle 3305, as described herein, having an active beacon 4802 that is configured to generate a unique signature 4804, according to some embodiments.

In some embodiments, the beacon (e.g., active beacon 4802) is configured to generate a unique signature 4804, such as a distinct ultrasound signature, that is separable from passive echogenic coatings, thereby enabling the operator to rapidly re-acquire the needle tip when the needle is out-of-plane, when acoustic shadowing is present, when the working channel geometry causes a difficult approach angle, and/or when the needle gauge is reduced (e.g., 29-32 G) and passive visibility becomes less robust.

In some embodiments, the active beacon comprises a miniature piezoelectric element mechanically coupled to a portion of the needle and configured to vibrate at or near an ultrasound imaging frequency (or a harmonic/subharmonic thereof) upon command. For example, a piezo ring, strip, or stack may be bonded to a proximal needle segment or a structural member coupled to the needle such that vibration propagates along the needle to create a detectable signature in the EUS field. In other embodiments, the beacon comprises a magnetostrictive actuator, an electrostatic micro-actuator, or another acoustic emitter configured to impart controlled micro-motion.

In further embodiments, enhanced visualization comprises a photoacoustic approach in which optical energy (e.g., delivered via a fiber) produces a transient acoustic emission at or near the needle tip. For example, an absorptive coating, marker, or micro-structure may generate a photoacoustic pulse upon illumination. In such embodiments, the system may be configured for a “locate mode” in which short, intermittent activations produce a visible signature without materially altering the mechanical behavior of the needle, and without requiring continuous activation that could distract the operator.

Further, electromagnetic and/or magnetic tracking can be implemented. In another example embodiment of the present disclosure, the system includes electromagnetic (EM) and/or magnetic tracking configured to determine pose of the needle and to generate an EUS overlay of set depth and expected extension, sometimes referred to herein as a “virtual needle.”

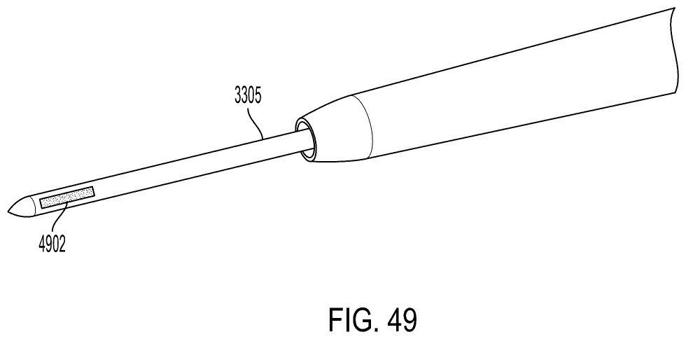

FIG. 49 shows an example distal needle 3305, as described herein, having a magnet 4902 disposed thereon, according to some embodiments. In some embodiments, an EM coil or magnetic marker is disposed on or near the distal end of the needle assembly, on or near the sheath, and/or at a known reference location on the handle or shaft. In other embodiments, the tracking sensor is disposed proximal to the imaging plane but mechanically registered to the needle such that needle extension is inferable via measured device state.

In some embodiments, the baseline depth-adjustment sheath and stop mechanisms are augmented with one or more sensors that indicate device state, such as a slider position sensor, stop position sensor, sheath position sensor, needle extension sensor, and/or rotational position sensor. Non-limiting examples include a potentiometer, Hall sensor with magnet, optical encoder, resistive track, and/or limit switches integrated into the handle and coupled to a moving carriage. Using these device-state signals, the system may compute an expected distal needle extension beyond the sheath and render that expected extension as an overlay relative to the EUS field, optionally together with depth markers, planned depth, safety margins, and/or keep-out zones.

In further embodiments, the system combines EM tracking and device-state sensing to provide real-time guidance and safety alarms. For example, the system may warn if the projected needle path intersects a no-go region (e.g., a vessel, duct, boundary, capsule, or unsafe corridor), may present a lockout that prevents infusion arming until the stop setting corresponds to an acceptable depth, and/or may adjust the overlay depending on whether the operator has moved the stop out of the way versus using it as an active target. In still further embodiments, the overlay includes predicted dispersion zones based on planned infusion volume and tissue model parameters, thereby supporting faster and more consistent site-to-site dosing.

### Functional Enhancements

Additionally, functional elements—such as vacuum, heating, cooling, iontophoretic, or electroporation elements—can be incorporated into the shaft or tip of the infusion assembly to optimize the delivery, uptake, or distribution of the treatment material. For instance, electroporation (e.g., reversible) can enhance cellular uptake of a gene therapy vector following its infusion, while a vacuum or heating element can facilitate fluid withdrawal or tissue ablation, respectively.

The versatility of the system's hardware-including customizable needle gauges, lengths, tip geometries, coatings, and drive mechanisms-supports a broad spectrum of clinical procedures. Fluidic elements can be precisely metered and tracked to limit systemic exposure and reduce toxicity, or conversely, to facilitate controlled systemic distribution if required for certain therapies. For aspiration or biopsy, needle dimension and orientation can be optimized to collect samples with minimal contamination or structural disruption.

### Assistive Operator Features

In some embodiments of the present disclosure, an endoscopic infusion system as disclosed herein is augmented to provide assistive automation that improves procedural safety, efficacy, repeatability, documentation, and/or speed while preserving the fundamental workflow of endoscopic ultrasound (EUS)-guided needle placement and infusion.

By way of non-limiting example, such a baseline system may include: (i) a handle portion that supports controlled advancement and retraction of an infusion needle relative to a surrounding sheath; (ii) one or more depth-setting structures (e.g., a slider, stop, adjustable reference surface, and/or sheath reference); and (iii) a fluid pathway configured to deliver an infusate through the needle. In further embodiments, the baseline system includes a fluid-stop mechanism (e.g., a clamp, pinch, occlusion, or valving feature) configured to interrupt infusion rapidly.

In some embodiments, the baseline system (or a version thereof) is configured for EUS-guided pancreatic infusion of a gene therapy medicament (e.g., viral vector compositions), and includes a small-gauge needle (e.g., about 27 G) with a conical distal tip and at least one side port opening proximal to the tip, with an echogenic coating disposed on an external surface of a distal segment of the needle to increase ultrasonic visibility during EUS imaging. In some embodiments, the conical tip, side-port location, and/or other anti-backflow features are configured to reduce proximal backflow of infusate along the needle track, for example to improve intraparenchymal retention.

In some embodiments, the baseline system includes a self-contained, fully disposable (or mostly disposable) assembly that couples to an endoscope, optionally via an adjustable and/or rotatable coupling, and provides one-handed operation by coupling a proximal handle/drive assembly to the distal needle via a structural member (e.g., a metal hypotube and/or laser-cut tube geometry) that provides sufficient column strength to enable a simple distal movement of the handle portion relative to the endoscope to advance the needle while maintaining depth control. In some embodiments, the baseline system includes a constant-force spring drive (or other compact mechanical drive) that drives a medicament container plunger, enabling repeatable delivery profiles with minimal procedural burden.

In some embodiments, the present disclosure provides two complementary roboticization assemblies. A first assembly can provide add-on assistive and automation features that can be incorporated into the devices and procedures described herein to boost safety, efficacy, and speed. In some embodiments, the first assembly provides sensing, feedback, gating, and/or limited actuation capabilities while preserving the operator-driven EUS workflow. A second assembly can provide fully or semi-robotic workflows (including semi-automatic needle placement, robotic safety interlocks, and image-guided autonomy as described herein).

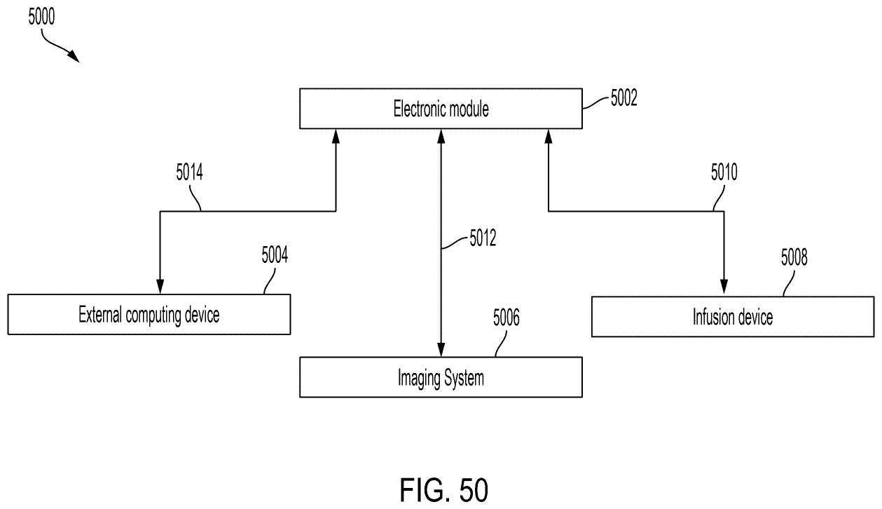

FIG. 50 shows a diagram of a system 5000 that illustrates communication among elements of the system, according to some embodiments. The system 5000 may provide for sensing capabilities described herein, enabling assistive and automation features during an infusion procedure.

In some embodiments of the present disclosure, the system 5000 includes one or more electronic modules 5002 (reusable and/or disposable) that interface with a (baseline) infusion device 5008, as described herein, and communicate with an external computing device 5004 (e.g., a console, tablet, workstation, EUS system, or a dedicated controller). The electronic module may interface with an imaging system 5006, as described herein. While FIG. 50 shows an example system, the system architecture is not so limited, as the respective devices and modules may be in communication with other devices. For example, the imaging system 5006 may be in communication with the external computing device.

The electronic modules, infusion device, imaging system, and external computing device may exchange signals 5010, 5012, and 5014 as shown in FIG. 50. The electronic module(s) may receive signals from sensors disposed on the device, process the signals locally and/or remotely, and generate one or more outputs including: (i) user feedback (visual, audible, or haptic); (ii) gated enablement/disablement of infusion; and/or (iii) actuation of one or more mechanisms such as a fluid occluder, a needle advancement drive, a sheath positioning drive, and/or a stop/slider positioning drive.

In some embodiments, the module architecture is designed to preserve disposability of fluid-contacting and/or patient-contacting components by locating sensing electronics and power systems in a reusable “clip-on” module while leaving a simple disposable tubing path (and/or disposable handle) unchanged.

In some embodiments, the system is configured to interface with an EUS imaging system such that device state and/or safety guidance is displayed as an overlay on an ultrasound image. In other embodiments, the system is configured to receive one or more EUS-derived signals, parameters, or image-derived inferences (e.g., a dispersion metric, a contrast-enhanced region, a vessel proximity metric, a boundary proximity metric, a duct proximity metric, an anatomical landmark confirmation, and/or an out-of-plane risk metric) and to use those signals to control device behaviors including infusion gating, pausing, stopping, retract prompting, and/or reposition prompting.

In some embodiments, the system is configured so that enhancements remain “assistive” and transparent to the clinician—i.e., the clinician remains responsible for interpreting EUS and positioning the needle—while the automation layer and corresponding assemblies add “guard rails,” lockouts, and recording to improve safety and repeatability.

### Virtual Fixtures

In another example embodiment of the present disclosure, the system includes operator-assist “virtual fixtures” for trajectory setup, sometimes described as shared control. In some embodiments, the virtual fixture is computed from EUS-derived reconstruction and defines a safe corridor for needle advancement and infusion, including a keep-in region and one or more keep-out regions. The system may provide haptic guidance through the handle, visual guidance via overlays, and/or discrete lockouts that prevent advancement or infusion if the live projected trajectory exits the safe corridor.

In some embodiments, the virtual fixture interacts with baseline physical stops and sheath mechanisms such that the allowed range of needle extension is constrained both physically and electronically. For example, the device may include an electronically controlled “advance-enable” state that is only active when the stop setting corresponds to an acceptable depth and when the EUS-derived assessment indicates that the tip will remain in target tissue. In further embodiments, the system records fixture violations and correlates them to training guidance, thereby improving procedure standardization.

### Modular Integrations

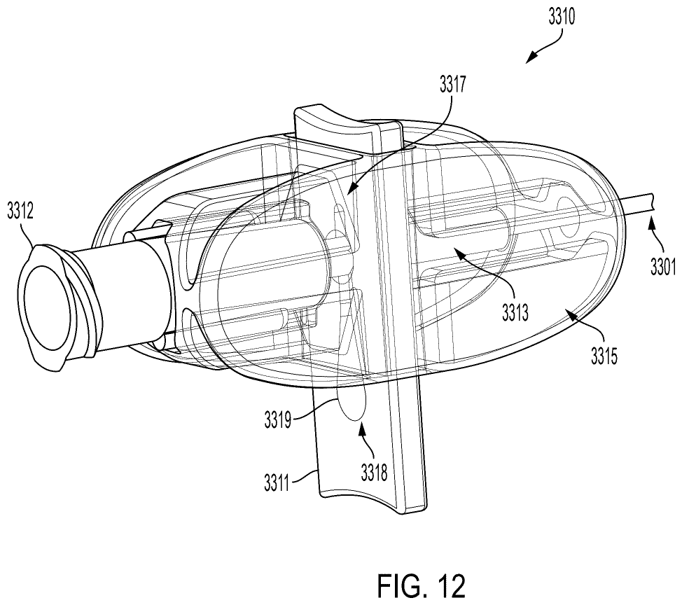

In some embodiments of the present disclosure, any of the above assistive and/or robotic features may be implemented as modular add-ons such that the baseline device remains substantially unchanged in its core mechanical infusion pathway, while additional sensing and actuation are achieved through a reusable handle attachment, an instrumented clamp module, an instrumented stop/slider module, and/or software that interfaces with the EUS system. As a non-limiting example, FIG. 45 shows a motorized fluid stop mechanism. In particular, FIG. 45 shows an example of the fluid stop mechanism 3310 of FIG. 12 with a motor 4502 added on. The motor may be configured to cause motion of the fluid stop mechanism 3310, enabling automation features described herein. The motor may be in communication with a controller 4504, in some embodiments.

Additionally, or alternatively, one or more features (e.g., motorized needle advance, integrated tracking sensors, dual-lumen architecture, steerable needle structures) may be directly integrated during device production while preserving procedural compatibility.

In some embodiments, the electronic module is designed for clinical practicality: minimal added steps, intuitive prompts, deterministic fail-safe behavior (e.g., default-to-occluded on power loss), clean separation between disposable sterile components and reusable electronics, and compatibility with cleaning/sterilization workflows and hospital IT constraints.

### Summary

Collectively, these embodiments provide a versatile, precise, and user-friendly infusion system capable of controlled delivery of therapeutic agents into targeted tissue sites, optimizing both procedural accuracy and patient safety in minimally invasive procedures.

Moreover, in some embodiments, combinations of features are expressly contemplated. For example: closed-loop infusion gating may use both flow sensing and EUS dispersion metrics to differentiate between occlusion and sufficient spread; EM tracking may be combined with device-state sensing to generate a virtual needle overlay with higher robustness; force sensing may be combined with automated retreat and infusion stop to reduce risk during unexpected patient motion; and microbubble verification may be combined with subsequent therapeutic infusion and automatic stop when dispersion reaches a boundary criterion.

In further embodiments, the system described herein may log multimodal data (force, flow, pressure, device state, user actions, and imaging-derived metrics) to enable refinement of thresholds, procedural optimization, training guidance, and post-procedure reporting, while maintaining patient safety interlocks as a primary objective.

Overall, these combined and expanded embodiments illustrate how the present inventive concepts provide a universal platform for minimally invasive, image-guided interventions. By incorporating design modifications specific to different procedure types—such as fluid delivery, aspiration, biopsy collection, cyst drainage, ablation, neuromodulation, stent placement, or endovascular therapy—the system can address a diverse range of medical conditions across various organ systems. This adaptability leverages real-time imaging guidance and sophisticated drive and positioning assemblies to deliver, aspirate, or manipulate fluids and tissues with exceptional precision and safety.

Accordingly, the technology described herein not only addresses the treatment of diabetes and metabolic disorders but also extends to a host of therapeutic and diagnostic procedures, including combinations thereof. The disclosed system enhances clinical outcomes through targeted intervention, reduced invasiveness, and improved operator control. These and other advantages will be more fully understood in view of the following description and the accompanying drawings, which further illustrate and describe certain representative embodiments of aspects of the present technology.

Example devices and enhancements can significantly expand the versatility, procedural efficacy, and clinical applicability of the disclosed technology. By combining robust needle shaft construction, precise infusion pressure control, adjustable needle depth and sheath length mechanisms, sequential infusion capabilities, emergency fluid management features, and complementary operator-focused improvements (e.g., selective locking, magnetic coupling, friction-based controls, and integrated proximal force transmission), the system provides a comprehensive and highly adaptable platform for safe, accurate, minimally invasive, image-guided therapeutic and diagnostic interventions across numerous organ systems and anatomical targets.

### EXAMPLES

### Example 1

Glucagon-like peptide 1 (GLP-1) is a gut hormone that signals satiety and regulates islet hormone secretion. GLP-1 drugs effectively manage diabetes and promote weight loss, but most patients discontinue within a year, leading to weight regain and metabolic decline. A novel pancreatic gene therapy (PGTx) platform was developed for sustained meal-regulated GLP-1 production from beta cells. Here, the potential for PGTx to prevent metabolic disease progression in lean and diet-induced obesity (DIO) mice was assessed.

AAV vector encoding a GLP-1 receptor agonist (Exendin-4) with an insulin promoter for beta-cell-specific expression was developed. Four groups were studied for 5 weeks (wks) (n=8 per group): 1) started on 60% high-fat diet (HFD), treated with single-dose IP PGTx (8.3e11 VGs), and maintained on HFD (DIO PGTx) 2) started on chow diet (CD), given PGTx, and transitioned to HFD at week (wk) 3 (Lean PGTx+HFD) 3) started and maintained on CD (Lean) 4) started on CD and transitioned to HFD at wk 3 (Lean+HFD). Body weight (BW) and blood glucose (BG) were evaluated as disease markers. Data are reported as mean percent change from baseline (BL) or wk 3.

PGTx was well-tolerated. In the DIO PGTx cohort, PGTx reduced BW by 20% at wk 3 and maintained weight loss through wk 5 on HFD (−17%). BW at wk 5 increased by 25% in the Lean+HFD cohort when switched to HFD. However, in the Lean PGTx+HFD group, BW was reduced by 6% at wk 3, and did not rise above BL after the HFD switch. In parallel, PGTx reduced BG by 38% at wk 3 and maintained BG reduction through wk 5 on HFD (−32%). In the Lean+HFD group, BG at wk 5 increased by 20% when switched to HFD. In the Lean PGTx+HFD group, PGTx induced a BG reduction of 15% at wk 3 and prevented HFD-induced hyperglycemia as evidenced by a BG level 9% below BL at wk 5.

These data suggest that PGTx can durably reduce weight and glycemia in DIO and prevent weight gain and hyperglycemia when treatment is initiated before HFD. Lean mice receiving PGTx exhibited less weight loss than their DIO counterparts, suggesting a self-limiting mechanism that could enhance the PGTx the safety profile.

### Example 2. Treatment of Diet-Induced Obesity Mice Using an AAV Vector Encoding a GLP-1 Receptor Agonist

Four groups of C57BL/6 mice were studied for 5 weeks (n=8 per group):
1. HFD/AAV: Mice started on a high-fat diet (HFD), treated with AAV encoding a GLP-1 receptor agonist, and maintained on HFD throughout the study.
2. Chow-HFD switch/AAV: Mice started on chow diet (CD), treated with AAV encoding a GLP-1 receptor agonist, and transitioned to HFD at week 3.
3. Chow control: Mice started and maintained on CD throughout the study (untreated control).
4. Chow-HFD switch/Vehicle: Mice started on CD and transitioned to HFD at week 3 (untreated control).

Body weight (BW) and blood glucose (BG) were evaluated as disease markers throughout the 5-week study period. Blood glucose was measured after a 4-hour fast. Food intake was monitored and calculated in both grams and kilocalories. Data are reported as mean percent change from baseline (BL) or week 3.

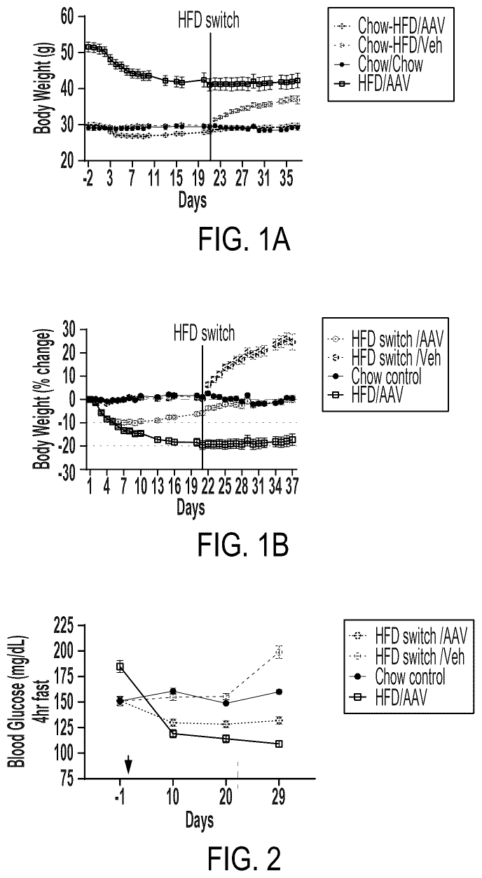

As shown in FIGS. 1A-1B, HFD/AAV mice demonstrated attenuated weight gain compared to untreated mice exposed to HFD. The chow-HFD switch/vehicle group, which transitioned from chow to high-fat diet at week 3 without treatment, showed weight gain following the dietary switch (FIGS. 1A-1B).

The Chow-HFD switch/AAV group, which received treatment while on chow diet before transitioning to HFD at week 3, exhibited resistance to diet-induced weight gain. Despite the transition to high-fat diet, these mice maintained body weights similar to the chow control group maintained continuously on chow diet throughout the study (FIGS. 1A-1B).

Percent change in body weight from week 3 (FIG. 1B) illustrated that the chow-HFD switch/vehicle group experienced weight gain following HFD introduction, while the chow-HFD switch/AAV group showed minimal weight change after the same dietary transition, demonstrating the protective effect of AAV treatment against subsequent diet-induced obesity.

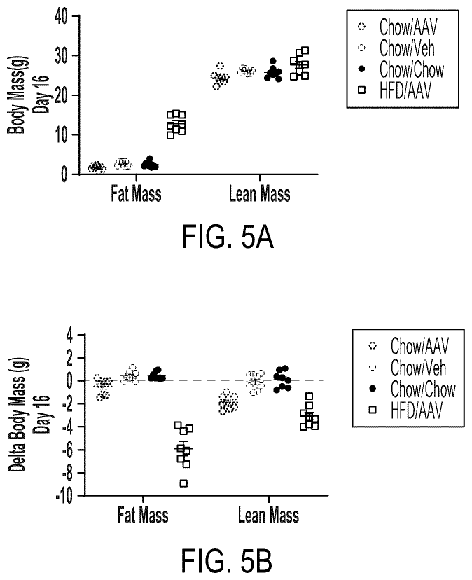

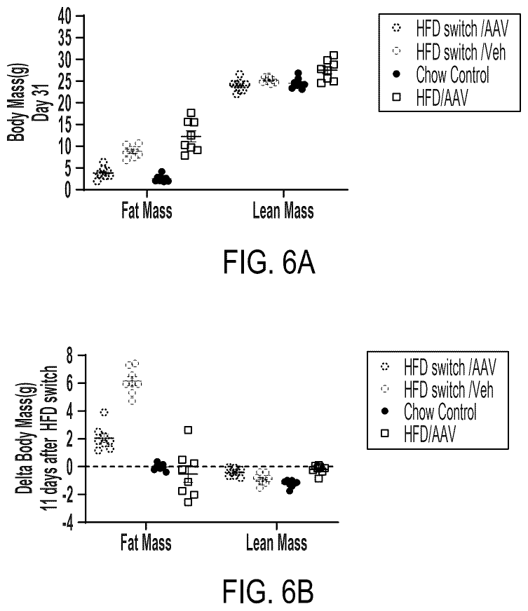

FIGS. 5A-5B show body mass measurements at day 16. Fat mass was highest in HFD/AAV mice at this timepoint. FIGS. 6A-6B show body mass at day 31. The HFD/AAV mice maintained their baseline weight at this timepoint.

FIG. 2 demonstrated the effects of AAV treatment and diet modification on 4-hour fasting blood glucose levels. The chow-HFD switch/vehicle group developed elevated fasting glucose levels following the transition to high-fat diet at week 3. The HFD/AAV group, despite continuous high-fat diet exposure, maintained fasting glucose levels comparable to the chow control group.

The chow-HFD switch/AAV group maintained normal fasting glucose levels even after transitioning to high-fat diet at week 3 (FIG. 2), demonstrating that AAV pretreatment provides protection against diet-induced glucose dysregulation.

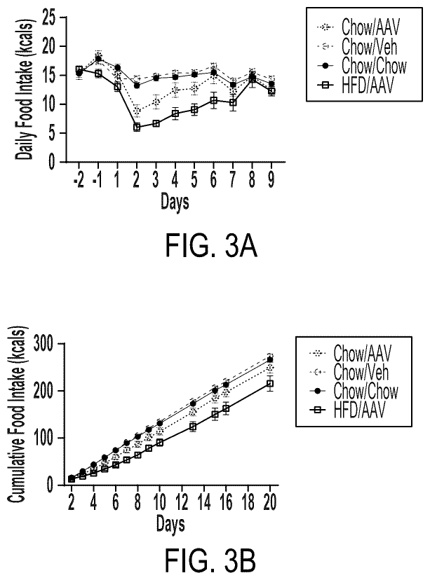

Prior to the dietary transition at week 3, food intake was monitored in all groups. FIGS. 3A-3B show caloric intake during this initial phase. The HFD/AAV group showed reduced caloric intake due to AAV treatment (FIG. 3A). The chow control and chow-HFD switch/AAV groups, both on chow diet during this phase, showed similar food intake patterns.

Cumulative caloric intake over the first 3 weeks (FIG. 3B) demonstrated reduced consumption in the HFD/AAV group compared to untreated mice, indicating sustained reduction in food intake due to AAV treatment.

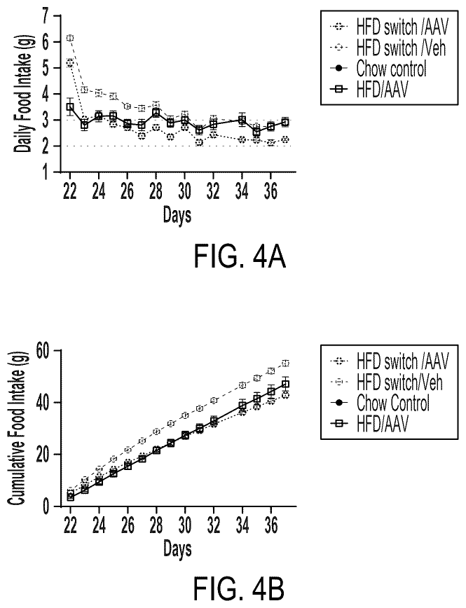

After the dietary transition at week 3, food intake patterns changed in the groups switching to high-fat diet. FIGS. 4A-4B show food intake measured in grams during weeks 3-5. The chow-HFD/vehicle and chow-HFD/AAV groups both show a decrease in daily food intake after the switch, however the chow-HFD/AAV had a larger decrease in daily intake (FIG. 4A), reflecting the appetite-suppressing effects of treatment. The HFD/AAV mice showed consistent intake. Cumulative food intake in grams (FIG. 4B) over weeks 3-5 was lower in treated groups compared to untreated.

These data demonstrated that administration of an AAV vector encoding a GLP-1 receptor agonist provides protection against diet-induced obesity and metabolic dysfunction. Pretreatment while on a normal chow diet confers protection against subsequent high-fat diet challenge, preventing both weight gain and glucose dysregulation that would otherwise occur upon dietary transition. These results support the therapeutic and prophylactic potential of AAV-mediated GLP-1 receptor agonist delivery for the prevention and treatment of obesity and related metabolic disorders.

### Example 3. Clinically Feasible, Targeted Pancreatic Delivery and Safety in a Porcine Large Animal Model

Targeted pancreatic delivery represents the preferred clinical route of administration (ROA) for delivery of GLP-1 gene therapy, as it is expected to reduce systemic biodistribution, minimize AAV exposure, and enable lower vector doses. To achieve this approach, an endoscopic ultrasound (EUS)-guided intrapancreatic infusion device and procedure for direct delivery to the pancreatic splenic lobe was developed. The device features a custom 27 G echogenic injection needle for improved visualization, a conical tip with a lateral side-port to reduce tissue trauma and backflow, and a pressure-regulated infusion system enabling two consecutive 1-mL injections at distinct sites within the pancreas splenic lobe. The procedure duration is approximately 20 minutes under real-time imaging.

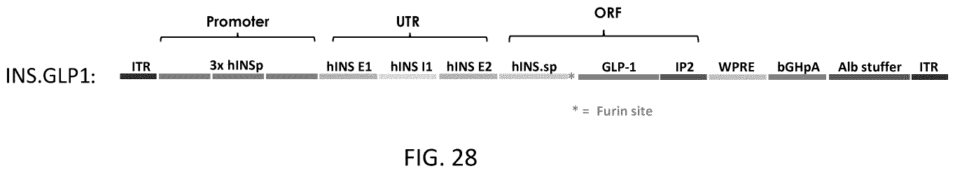

The Yucatan minipig was selected as the large animal safety model due to its anatomic similarity to humans, which enables procedural similarity, as well as its comparable pancreatic architecture and established use in endoscopic and gene therapy research. In a 6-week biodistribution and toxicology study, (Eight ~28-month-old male and female minipigs weighing 34-51 kg) received either vehicle (n=2; two females) or AAV9.INS.GLP1 (1.35E13 total vg [low] (n=3; two males, one female) and 3-fold higher: 4.04E13 total vg [high] (n=3; two males, one female), via EUS-guided intrapancreatic infusion. The viral vector genome of AAV9.INS.GLP1 is shown in FIG. 28. Endpoints included biodistribution, transgene expression, immune and clinical chemistry profiles, and histopathology.

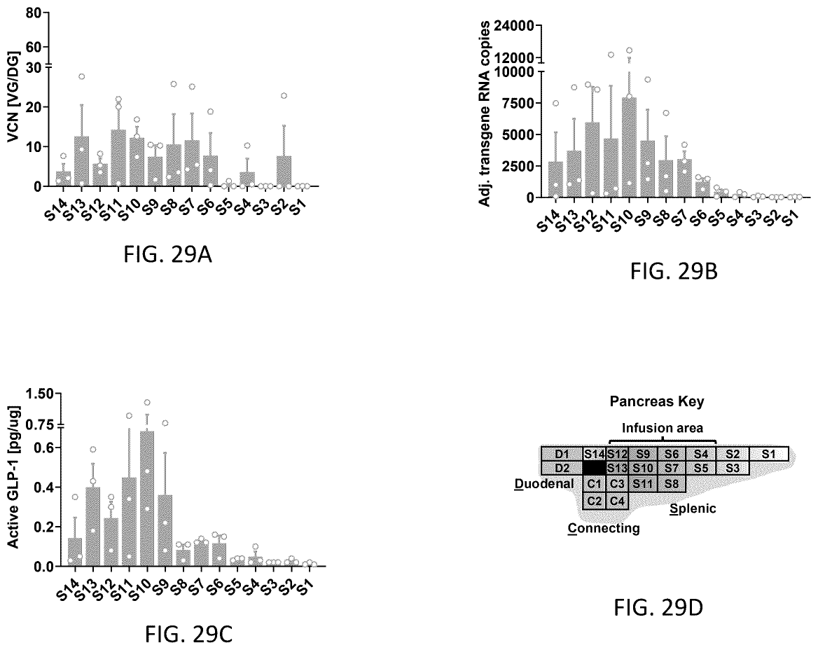

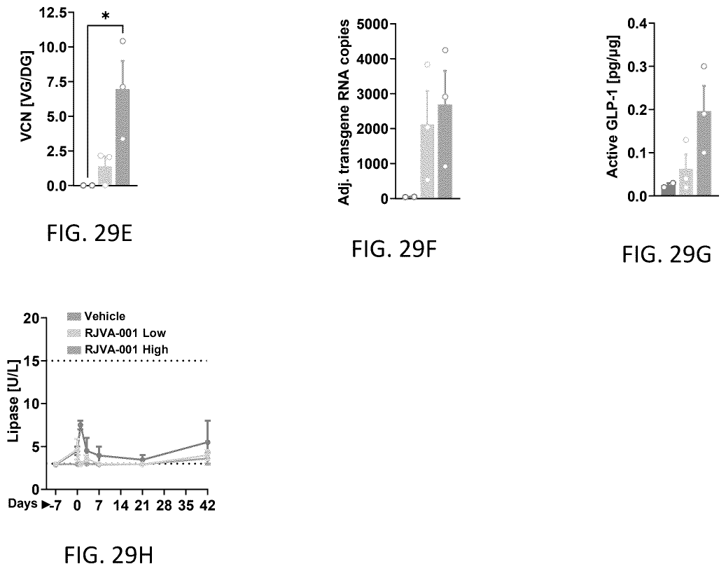

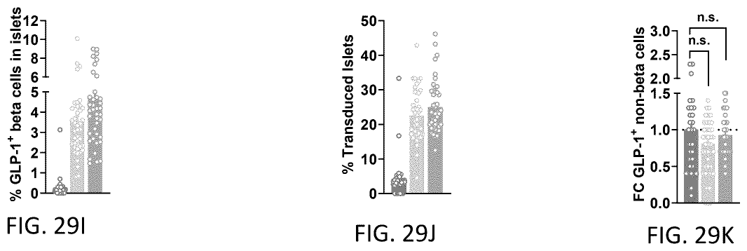

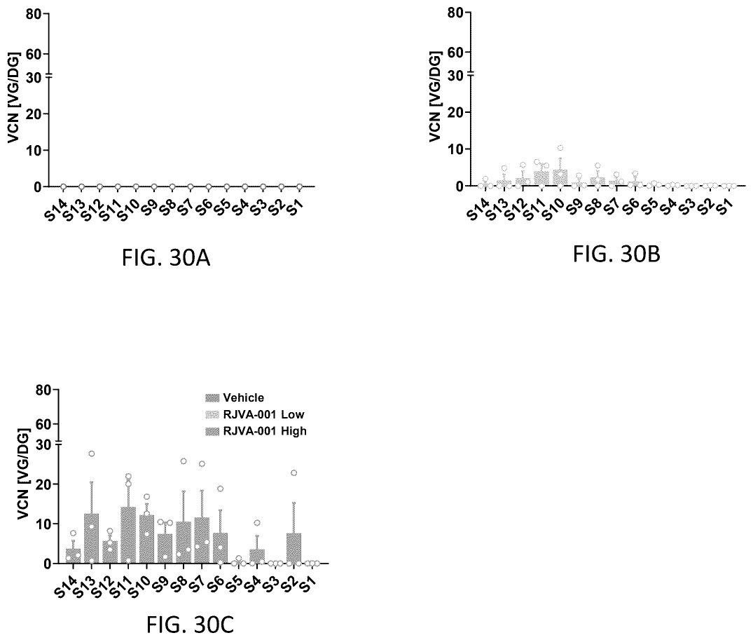

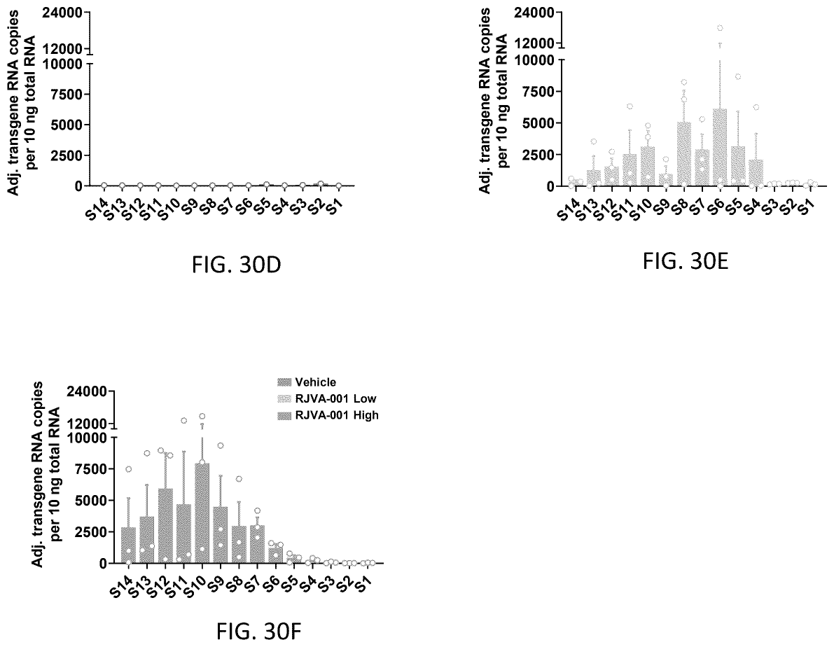

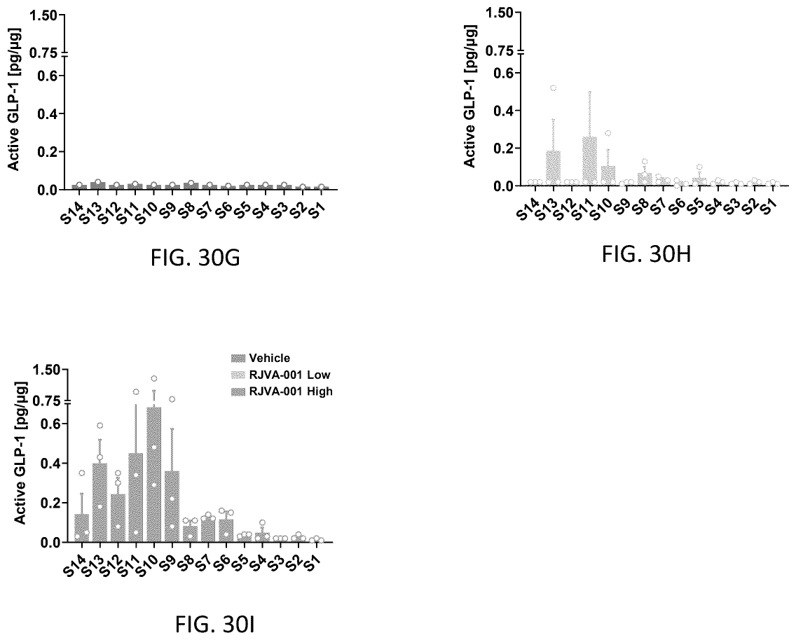

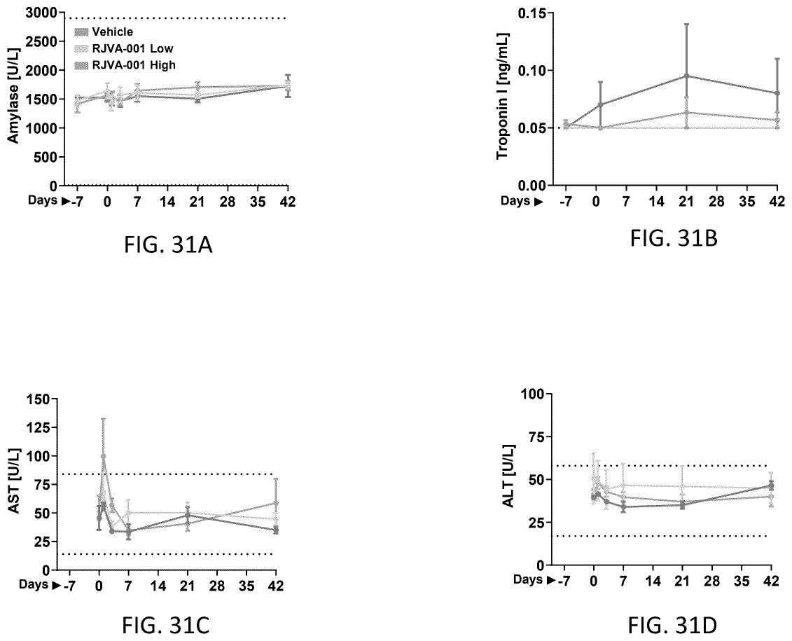

Quantitative ddPCR, RT-ddPCR, and MSD analysis of terminal biopsies demonstrated targeted transduction and expression within the islet-enriched pancreas splenic lobe (FIGS. 29A-29D and FIG. 30A-30I). Group mean pancreatic VCN, transgene RNA copies, and active GLP-1 levels each showed dose-responsive increases relative to vehicle controls (FIGS. 29E-29G). No signs of pancreatitis were observed; serum lipase and amylase remained within the normal ranges (FIG. 29H and FIG. 31A). Similarly, troponin I levels were stable, indicating absence of cardiotoxicity, and transient, mild elevations in aspartate aminotransferase resolved by day 3 with alanine transaminase remaining normal throughout (FIGS. 31B-31D).

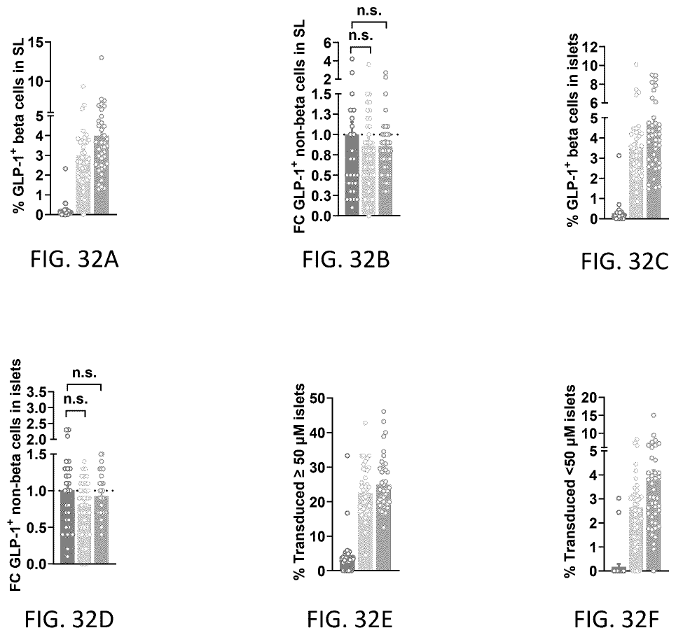

Dual immunofluorescence for insulin and GLP-1 confirmed beta cell-restricted expression within the pancreas. Vehicle-recipient animals displayed GLP-1 immunoreactivity only in alpha cells, as expected. In contrast, AAV9.INS.GLP1-treated animals exhibited elevated GLP-1 expression co-localized with insulin in islets, with no signal above background in exocrine tissue (data not shown). Quantitative image analysis revealed a ~5% mean increase in dual insulin+/GLP-1+ cells across the pancreas splenic lobe and transgene expression in ~25% of all islets ≥50 microns in area (FIGS. 29I-29J and FIGS. 32A-32F). Insulin-/GLP-1+ immunostaining remained at background levels, confirming beta cell-specificity (FIG. 29K).

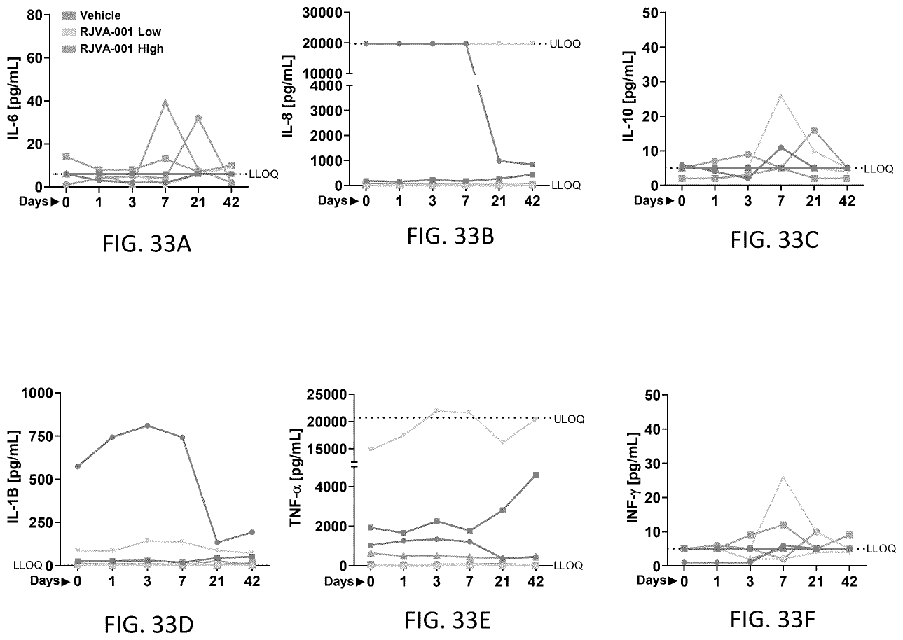

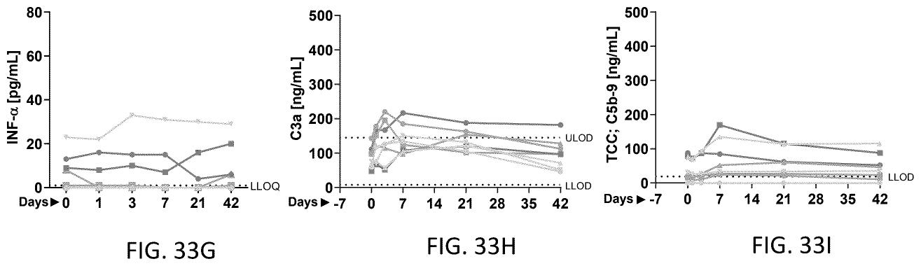

Innate immune responses were assessed via cytokine and complement profiling pre-dose and throughout the study. Baseline variability was observed across groups, including vehicle animals, and elevated pre-dose cytokine levels in some animals remained consistently higher throughout the study, irrespective of treatment. Transient cytokine elevations, when observed, generally resolved by Day 7 or Day 21 and were not associated with clinical signs or evidence of sustained immune response (FIGS. 33A-33G). Complement activation was measured by assessing both the C3a marker for early pathway activation and the terminal complement complex (TCC; C5b-9). A modest C3a elevation was transient and self-limited: observed by Day 7, including in vehicle animals, and progressively declined toward baseline (FIGS. 33H-33I). Treatment did not activate the terminal complement complex based on C5b-9 measurements, which remained at baseline levels. Together, these results indicate that a mild, non-propagated complement response occurred in some cases within days of the procedure and subsequently resolved.

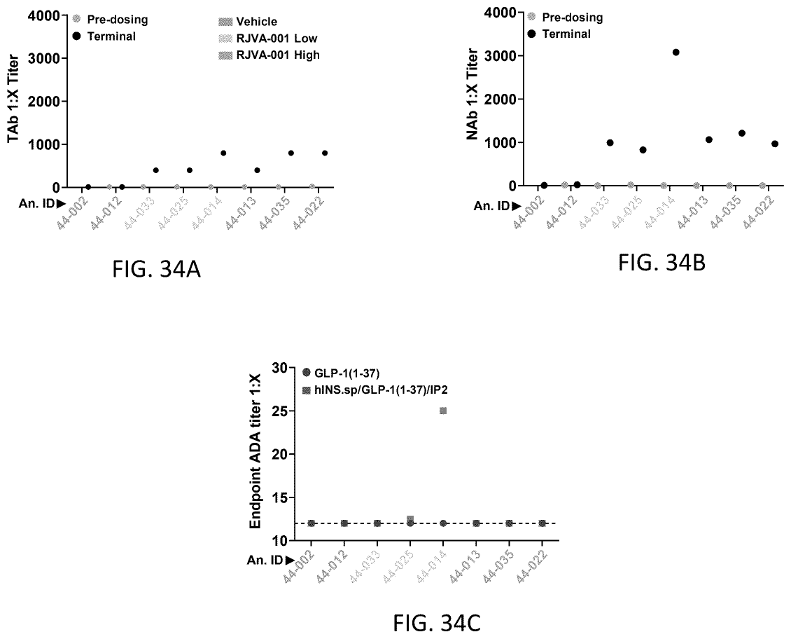

Humoral and cellular immunity were also evaluated pre-dosing and at the 6-week terminal timepoint. As expected, AAV9 total (TAb) and neutralizing antibodies (NAb) titers rose in treated groups (FIGS. 34A-34B). Table 1 shows a summary of ELISpot testing of peripheral blood mononuclear cells (PBMC) collected pre-dosing (Pre) and terminally (Post) for cellular response to AAV9 capsid peptides divided into three pools (Pool 1+2, Pool 3+4, Pool 5+6). Minus signs indicate negative response signal (dark-lined box if lost) and plus signs indicate positive response signal (dark-lined box if acquired). The ELISpot assays for AAV9-specific T-cell responses revealed variable reactivity and no correlation was observed between treatment and cell-mediated immunity to AAV9 (Table 1). Humoral immune response was also tested against the GLP-1₍₁₋₃₇₎ fragment (homologous to human and pig) and the full-length transgene protein (including flanking hINS.sp and IP2 regions homologous to human but non-homologous to pig). Two treated animals generated modest levels of antibodies (Abs) against the full-length protein, but no animals generated Abs against the GLP-1₍₁₋₃₇₎ domain. These results show a favorable immunogenicity profile for AAV9.INS.GLP1, demonstrating that the core protein did not elicit an immune response and that any immune response against the flanking portions due to incomplete sequence homology in pigs did not result in a cascade effect that could lead to Ab generation against the core GLP-1₍₁₋₃₇₎ region (FIG. 34C).

TABLE 1
ELISpot testing of peripheral blood mononuclear cells (PBMC)

| | | Pool 1 + 2 | | Pool 3 + 4 | | Pool 5 + 6 | |
|---|---|---|---|---|---|---|---|
| Treatment | ID | Pre | Post | Pre | Post | Pre | Post |
| Vehicle | 44-002 | − | − | − | − | − | − |
| | 44-012 | − | − | + | + | + | 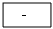 |
| Low | 44-033 | − | − | + | + | + | + |
| | 44-025 | − | 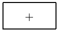 | + | + | + | + |
| | 44-014 | − | − | + | + | − | − |
| High | 44-013 | − | − | + | + | + | 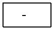 |
| | 44-035 | − | − | + | + | + | 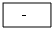 |
| | 44-022 | − | − | + | + | − | 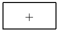 |

Dark-lined boxes with dashes indicate lost reactivity from pre to post dose.
Dark-line boxes with pluses indicate gained reactivity from pre to post dose.

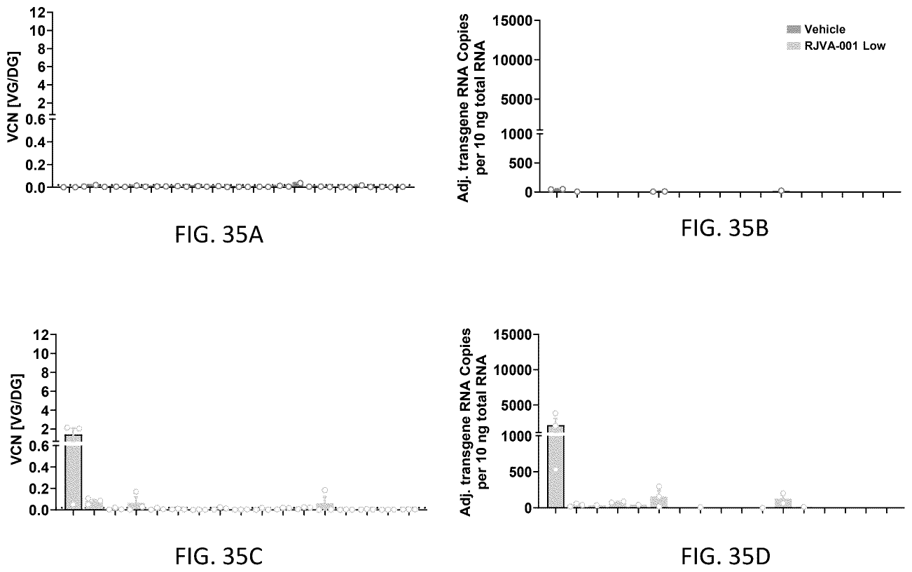

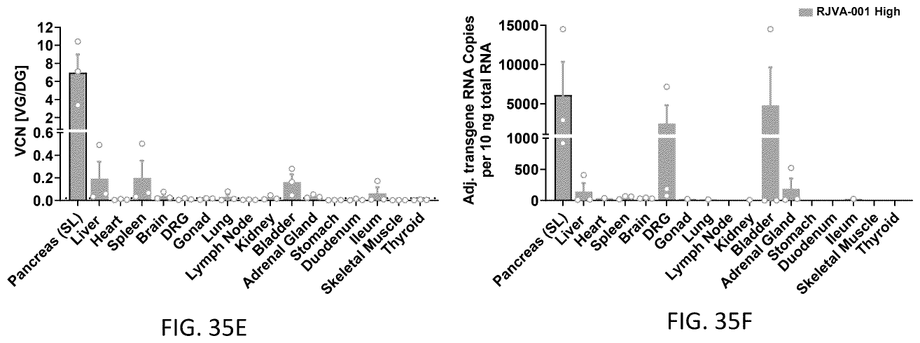

Biodistribution and subsequent transgene RNA expression analyses in VCN+ samples across a panel of off-target tissues showed highly enriched vector genome and RNA levels in the pancreas splenic lobe, with minimal or background-level signals elsewhere (FIGS. 35A-35F). Mean pancreas splenic lobe vector and transgene RNA copy numbers were 36-fold and 42-fold higher than in the liver, respectively, demonstrating relative confinement of expression to the targeted region. Histopathological assessment by a board-certified pathologist of 18 off-target tissues supported these findings, revealing only mild, predominantly incidental findings, with no consistent evidence of systemic toxicity. Any findings were low-grade, focal, and either consistent with typical background lesions well-characterized in Yucatan minipigs or artifacts related to euthanasia or necropsy procedures.

Within the pancreas, one animal receiving the high dose (4.04E13 vg) had a focal region of potential intraparenchymal necrosis mixed with postmortem changes. There was occasional perivascular/periductal mononuclear or mixed inflammatory cell infiltrates observed in the peripancreatic tissue or interlobular septa of the pancreas, with frequency and presence of cellular infiltrates being slightly increased from minimal to moderate in animals receiving AAV. Otherwise, there was no evidence of systemic dissemination or generalized pancreatic injury. Acinar and islet architecture remained intact, consistent with non-degenerative, localized tissue response to the infusion procedure.

Collectively, these results demonstrate that EUS-guided pancreatic infusion of AAV9.INS.GLP1 achieves efficient, localized, beta cell-specific gene transfer with minimal systemic exposure and no evidence of off-target toxicity. This clinically feasible, endoscopic delivery route supports translation of AAV9.INS.GLP1 as a targeted gene therapy for T2D.

### Example 4. Toxicology Study for AAV9.INS.GLP1 with Delivery Procedure

A toxicology study was designed under good laboratory practice (GLP) to evaluate the delivery, expression, and safety of treatment with AAV9.INS.GLP1. AAV9.INS.GLP1 was tested in Yucatan pigs at three dose levels to capture a range of doses relevant to efficacy, safety, and an expected minimal efficacious dose in humans.

### Study Design

Following AAV9 total antibody (Tab) screening and selection of Yucatan pigs with acceptably low titer (≤1:25 dilution), 12 subjects aged ~18 months weighing 33.0-48.4 kg underwent endoscopic ultrasound (EUS)-guided pancreatic infusion with either vehicle or AAV9.INS.GLP1 at a low, mid, or high dose (matching the dosing of the toxicology study from Example 3) for a 6 w study (Table 2). All pigs received prophylactic immunosuppression (Tacrolimus [0.05 mg/kg] and MMF [500 mg] 2× daily; days-3 to 7) to avoid potential immune response to AAV9 capsid and to resemble expected clinical settings in future FIH studies. Each delivery procedure used a custom delivery device and included two infusions to the pancreas splenic lobe; I: 0.2 mL saline (residual from device priming)+1 mL AAV and II: 1 mL AAV-totaling 2 mL AAV+0.2 mL saline per pancreas.

TABLE 2
Treatment Groups

| | | | Verified | | |
|---|---|---|---|---|---|
| | | | Total | | |
| | | | Dose | | Duration |
| Group | Treatment | Description | [vg] | N | [weeks] |
| 1 | Vehicle | Vehicle control | N/A | 3 | 6 |
| 2 | AAV9.INS.GLP1 | Low dose | 1.31E13 | 3 | 6 |
| 3 | AAV9.INS.GLP1 | Mid dose | 3.96E13 | 3 | 6 |
| 4 | AAV9.INS.GLP1 | High dose | 1.50E14 | 3 | 6 |

### Methods

Endpoints focused on safety, biodistribution, expression, and specificity and included:
Clinical pathology: hematology, serum chemistry, lipase/amylase enzymes (pancreatitis biomarkers), Troponin I (TrnI; cardiotoxicity biomarker), Neurofilament Light chain (Nf-L; neurotoxicity biomarker), ALT/AST (hepatotoxicity biomarker), FPI and Fasting C-peptide (FCP) (hypoinsulinemia markers), Blood glucose (BG; hypoglycemia marker).
Histopathological assessment in on-target (pancreas; ONT) and off-target (OFT) tissues.
Immunogenicity: Pre-dose and terminal AAV9 TAb and ELISpot and anti-drug antibody (ADA) against transgenic GLP-1 protein.
Viral shedding and clearance: Blood, urine, feces, and saliva.
Biodistribution and transgene (TG) RNA expression in ONT and OFT tissues. GLP-1 protein levels in pancreas and plasma. Promoter specificity in islets by INS/GLP-1 IF

### Observations/Results

All animals underwent successful EUS infusion, with the endoscopist confirming correct needle placement via real-time ultrasound imaging. Throughout the study, all animals underwent regular monitoring for abnormal behavior that could indicate discomfort, and no issues were observed.

### Clinical Pathology

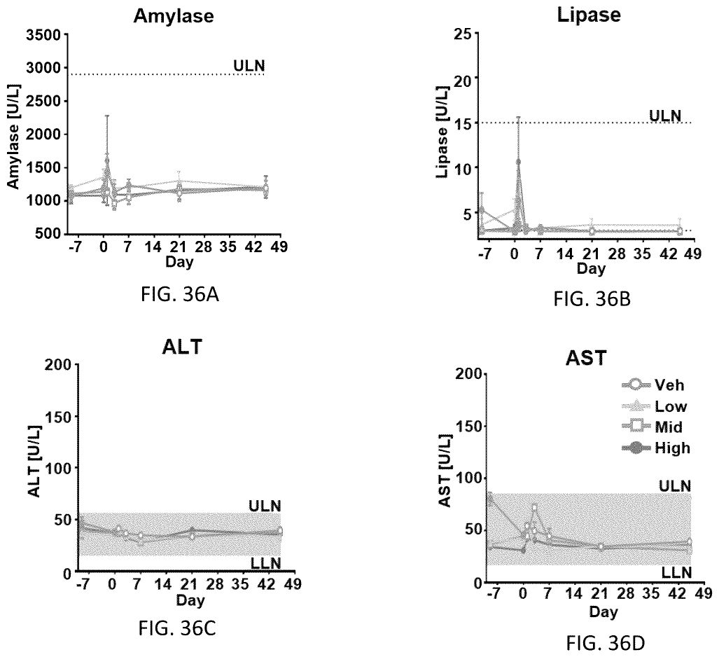

As shown in FIGS. 36A-36D, hematology and serum chemistry results were within normal limits. No pancreatic toxicity was observed as serum amylase and lipase levels remained within normal limits except for one high dose animal (ID 44-010) that demonstrated elevated values just above the upper limit of normal (ULN) on Day 1 that resolved by Day 7 (FIGS. 36A-36B). Transient elevations at this timepoint are considered procedure-related rather than drug-induced. Transient spikes in lipase that remain below 3×ULN are not considered to be a safety risk. No hepatoxicity was observed as serum ALT and AST remained within normal range (FIGS. 36C-36D). Transient, mild elevation in AST on Day 1 that resolved by Day 7 is likely attributable to sedation activities during the procedure.

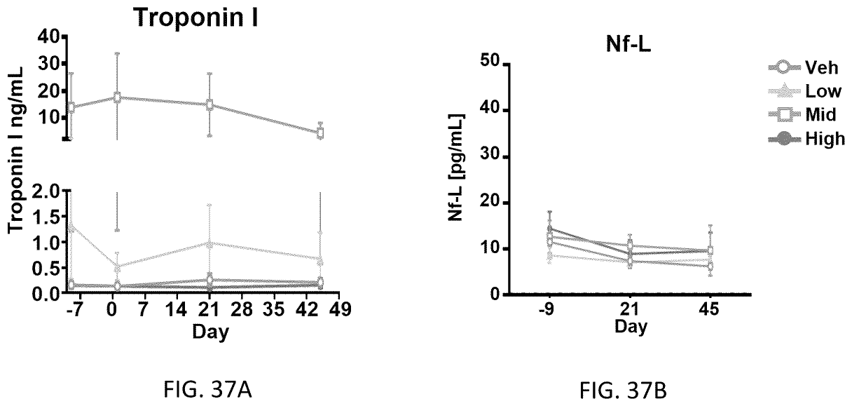

No evidence of cardiotoxicity was observed. Troponin I levels were not impacted by AAV9.INS.GLP1 treatment, with each animal remaining at its baseline level, including one mid-dose animal (ID 44-019) with a high baseline (FIG. 37A). Likewise, no evidence of neurotoxicity in response to treatment was observed using NfL as a biomarker (FIG. 37B).

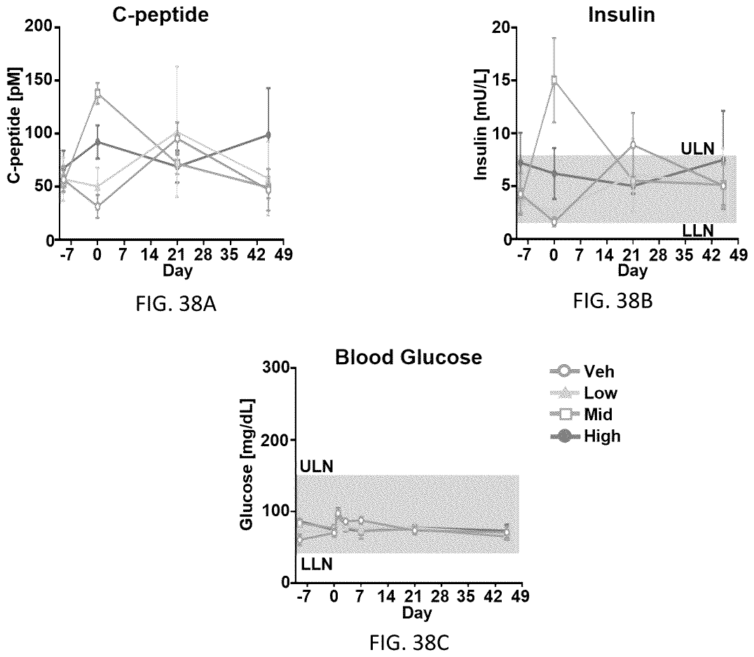

No evidence of hypoinsulinemia or hypoglycemia was observed, as plasma insulin, plasm C-peptide, and blood glucose levels did not appear to be impacted by treatment and maintained vehicle-like levels throughout the study (FIGS. 38A-38C).

These results strongly support the safety of the AAV9.INS.GLP1 procedure and treatment.

### Immunogenicity Assessment

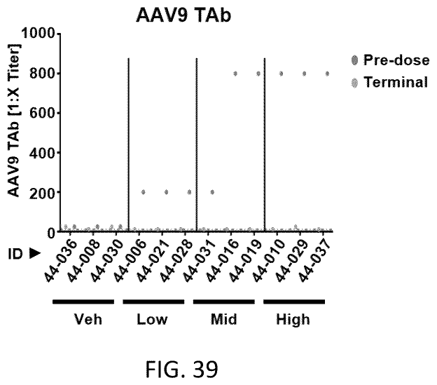

Humoral immune response to AAV9 capsid was assessed by applying a TAb assay to pre-dose and terminal blood samples, which showed an expected increase in AAV9 TAb titer in the treated animals most pronounced in the high dose group (FIG. 39). A cellular immune response against the AAV9 capsid was also assessed via an ELISpot assay consisting of three AAV9 peptide pools, which was applied to peripheral blood mononuclear cells (PBMCs) collected pre-dose and at termination (Table 3).

TABLE 3
AAV9 ELISpot Assay Results

| | | Pool 1 + 2 | | Pool 3 + 4 | | Pool 5 + 6 | |
|---|---|---|---|---|---|---|---|
| Treatment | ID | Pre | Post | Pre | Post | Pre | Post |
| Vehicle | 44-036 | − | − | − | − | − | − |
| | 44-008 | − | − | + | 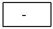 | + | 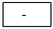 |
| | 44-030 | + | 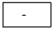 | + | + | + | 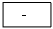 |
| AAV9.INS.GLP1 1.74E13 vg | 44-006 | − | − | − | 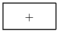 | − | − |
| | 44-021 | − | − | − | − | + | 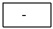 |
| | 44-028 | − | − | + | + | − | 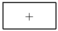 |
| AAV9.INS.GLP1 5.21E13 vg | 44-031 | + | 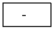 | + | + | + | 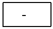 |
| | 44-016 | − | − | + | 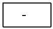 | + | 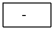 |
| | 44-019 | + | 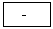 | + | + | + | + |
| AAV9.INS.GLP1 1.56E14 vg | 44-010 | − | − | + | + | − | 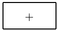 |
| | 44-029 | + | 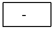 | + | + | + | + |
| | 44-037 | − | − | + | 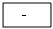 | + | + |

Dark-lined boxes with dashes indicate lost reactivity from pre to post dose.
Dark-line boxes with pluses indicate gained reactivity from pre to post dose.

All but one animal showed pre-existing reactivity to at least one peptide pool and while reactivity was gained for some animals for some peptide pools at study end, reactivity was lost for others. This heterogeneity could be attributed to inherent limitations of T cell immune monitoring (e.g., low precursor frequencies, variable functional responsiveness) and limited systemic antigen exposure. Overall, non-dose-responsive, cell-mediated immune response appeared to be modestly activated in a subset of animals.

Humoral immune response against the transgene protein in pre-dose and terminal samples was assessed via two transgene protein ADA assays-one developed against GLP-1₍₁₋₃₇₎ produced in HEK293 cells and another against a synthetically produced full-length transgene protein consisting of the human insulin secretion peptide (hINS.sp), GLP-1 (1-37) and IP2. No immune response was detected for either ADA assay in any treated animal. One vehicle showed low positivity (1:12.5 titer) for GLP-1 (1-37) in its terminal sample. Taken together, these results imply a favorable expected outcome in humans, in which the hINS.sp, GLP-1 (1-37), and IP2 domains are 100% homologous, whereas in pigs, hINS.sp and IP2 are not.

### Pancreatic Transgene Biodistribution, Expression, and Pancreatic Specificity

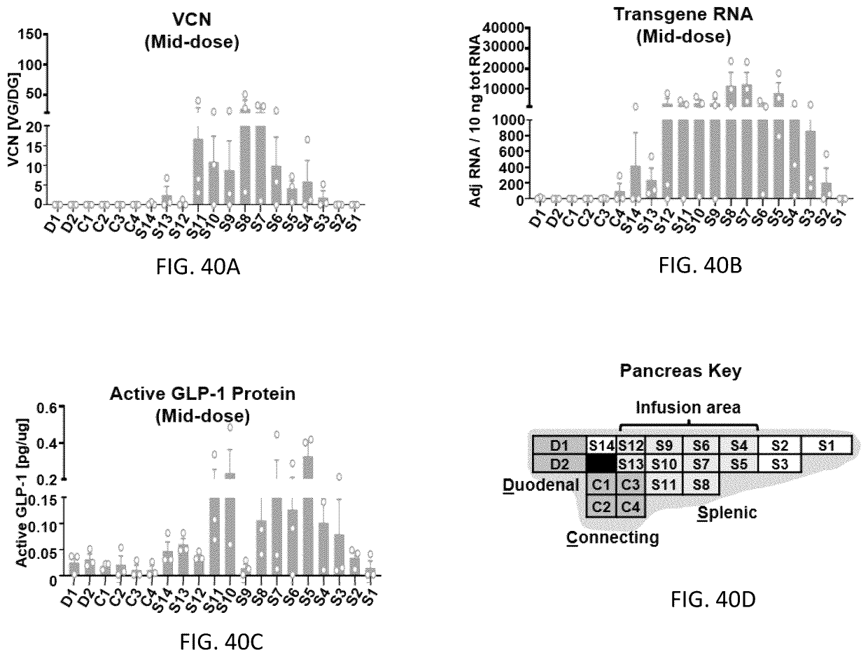

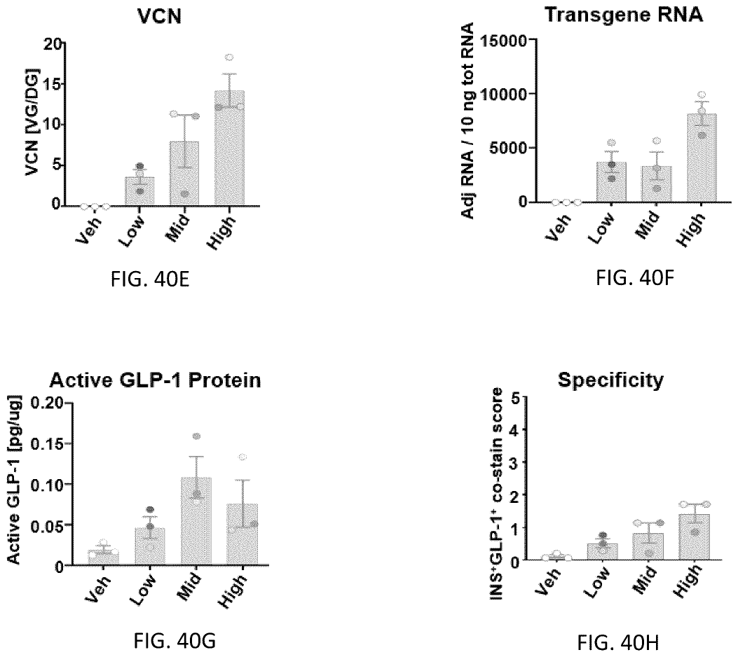

Biopsies were collected from 20 different sections across the pancreas and subjected to DNA/RNA co-isolation and protein extraction to assess biodistribution, TG RNA expression, and active GLP-1 levels. ddPCR, RT-ddPCR, and MSD detected VCN, RNA, and active GLP-1 protein levels, respectively, that trended with each other's distribution across the targeted splenic lobe (FIGS. 40A-40D). When averaging values across each group, a dose-responsive increase was observed (FIG. 40E), although the low and mid doses showed similar TG RNA copies (FIG. 40F), which could be attributable to variable RNA quality (a well-documented occurrence in pancreas tissue due to high enzymatic activity) or limited group (n) size. Active GLP-1 levels appeared to plateau at the mid-dose despite lack of VCN or RNA plateauing (FIG. 40G), which could also be attributed to variability and small group size-nonetheless reaching ~4-fold over vehicle levels. Dual insulin/GLP-1 immunofluorescence analysis of pancreas lobe biopsies showed dose-responsive co-positivity for insulin and GLP-1 expression in up to ~25% of endocrine cells (FIG. 40H). Together, these results demonstrate successful targeted AAV delivery and robust ONT transgene expression.

Transgene Expression Specificity within the Pancreas

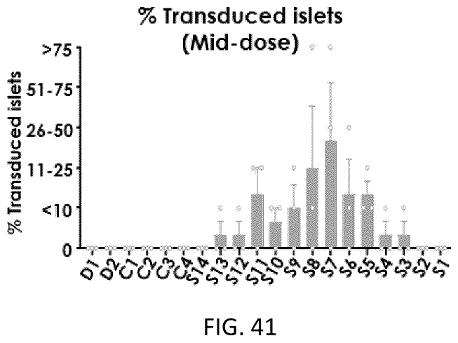

Pancreas biopsies collected from each pancreas section were subjected to dual INS/GLP-1 IF to assess the specificity of transgene expression driven by the hINSp. Vehicle animals displayed beta cells (insulin-positive) and alpha cells (endogenous GLP-1-producing) within well-defined islets and scattered clusters (data not shown). Treated animals at all dose levels displayed dual-positive beta cells, indicating co-expression of insulin and transgenic GLP-1 in the targeted cell type (data not shown). FIG. 41 shows the percentage of transduced islets (dual insulin and GLP-1 positive islets) in animals given the mid dose as determined by pathologist scoring. This pathologist scoring of co-staining identified a distribution pattern similar to that of VCN, transgene RNA, and GLP-1 protein that led to a substantial percentage of effectively transduced islets across the pancreas (FIGS. 40A-40D and FIG. 41). Results were confirmed via analysis by a trained AI model that also revealed a dose-responsive increase in the percentage of transduced beta cells that reached ~4% (data not shown). Importantly, no GLP-1 signal was observed within the exocrine space, which is considered OFT within the ONT organ. These results demonstrate the beta cell specificity of transgene expression and indicate that while a modest percentage of beta cells in each islet expressed GLP-1, a substantial percentage of islets was functionally transduced.

### Transgene Biodistribution and Expression in Off-Target Tissues

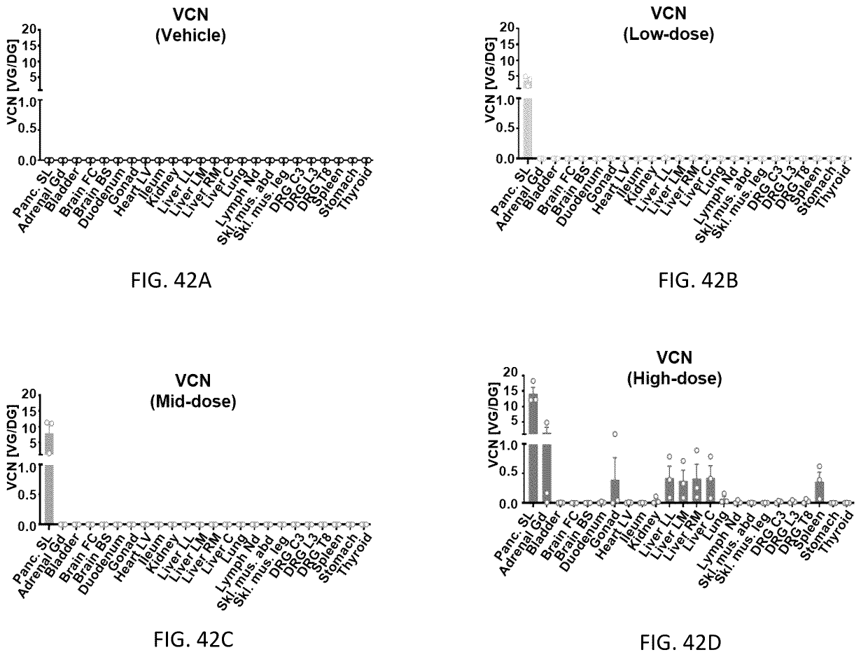

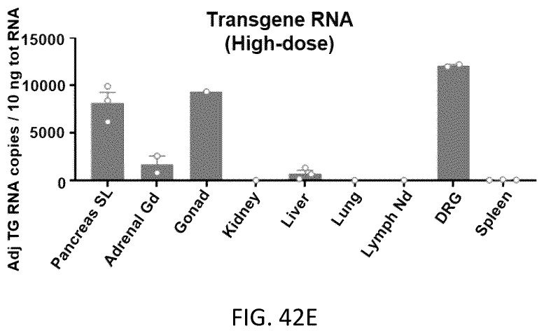

From 23 OFT tissues, DNA/RNA was extracted and subjected to biodistribution analysis. VCNs were below limit of quantitation (BLOQ) in all OFT tissues in the low and mid-dose groups (FIGS. 42A-42C). In the high-dose group, individual samples from just eight tissue types showed quantifiable VCNs, which were at least 10-fold lower than in the pancreas SL (FIG. 42D). Individuals in the high dose group were subjected to TG RNA expression analysis via RT-ddPCR (FIG. 42E). Of these samples, kidney, lung, lymph node, and spleen showed undetectable or negligible TG RNA copies. Adrenal gland and liver showed low TG RNA copies that were 5-fold and 11-fold below pancreas SL, respectively. A single gonad sample and two DRG samples showed TG RNA copies similar to pancreas SL RNA levels (FIG. 42E). Despite the noted expression in the DRG, no neurotoxicity was observed via NfL nor inflammation via histopathological assessment (documented below). Development and Reproductive Toxicology (DART) studies are also generally not needed for AAV9 vectors. Although biodistribution studies indicate limited vector distribution and transgene expression to the gonads.

### Viral Shedding and Pharmacokinetics

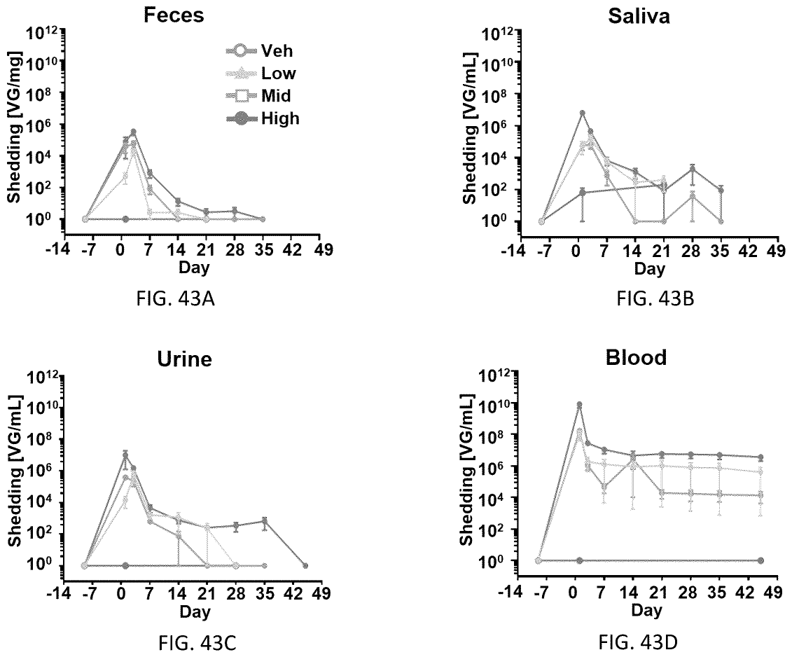

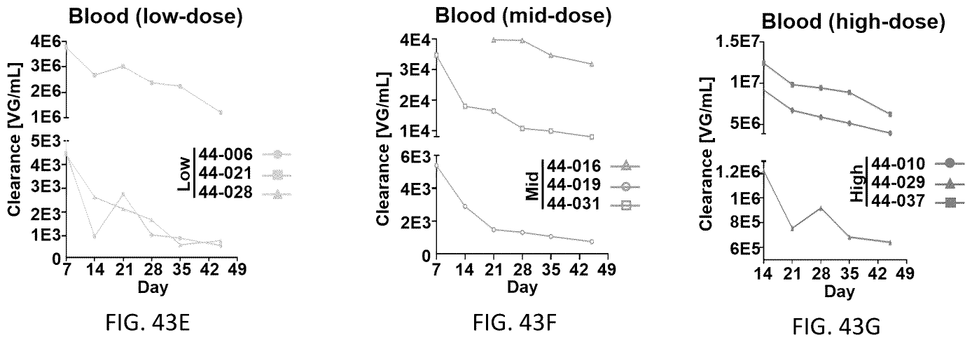

Blood, saliva, urine, and feces samples were collected pre- and post-dose at multiple timepoints to assess viral shedding and clearance via a ddPCR assay targeting the WPRE sequence within the viral genome (FIGS. 43A-43G). In all four matrixes, a dose-responsive peak in VG was observed on Day 1, as expected. This peak was followed by typical biphasic clearance in the form of an initial rapid decay over the first 7-14 days characteristic of initial clearance from circulation, and then a continued gradual decline (FIGS. 43A-43D). In feces, no signal was observed by Day 35 (FIG. 43A). In saliva, signal was at background vehicle levels by Day 35 (FIG. 43B). In urine, low and mid-dose animals reached below limit of detection (BLOD) levels by Days 14-28, while high dose animals reached BLOD at Day 45 (final timepoint) (FIG. 43C). In blood, VG copies continued to decline yet remained abundant on Day 45 (study end) (FIGS. 43D-43G), suggesting gradual clearance of VGs from transduced tissues. Collectively, these results support minimal risk of environmental exposure or horizontal transmission.

Overall, the results indicate highly selective biodistribution and TG RNA expression in the pancreas SL vs a comprehensive range of representative OFT tissue types.

### Histopathological Assessment

Pancreas and OFT tissue samples were subjected to H&E staining and histopathological analysis (data not shown). In the pancreas, only mild inflammation and necrosis were observed in both vehicle and treated animals near the anticipated infusion site. There was no evidence of dose-related exacerbation of inflammation, fibrosis, necrosis, or other degenerative changes. Any observed rare, mild fibrosis and islet atrophy were without consistent pattern or severity across groups. In OFT tissues, including liver, heart, kidney, brain, DRG, adrenal gland, and gonad, no histological abnormalities attributable to the test article were observed. In the CNS and DRG, no neuronal degeneration, inflammation, or satellite cell proliferation were observed in any treated animals. These results strongly support the safety of AAV9.INS.GLP1 treatment.

### CONCLUSION

All animals underwent successful EUS infusion. No evidence of pancreatitis, hepatoxicity, cardiotoxicity, neurotoxicity, hypoinsulinemia, or hypoglycemia was observed. An AAV9 TAb dose-responsive titer increase was observed, as expected. A modestly activated AAV9 cellular response was observed in a subset of animals (ELISpot). There was no generation of ADA against the transgene protein. Dose-responsive delivery and transgene expression in the targeted pancreas splenic lobe was achieved. Beta cell-specific expression within the pancreas splenic lobe was confirmed via immunofluorescence analysis and a substantial percentage of functionally transduced islets were observed. There was minimal off-target (OFT) biodistribution. No VCNs were detected at low or mid-dose, and only low-level VCNs were detected in several tissues at high dose (still >10× lower than in pancreas). There was limited OFT transgene RNA expression in OFT tissues. There was minimal risk of environmental exposure or horizontal transmission as confirmed via viral shedding and clearance analysis. Although biodistribution studies indicate limited vector distribution and transgene expression to the gonads, Zhao et al. supports the absence of AAV-based germline transmission since AAV9 was not found to transduce sperm or oocytes. There was no dose-related exacerbation of inflammation, fibrosis, or necrosis in the pancreas. No abnormalities in OFT tissues were observed. No neuronal degeneration, inflammation, or satellite cell proliferation in the CNS and DRG was observed.

### Example 5. Endoscopic Delivery to the Pancreas

Aspects of the present technology include a method of conducting an infusion procedure, including, for example, using example devices of the present disclosure. Example methods include positioning an endoscope within the gastrointestinal tract, identifying anatomical landmarks via ultrasound imaging, advancing a catheter through the working channel of the endoscope, and delivering a therapeutic agent into the pancreas while avoiding critical vascular and ductal structures.

In an example method, the patient can be positioned in a supine or left lateral decubitus position on the procedure table. A bite block can be inserted between the patient's teeth to protect the endoscope and maintain airway patency. It can be ensured that there can be no air within the endoscopic ultrasound balloon by filling and suctioning the balloon multiple times to minimize ultrasound artifacts. The overtube can be lubricated to reduce friction during endoscopic advancement.

The endoscope can be introduced into the patient's mouth and advanced through the oropharynx into the esophagus while maintaining visualization. The airway can be avoided by identifying and bypassing the laryngeal structures. The endoscope can be then advanced into the stomach, and excess air can be suctioned from the gastric space to optimize ultrasound imaging. Ultrasound mode can be activated, and the transducer can be positioned against the gastric wall to begin visualization of anatomical landmarks.

The aorta can be located as a primary landmark by identifying a pulsatile, thick-walled tubular structure using Doppler imaging to confirm blood flow. The ultrasound probe can be positioned inferior to the gastroesophageal junction and adjusted by rotating the endoscope and manipulating the control wheels. If the liver can be visualized, the endoscope can be slightly withdrawn to avoid mistaking the intrahepatic inferior vena cava for the aorta. The aorta can be followed distally until the celiac trunk can be identified as a branch point from the aorta. The splenic artery can be tracked as it branches from the celiac trunk and runs adjacent to the splenic vein along the superior border of the pancreas. The pancreas can be identified as a diffuse, hyperechoic, heterogeneous structure surrounding the splenic vasculature. Confirmation of pancreatic identity can be achieved by locating small intrapancreatic ducts, which appear as non-vascular, tubular structures with no detectable flow on Doppler imaging.

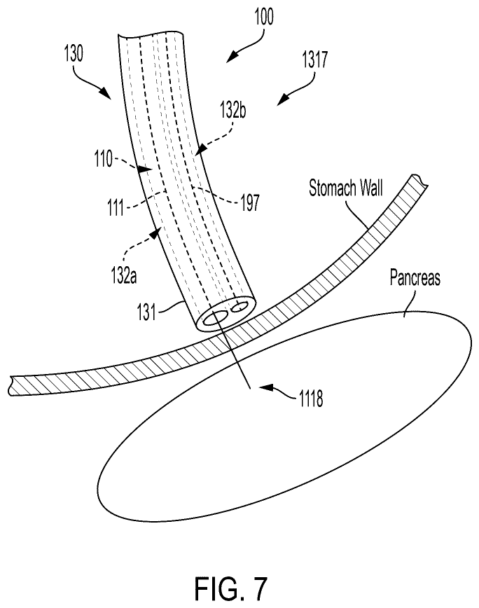

Once the pancreas can be identified, a catheter can be advanced through the working channel of the endoscope and positioned with a clear path to the target region of the pancreas. FIG. 7 illustrates a non-limiting example. As shown, the infusion system 100 includes an endoscope 130 positioned in the stomach against the stomach wall. A lumen 111 disposed in a working channel of the endoscope extends against the stomach wall and a needle at the end of the lumen is extended to puncture through the stomach wall and into the pancreas. In this configuration, the endoscope (and therefore the needle) may be directed orthogonal to the length of the pancreas, and may be positioned lose to the tail of the pancreas or body of the pancreas. Multiple infusions, involving multiple punctures, may be performed at different points in the pancreas.

Puncture of adjacent arteries, veins, and ducts can be avoided to minimize the risk of bleeding or pancreatitis, as well as to optimize delivery of the therapeutic dose to the intended location. The catheter sheath length can be adjusted to ensure approximately 5 mm of sheath can be visible in the ultrasound field, confirming the projected needle trajectory. Anatomical positioning can be verified by re-identifying the splenic artery and vein using rotational adjustments of the endoscope.

The syringe adapter can be attached to the catheter handle to facilitate controlled infusion and facilitate control of infusion location. The elevator on the endoscope can be adjusted to modify the needle's angle of approach. The needle depth adjustment mechanism can be set to allow the needle to advance into the pancreatic parenchyma while avoiding overextension beyond the target region. Upon penetration of the pancreatic capsule, a tactile resistance change may be observed, indicating successful entry. The lock ring can be used as a hard stop to prevent excessive needle advancement. The endoscopist signals to the pump operator that the needle can be in position, and the infusion of the therapeutic agent can be initiated by opening the device valve. In practice, the initiation can first include 2. Opening the emergency stop in the direct fluid line, then toggling the trigger safety under the infusion button against the handle to off, depressing the infusion button, letting the infusing complete, and then closing the emergency stop and returning the trigger safety back under the infusion button. The infusion process can be monitored (e.g., under EUs) to ensure that the needle remains stable and that a localized fluid collection (bleb) appears around the needle tip, confirming intraparenchymal delivery.

Following infusion, the needle can be maintained in position for at least 60 seconds to facilitate uniform agent diffusion and pressure equilibration. If a flush step can be required, the valve can be closed, the infusate syringe can be replaced with a flushing agent syringe, and the valve can be reopened to administer the flushing solution. Upon completion of the infusion or flush, the valve can be closed, and the needle can be retracted from the pancreas. The catheter can be removed from the endoscope and disposed of in a biohazardous sharps container. Prior to endoscope removal, air can be suctioned from the gastric space to minimize post-procedure discomfort. The endoscope can be withdrawn, cleaned with saline, and prepared for subsequent use.

The present method provides a standardized approach for endoscopic ultrasound-guided access to the pancreas, facilitating safe and effective delivery of therapeutic agents while mitigating procedural risks.

### Needle Size and Geometry Testing

A study was conducted to evaluate and optimize the needle geometry and diameter for minimizing pancreatic puncture forces, aiming to reduce procedural complications such as post-procedure pancreatitis. Two needle diameters (25 G and 27 G) and two needle-tip geometries (pencil-point and bevel, approximately) 12°-15° were tested. A pencil-point tip geometry was of particular interest due to its documented ability in peer-reviewed literature to reduce tissue trauma and the potential for needle occlusion compared to conventional beveled (hypodermic) needles. Moreover, the pencil-point tip geometry is also inherently safer because more traditional bevel tips are designed for cutting and coring, and examples of the present the pencil-point (e.g., conical tip) achieve, in comparison, tissue entry that includes primarily tissue displacement. As a result, certain sensitive tissue structure, such as blood vessels, are preferentially displaced during needle entry without cutting.

The study compared the peak puncture force performance of each needle geometry and diameter, focusing on identifying the configuration that minimized the force required to penetrate pancreatic tissue. Lower puncture forces are hypothesized to reduce tissue tenting, minimize unnecessary pancreatic movement, and thereby mitigate inflammatory response and post-procedure complications.

Additionally, repeated puncture tests were conducted with each needle geometry and size to simulate clinical conditions involving multiple injections (e.g., three consecutive injections through the stomach into the pancreas). This allowed assessment of potential needle tip dulling or deformation and its impact on puncture forces across multiple insertions.

Finally, the puncture forces measured for the experimental needles (particularly the optimized 27 G pencil-point needle) were compared directly against a commercially available off-the-shelf (“OTS”) 25 G (FNA) needle, previously employed in preclinical EUS pancreas studies. This comparison demonstrated that the optimized 27 G needle geometry provided equal or lower puncture forces compared to the existing larger-diameter needle, thereby illustrating a meaningful technical advantage in terms of reduced tissue disruption and enhanced procedural safety.

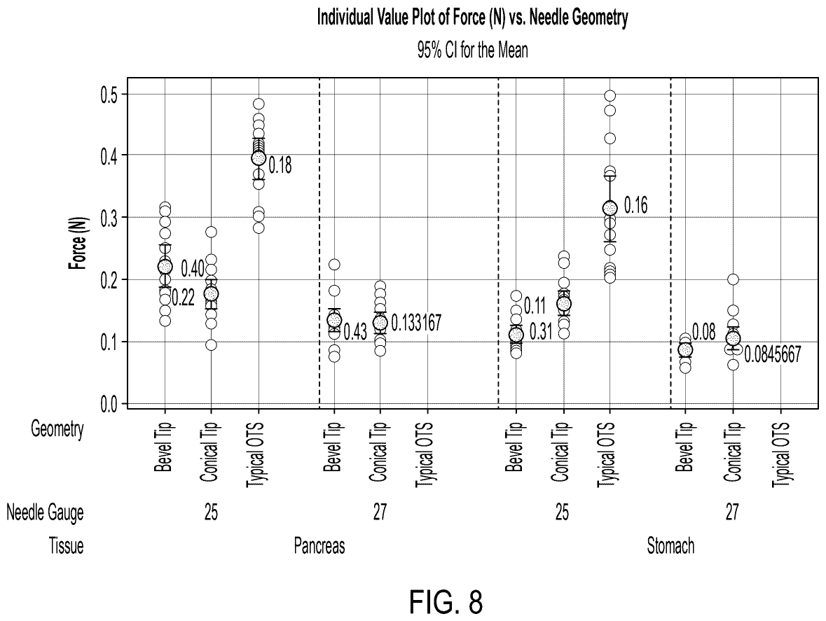

Results demonstrating these comparisons and advantages are shown in FIG. 8.

Testing was also performed to evaluate puncture forces through synthetic stomach tissue, with known physical property equivalence, consistent with procedural methodology involving alternating stomach and pancreas punctures. Needle insertion events through the stomach were interleaved with pancreas insertions, simulating actual clinical conditions and assessing cumulative needle performance across multiple punctures.

Initial stomach puncture data demonstrated that both needle diameter and tip geometry significantly influenced puncture forces, with needle diameter having the greatest overall impact. Specifically, smaller-diameter needles (27 G) consistently required lower puncture forces compared to larger-diameter needles (25 G).

Importantly, after the first stomach puncture, needle diameter became the consistently dominant factor influencing puncture force, overshadowing the initial effect of tip geometry. Additionally, puncture force trends over repeated insertions differed markedly between needle geometries. Standard bevel-tip needles exhibited progressively increasing puncture forces across repeated insertions, indicative of potential tip dulling or deformation with repeated tissue penetrations. In contrast, pencil-point needles demonstrated stable or decreasing puncture forces over multiple insertions, suggesting superior durability and sustained ease of tissue penetration.

Notably, after the second stomach puncture event, the 27 G pencil-point needle achieved equivalent or lower puncture forces compared to standard bevel-tip 25 G needles, highlighting an additional advantage of the optimized 27 G pencil-point configuration in reducing cumulative tissue trauma during repeated penetrations.

These key findings underscore the technical superiority of the optimized 27 G pencil-point needle design for multi-puncture procedures involving alternating stomach and pancreas insertions.

Puncture force measurements were also collected from pancreas tissue, using the same needles employed for stomach punctures, consistent with clinical procedures involving sequential stomach and pancreas penetrations. Across five sequential pancreas punctures, needle diameter consistently emerged as the most influential factor determining puncture force, with the smaller 27 G needles requiring notably lower forces compared to larger diameter 25 G needles.

While needle tip geometry showed less pronounced influence, notable trends emerged. Specifically, during the initial pancreas puncture, pencil-point needles demonstrated slightly higher puncture forces compared to bevel-tip needles. However, in subsequent pancreas punctures (second through fifth punctures), the pencil-point needles consistently yielded lower average puncture forces compared to standard bevel-tip needles. The observed trend in reduction in puncture forces with pencil-point needles remained highly consistent across repeated trials, indicating potential cumulative benefits of pencil-point geometry in reducing pancreatic tissue trauma over multiple insertions.

These pancreas puncture data further support the advantageous properties of the optimized 27 G pencil-point needle design, highlighting consistent reductions in puncture force and potential clinical benefits related to tissue preservation and reduced procedural complications.

The primary factor influencing puncture force was needle diameter, with smaller-diameter needles (27 G) consistently requiring lower puncture forces compared to larger-diameter needles (25 G). Needle tip geometry also impacted puncture forces: pencil-point needles initially showed slightly higher average puncture forces than standard bevel-tip needles of the same gauge. However, over multiple punctures, the pencil-point needles exhibited superior durability, maintaining more consistent (lower or stable) puncture forces, whereas standard bevel-tip needles demonstrated a progressive increase in puncture force, likely due to tip dulling or deformation.

Notably, all experimental needles tested-including both pencil-point and bevel-tip designs-exhibited lower puncture forces compared to the commercially available 25 G Beacon FNA needle, previously used as a procedural standard. While the Beacon needle demonstrated minimal dulling over repeated insertions, its consistently higher puncture forces suggest that improvements to puncture force are readily achievable by adopting the optimized 27 G needle configurations (particularly pencil-point geometries) disclosed herein. Thus, the optimized 27 G needle provides a clear technical advantage by significantly reducing puncture forces, minimizing tissue trauma, and potentially lowering procedural complications compared to existing commercial standards.

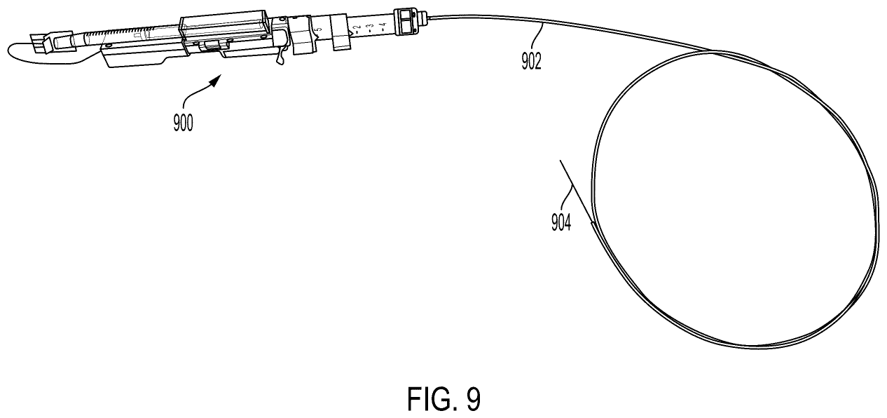

As has been described above, aspects of the present technology provide a handheld infusion system for infusing gene therapy compositions of the types described herein, as a non-limiting example. FIG. 9 illustrates an example of such an infusion system, including a handle 900, lumen 902, and needle 904. The handle, lumen, and needle may take various forms, examples of which have been described above and further examples of which are described further below. Detailed implementation examples of the components of the device of FIG. 9 are illustrated in the subsequent drawings and described below.

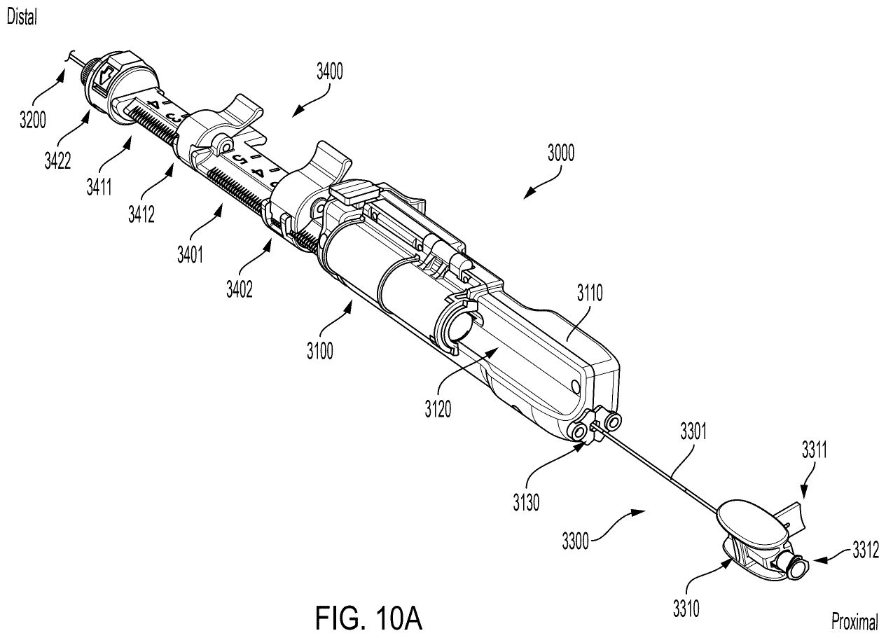

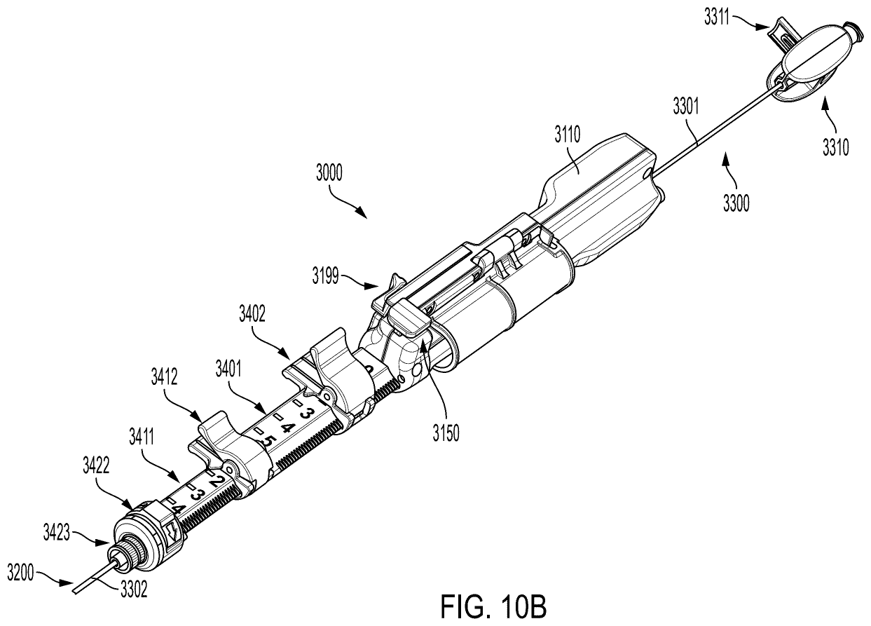

FIGS. 10A-10C are views of an example handheld infusion system 3000 according to aspects of the present disclosure. The handheld infusion system 3000 includes a handle portion 3100, an adjustment assembly 3400 coupled to the distal end of the body 3110 of the handle portion, a sheath assembly 3200 extending distally from the adjustment assembly 3400, and an inner fluid lumen assembly 3300 that extends through the body 3110 and adjustment assembly 3400 and through the sheath assembly 3200. The body 3110 includes a receptacle 3120 for a medicament container (e.g., pre-filled syringe) and a coupling mechanism 3130 for securing the inner fluid lumen assembly 3300 to the body and, as explained in more detail later, enabling distal movement of the body 3110 with respect to the adjustment assembly 3400 to distally advance a needle (not shown) at the distal end of the inner fluid lumen assembly 3300 with respect to a sheath of the sheath assembly 3200.

The adjustment assembly 3400 includes a sheath adjustment slider 3411 disposed within a needle depth adjustment slider 3401 and, at the distal end (shown in FIG. 10B) a coupling mechanism 3423 for securing the assembly 3000 to, for example, an endoscope (not shown), such that the sheath 3202 of the sheath assembly 3200 extends through a working channel of the endoscope as well as the inner fluid lumen 3301 of the inner fluid lumen assembly 3300 within the sheath 3202.

The adjustment assembly 3400 also includes a selectively lockable rotational adjustment mechanism 3422 that selectively enables rotation of the assembly 3000 with respect to an endoscope coupled to the coupling mechanism 3234. The adjustment assembly includes a sheath adjustment mechanism 3412 that selectively enables movement of the sheath adjustment slider 3411 into the needle depth adjustment slider 3401, thereby changing the length of sheath 3202 extending beyond the coupling mechanism 3234. The adjustment assembly includes a depth adjustment mechanism 3402 that can be selectively moved along the needle depth adjustment slider 3401 and thereby defines the distalmost position of the body 3110, which can slide along the needle depth adjustment slider 3401 and thereby advance and retract the inner fluid lumen 3301 (and the distal needle) with respect to the sheath 3202, as explained in more detailed herein.

The inner fluid lumen assembly 3100 includes fluid stop mechanism 3310, which in some embodiments acts as an emergency stop mechanism, at the proximal end and a Luer lock 3312 for fluidly coupling a fluid lumen (e.g., from a medicament container held by the body 3110) to the inner fluid lumen 3301. The fluid stop mechanism 3310 includes a lock body 3311 that is configured to move between a retracted position (as shown) where the inner fluid lumen is unoccluded and a locked position (as shown in FIG. 12) where the inner fluid lumen is completely blocked. In operation, the fluid stop mechanism 3310 enables an ongoing infusion to be paused as well as priming of the inner fluid lumen 3301 such that a fluid (e.g., saline) is pushed through the inner fluid lumen 3301 and then the fluid stop mechanism 3310 is engaged to hold the saline in the line before connection of a new fluid line from a medicament container prior to infusion.

The handle body 3301 includes a button 3150 that is configured to be activated (e.g., depressed) to commence an infusion, as explained in more detail below. The handle body 3301 also includes a safety 3199 (e.g., a latch, as shown) that can be selectively engaged to allow to disallow pressing of the button 3150.

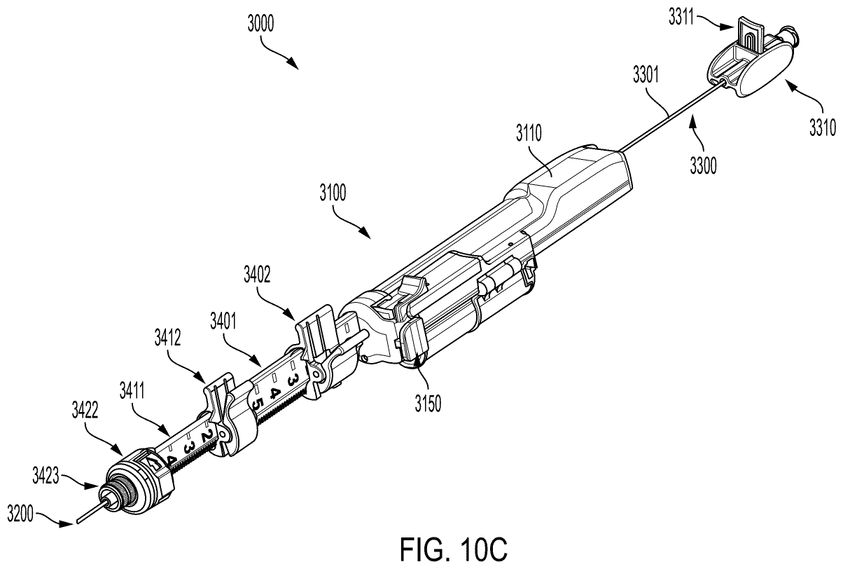

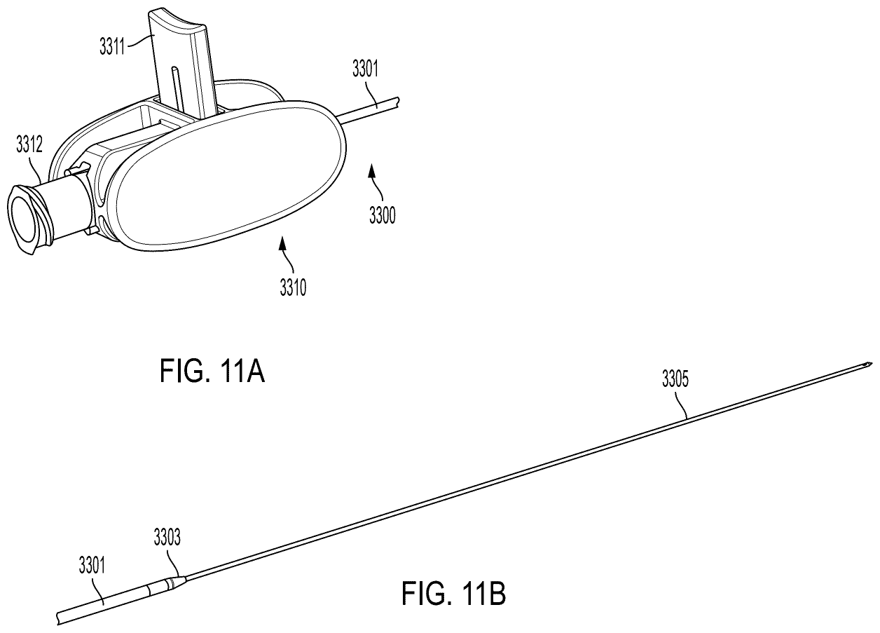

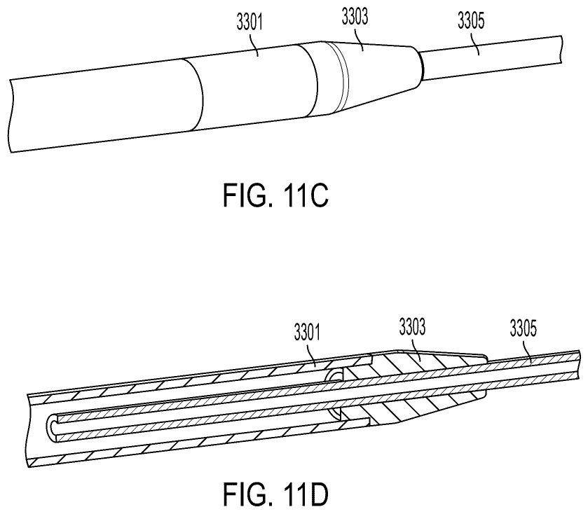

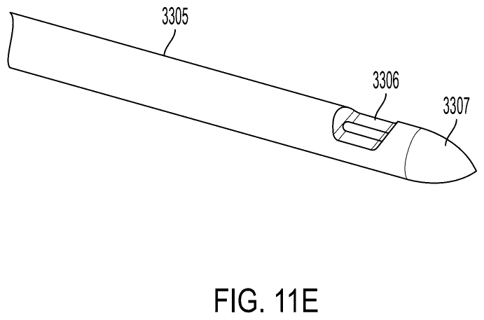

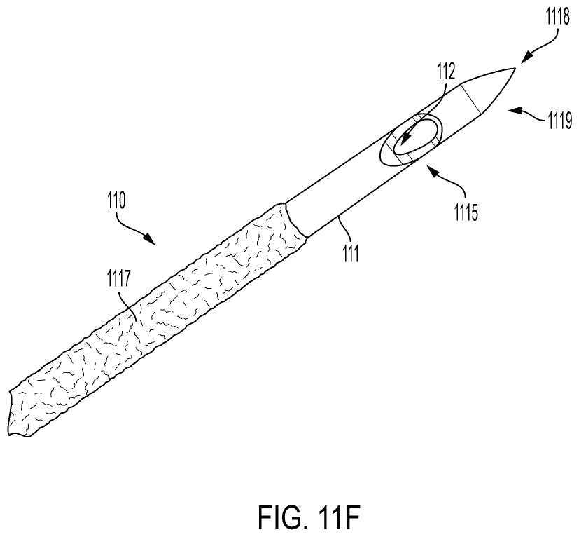

FIG. 11A shows the fluid stop mechanism 3310 of the inner lumen assembly 3300 in closer detail. FIGS. 11B-11D are view of the distal end of the inner lumen assembly 3300, and includes a distal needle 3305 extending from the inner fluid lumen 3301, with the two being connected by a conical tapered region 3303 that can include, for example, a weld between the two. The inner fluid lumen 3301 can have a two-part construction, with an inner nylon lining (for reduced viral adsorption) and a stronger (e.g., laser cut steel tube) exterior shell that enables column forces to be directed to the needle 3305 (e.g., steel needle) for puncturing tissue with the needle prior to infusion, the puncture force being provided by the handle body (3100 in FIG. 10A) advancing the inner fluid lumen 3301 distally within the sheath (3202 of FIG. 10B). Additionally, when the inner fluid lumen 3301 has a two-part construction, an inner liner (e.g., a nylon inner lining) can be, for example, thermally welded via a reflow process to the proximal end of the needle. Thereafter, the exterior of the inner fluid lumen 3301 (e.g., a laser cut steel tube) is then welded to the proximal end of the nose cone.

Examples can include a laser-cut tube (LCT) as the exterior shell of the inner fluid lumen, which is advantageous for effective handle-to-needle force translation and overall device performance. Empirical testing demonstrated that an example device using a 27 G need and incorporating an LCT requires nearly twice the handle force to generate equivalent needle force compared to a commercially available 25 G needle device. For example, a handle force of approximately 35 N yielded a needle force of 6.2 N in the example 27G device, whereas an 18 N handle force produced a needle force of 6.0 N in the off-the-shelf device. Moreover, the 27 G conical needle design required significantly less force for tissue puncture-approximately one-third of the force needed by off-the-shelf 25 G bevel needle designs-when tested in both stomach tissue and polyurethane substrates. These results indicate that a LCT-based 27 G device not only matches but exceeds the performance of a conventional 25 G needle device and is anticipated to outperform larger gauge needles such as 22 G.

While the choice of the LCT over a braided fluid lumen may have advantages, braided designs are considered. A representative example of the outer sheath can have an approximate outer diameter of 0.070 inches, while the laser-cut tube (LCT) serving as the exterior shell of the inner fluid lumen can have an approximate outer diameter of 0.042 inches. Although a braided lumen (using, for example, a PTFE or polyimide liner with a jacket) might function in similar applications, challenges in securely attaching the braided lumen to the needle and achieving the necessary thermal processing make it more difficult to integrate. Additionally, the potential for altered infusion characteristics due to texturing of the inner diameter, which could adversely affect applications such as cell injections, was a concern. In contrast, the LCT offers significant advantages: it can be readily customized to provide variable stiffness along its length (with increased flexibility proximally for syringe connection and controlled stiffness distally within the sheath), it supports precise welding to the needle, and its cut pattern can be tailored to achieve desired mechanical properties. These features collectively enhance force transmission and puncture performance while providing design flexibility for optimizing fluid delivery across a range of therapeutic applications.

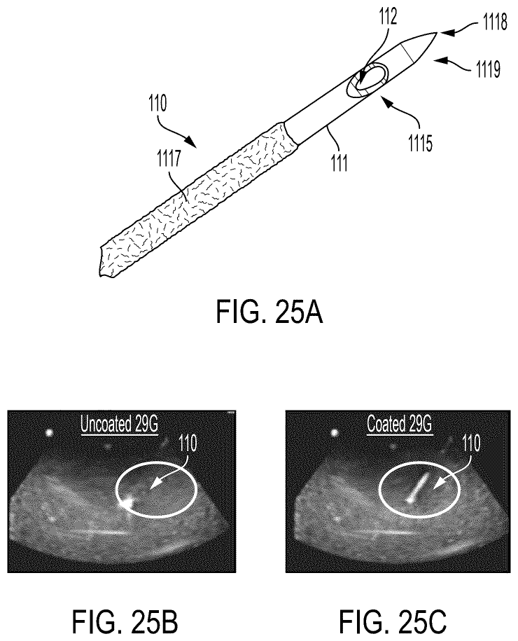

FIG. 11E is a detail view of the distal end of the distal needle 3305, showing a distal tip 3307 having a pencil tip or conical shape, which could be, for example, a tapered conical shape. In some examples, the needle 3305 is 27 G. The needle 3305 has an opening 3306 on the side which is configured to eject fluid delivered to the inner fluid lumen 3301 at the proximal end. FIG. 11F shows that the needle may include a hyperechogenic coating in at least some embodiments to facilitate visualization during ultrasound imaging. Further details about the needle according to various embodiments are described further below in connection with FIGS. 25A-25C, 26, and 27.

FIG. 12 is a partially transparent view of the fluid stop mechanism 3310, which includes a lock body 3311 configured to move within the body 3315 of the fluid stop mechanism 3310. FIG. 12 shows that the inner fluid lumen 3301 includes a proximal region 3313 that couples the inner fluid lumen 3301 to the luer lock 3313 and further that the proximal region 3313 is disposed within a cut-out 3319 of the lock body 3311. The cut-out 3319 is shaped with a wide-spaced region 3318 and a narrow region 3317 such that movement of the lock body 3311 moves the proximal region 3313 between the wide-space region 3318 and the narrow region 3317. When the proximal region 3313 is in the wide-spaced region 3318, the inner fluid lumen 3301 is unoccluded from the Luer lock 3313, and moving the locking body 3311 to dispose the proximal region 3313 in the narrow region 3317, pinches the tubing of the proximal region 3313 and completely occludes the inner fluid lumen 3301.

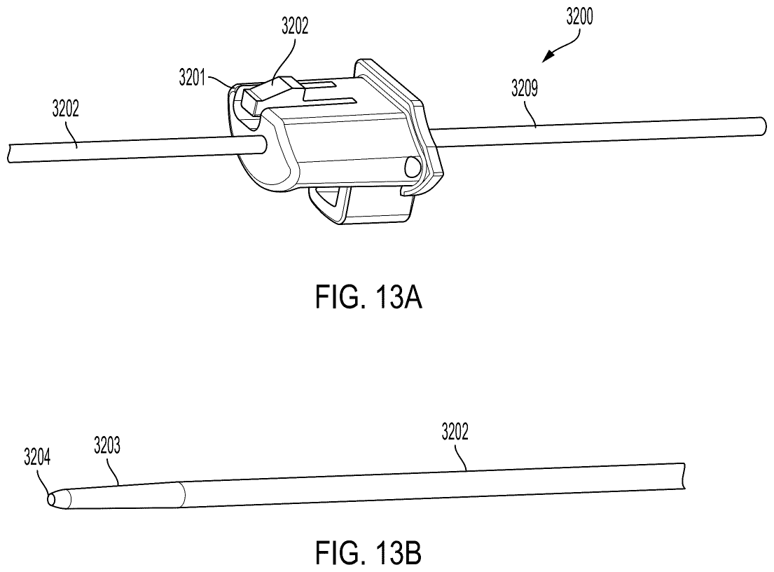

FIG. 13A is a view of a proximal end of an example outer sheath assembly 3200. The outer sheath assembly 3200 includes a connection body 3201 for securing the outer sheath assembly 3200 to the body 3110 of the handle assembly 3300. The outer sheath assembly 3200 includes a sheath 3202 that extends distally and a rigid tube 3209 that extends proximally. In operation, the body 3201 is securely held by the proximal end of the depth adjustment slider 3401 (e.g., by a clip 3202) and the rigid tube 3209 surrounds the inner fluid lumen 3301 and prevents deflection of the inner fluid lumen 3301 within the body 3110. The distal end of the outer sheath assembly 3200 is shown in FIG. 13B and includes the sheath 3202 having a tapered end portion 3202 that terminates an in opening 3204 that is configured to have the needle 3305 extend therefrom to conduct a tissue puncture and infusion operation. In operation, the majority of the sheath 3202 is disposed within the working channel of an endoscope. And, in some instances, the distal end portion 3203 is extended distally against tissue prior to distal advancement of the needle 3305 to conduct a tissue puncture prior to infusion. FIG. 13C shows the cross-section of the connection body 3201 with the connection between the sheath 3202 and the rigid tube 3209.

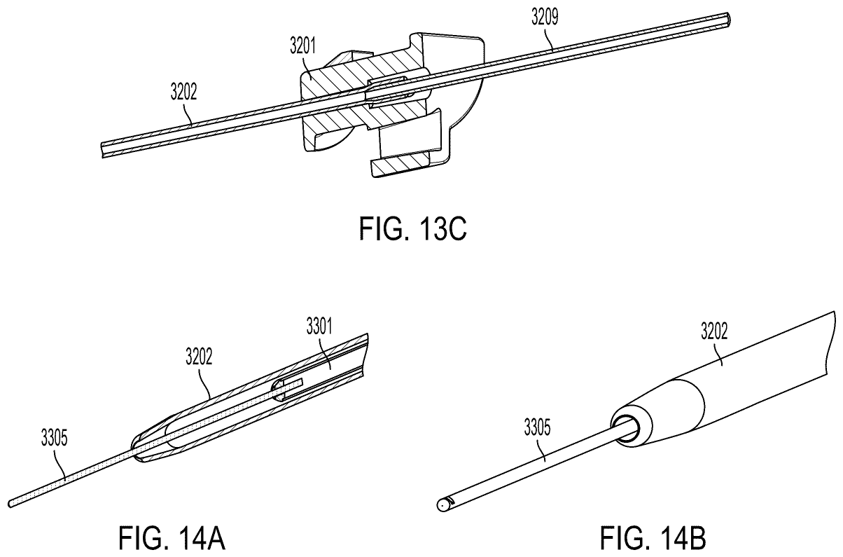

FIG. 14A is a cross-sectional view of the distal end of the inner fluid lumen assembly of FIG. 11B with the distal end of the outer sheath assembly of FIG. 13B in an operational configuration. FIG. 14B is a view of the assembly of FIG. 34A.

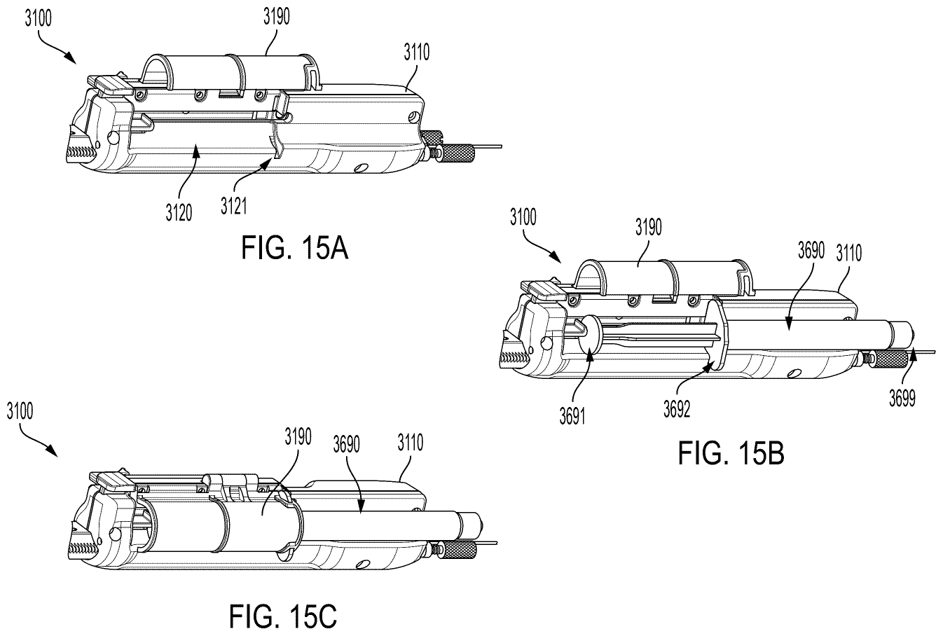

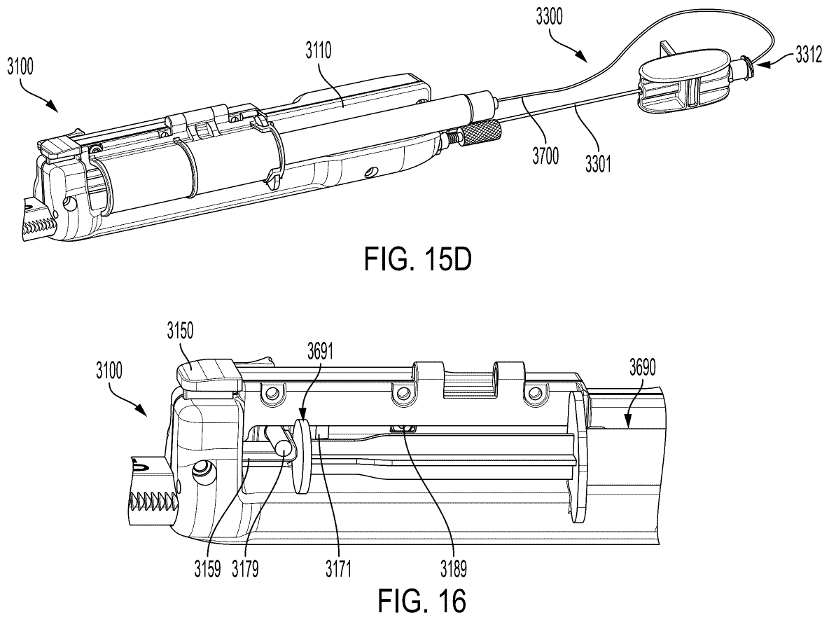

FIGS. 15A-15D are views of the handle body 3110 of the example handheld infusion system 3300 showing the installation of a medicament syringe 3690. The body 33110 includes a receptacle 3120 sized and shaped to hold a medicament container therein with the assistance of a moveable (e.g., clamshell) cover 3190 that rotates to allow the medicament container to be disposed in the receptacle 3120 (as shown in FIG. 15B) and then rotated again to prevent removal of the medicament container (as shown in FIG. 15C). In the examples of FIGS. 15A-15D, the medicament container is a syringe (e.g., a traditional pre-filled syringe) with a moveable plunger 3691 to force the fluid out of the proximal end 3699 and flanges 3692 (e.g., to facilitate hand-held use). Here, the flanges 3692 are used to longitudinally secure the syringe 3690 within the receptacle 3120 such that a drive mechanism of the body 3110 can apply a longitudinal force to the moveable plunger 3691 to drive fluid out of the syringe 3690 and conduct an infusion, with the fluid being driven through the inner fluid lumen 3301 via the proximal end 3699 (e.g., open end) of the syringe 3690 being coupled with the Luer lock 3312 of the inner fluid lumen assembly 3300, as indicated by fluid line 3700 in FIG. 15B.

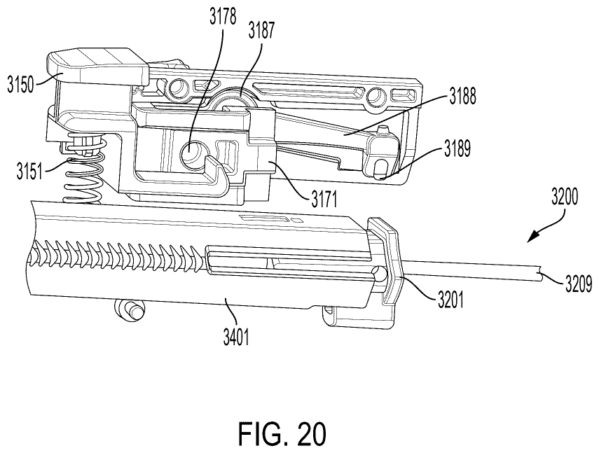

FIG. 16 shows a drive mechanism of the handle portion 3100 of the example handheld infusion system engaged with the plunger 3691 of an installed medicament syringe 3690 prior in a ready-to-use configuration. The drive mechanism includes a pin 3179 that is configured to, when released, move proximally and contact the plunger 3691 to apply a proximal force to the plunger 3691 and drive the plunger proximally into the syringe 3960. As shown in FIG. 16, the pin 3179 is held behind a latch 3159 that is disengaged laterally by depressing the button 3150 and thus commencing an infusion. The pin 3179 is attached to a carrier 3717 that is pulled proximally within the body 3110 by a force mechanism (e.g., constant force springs). As explained in more detail below, the button 3150 is also connected to a lever that selectively displaces a stop pin 3189 that is in the proximal path of the carrier 3717, such that, once released by the button 3150, the proximal movement of the pin 3179 and carrier 3171 is halted by the stop pin 3189, which marks the completion of a continuous first infusion. With the carrier 3171 contacting the stop pin 3181, and as shown in more detail in FIG. 20, a subsequent press of the button 3150 releases the carrier and frees the carrier 3171 and pin 3179 to move further proximally and conduct a second continuous infusion.

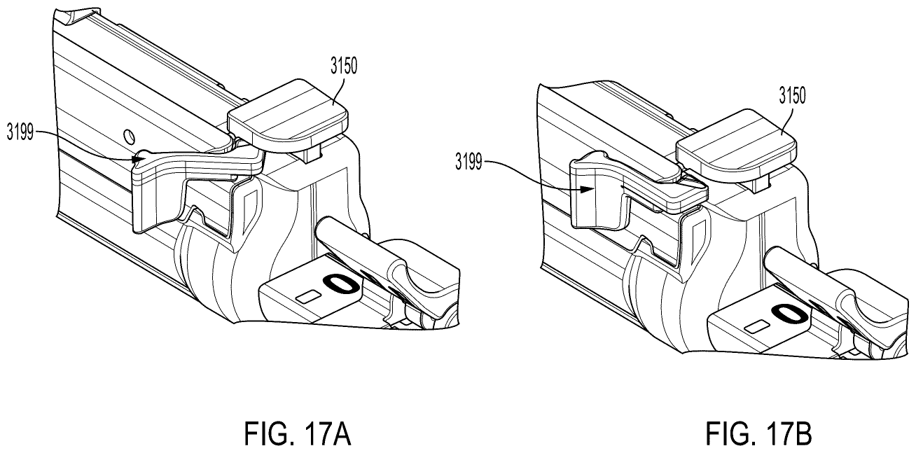

FIGS. 17A and 17B are views of the activation button 3150 and safety stop mechanism 3199 of the of the handle portion of the example handheld infusion system. In operation, the safety stop mechanism 3199 can be rotated into a position under the button 3150, as shown in FIG. 17A, to prevent actuation of the button 3150. Once actuation is desired, the safety stop mechanism 3199 can be rotated out of position, as shown in FIG. 17B, to all the button 3150 to be pressed. The safety stop mechanism 3199 can also, for example, be used to lock out an accidental depressing of the button 3150 during a first infusion such that the stopping of the carrier 3171 at the stop pin 3189 is ensured and a subsequent second infusion is not accidentally commenced until the safety stop mechanism 3199 is rotated out of the way.

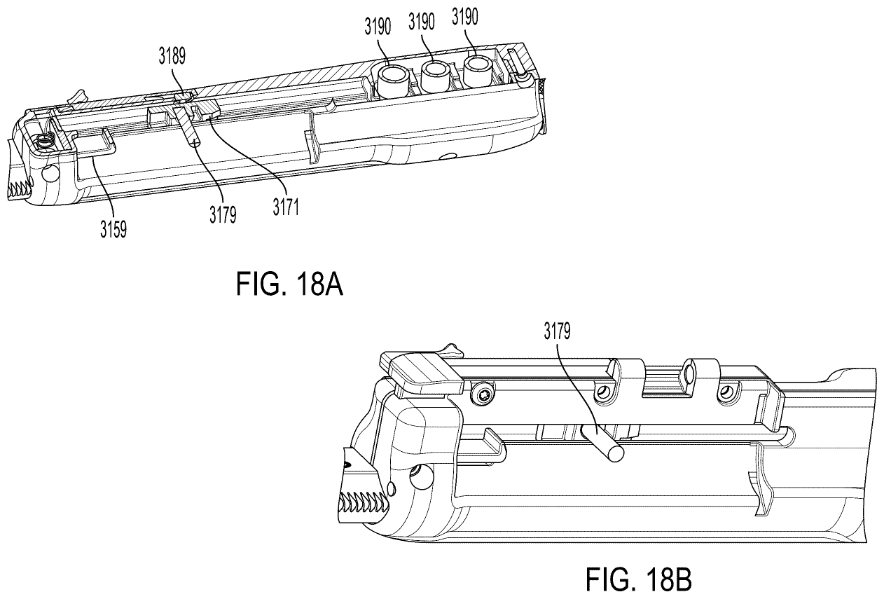

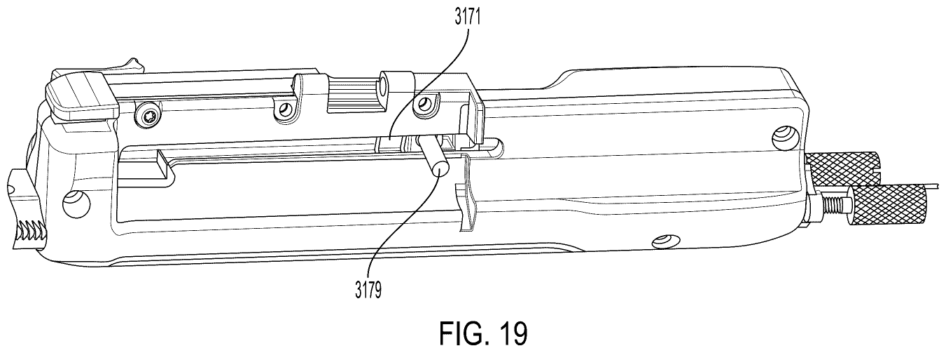

FIG. 18A is a cross-section view of the body 3110, showing more details about the drive mechanism, including a group of constant force springs 3190 configured to bias the carrier 3171 in the proximal direction. In FIGS. 18A and 18B, the carrier 3171 is in the intermediate stop position, restrained from moving further proximally by a geometric interference with a protrusion (not visible) of the carrier 3171 and the stop pin 3189. With the stop pin 3189 released (e.g., by actuating the button 3150), the carrier can proceed further proximally until it either bottoms out the carrier 3171 in the body 3110 or the plunger of the syringe reaches a fully depressed position, as represented by the position of the carrier in FIG. 19.

FIG. 20 is a view of the actuation mechanism of the handle portion showing a carrier 3171 of the drive mechanism in an initial position. The actuation mechanism includes the button 3150, the latch 3178 and a pivot arm 3188 coupled to the button 3150 at a distal end and having the stop pin 3189 at the proximal end. The pivot arm 3188 is rotably fixed about a central point 3187 and is configured to nominally dispose the stop pin 3189 in the path of the carrier 3171 and then, upon depression of the button 3150, raise the stop pin 3189 out of the path the carrier 3717 to permit the carrier 3717 to proceed to translate proximally (e.g., a second infusion). The button 3150 is biased by a spring 3151, which also ensures that the stop pin 3189 maintains the nominally obstructive position and prevents accidental displacement of the stop pin 3189 against the proximal bias of the carrier 3171 by the force mechanism (e.g., the constant force springs).

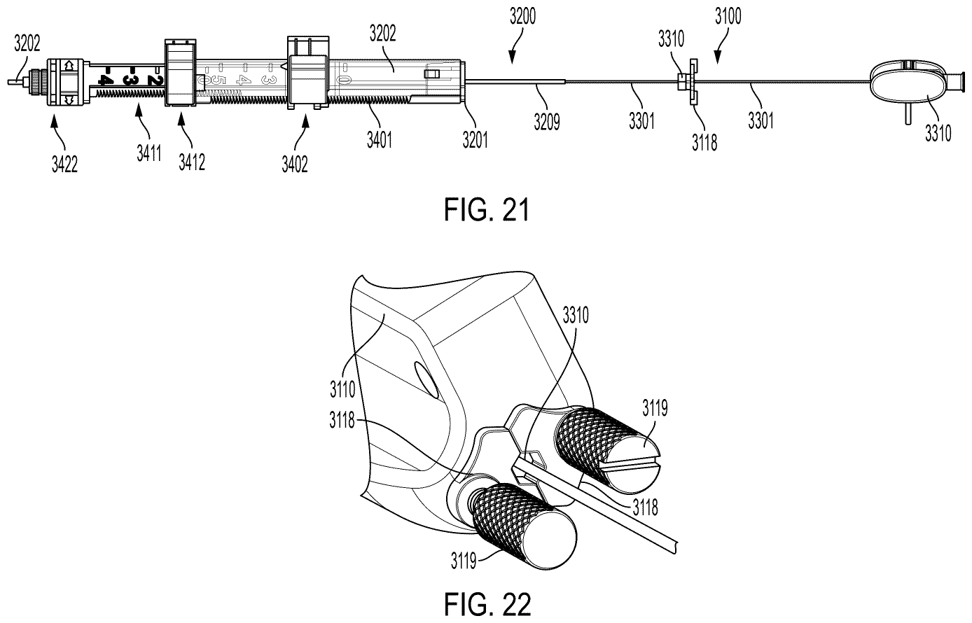

Also shown in FIG. 20, is the attachment of the connection body 3201 of the sheath assembly 3200 to the proximal end of the depth adjustment slider 3401, with the arrangement of the sheath assembly 3200 and the inner fluid lumen assembly 3300 shown in more detail in FIG. 21. In FIG. 21, the entire assembly 3000 is shown with the body 3110 of the handle portion removed. Notably, the thrust flange 3310 of the inner fluid lumen assembly is shown, which is configured to be fixedly secured to the body 3110 by removeable plates 3118 (as shown in more detail in FIG. 22) and transfer proximal and distal forces to the inner fluid lumen 3301 (e.g., by providing a force to the exterior shell of the inner fluid lumen, which can be a flexible tube with significantly higher column strength than the inner lining, such as a laser cut stainless steel tube). In some examples, the exterior shell ends at the thrust flange with only the flexible inner lining extending proximally to the fluid stop mechanism 3310 to enable, in some instances, the Luer lock of the fluid stop mechanism 3310 to be directly connect to the syringe. In other instances, the same arrangement is made with the exterior shell extending fully or partially to the fluid stop mechanism 3310 and being flexible enough to permit the connection of the fluid stop mechanism 3310 directly to the syringe. FIG. 22 shows the proximal end of the body 3110 includes two plates 3118 securing the thrust flange 3310 inside the body 3110, each with a thumb screw 3119 for securing the plate 3118 in place. In some examples, the thrust flange 3310 is shaped such that rotation with respect to the body 3110 is constrained (e.g., having a hexagonal exterior profile disposed with a corresponding socket within the body 3100).

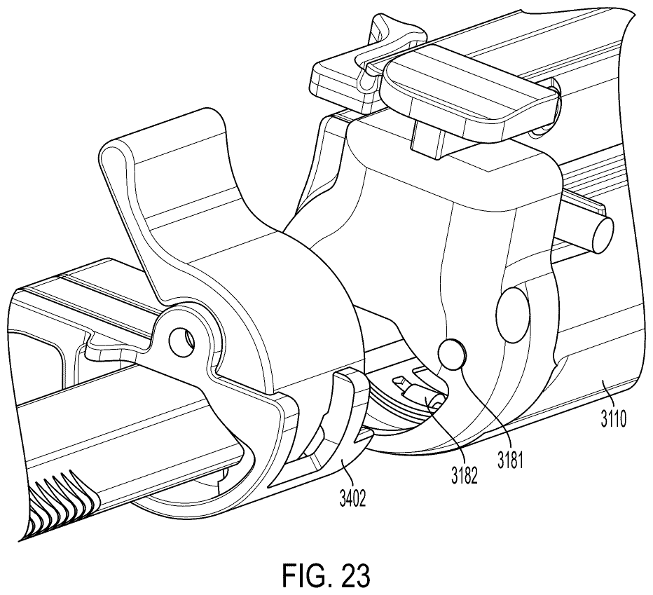

FIG. 23 is a view of the distal end of the body portion with the sheath adjustment slider removed to show the magnetic attachment element on the proximal end of the body and the friction roller inside the body. The magnetic attachment element 3181 is configured to magnetically engage with a corresponding magnet on the depth adjustment mechanism 3402 such that, when the body 3110 is advanced distally against the depth adjustment mechanism 3402 (e.g., to conduct a needle puncture from the distal end of the assembly), the body 3110 is magnetically restrained from easily moving proximally away from the depth adjustment mechanism 3402 and thereby reduces the chances of accidentally withdrawing the needle by adding a magnetic attractive force to overcome. Additionally, or alternatively, examples include the use of a friction roller 3182 inside the body and configured to be rolled by movement of the depth adjustment slide (not shown) within the body 3110, thereby adding an additional force to overcome to induce movement of the needle with respect to the sheath.

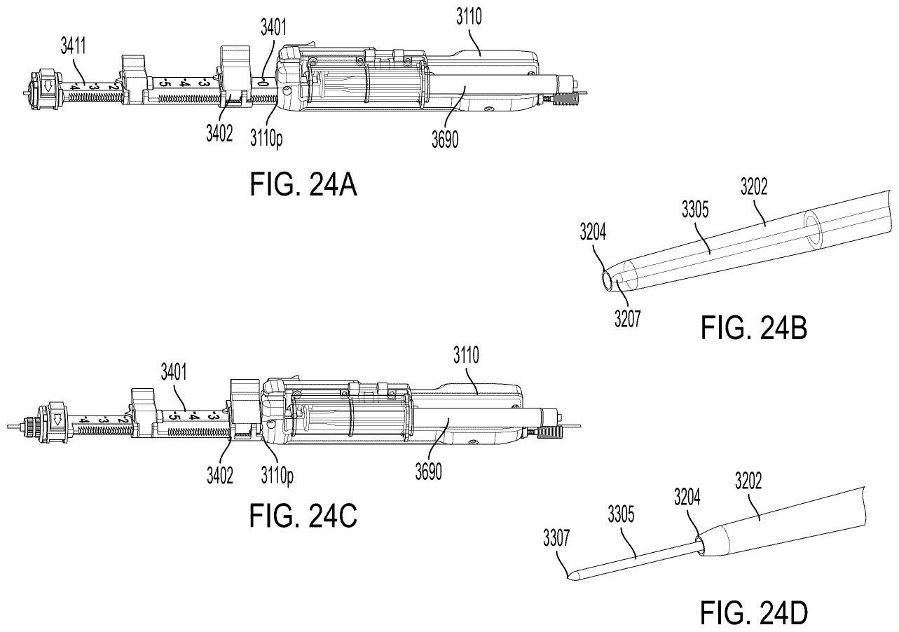

FIGS. 24A-24D show details of the needle movement that is caused by movement of the body 3110 with respect to the depth adjustment slider 3402. FIG. 24A shows the depth adjustment mechanism 3402 moved to a set at a specific location along the depth adjustment slider 3402, the distance of the depth adjustment slider 3402 from the proximal end of the body 3110 defining a corresponding maximum distal translation of the needle 3305. In operation, the initial relationship between the sheath 3202 and the needle 3305 is fixed because the distance between the thrust flange 3310 of the inner fluid lumen assembly 3300 and the connection body 3201 of the sheath assembly 3200 is fixed by the body (e.g., the body holds the thrust flange and the depth adjustment slider 3402, to which the connection body 3201 is coupled, is maximally distally extended from the body). In some examples, and as shown in FIG. 24B, the fixed initial position of the needle 3505 within the sheath 3202 is fully within the sheath 3202. FIG. 24C shows the handle being displaced distally against the depth adjustment mechanism 3402, understanding that the depth adjustment slide 3401 is secured to the endoscope via the sheath adjustment slider 3411). The distal movement of the body 3110 causes a corresponding distal advancement of the needle 3305 with respect to the sheath 3202, as shown in FIG. 24D.

Referring now to FIGS. 25A-25C, a photograph of the distal portion of a shaft including a hyperechogenic coating, and examples of coated and uncoated needles as viewed using ultrasonic imaging are illustrated, respectively, consistent with the present inventive concepts. Infusion assembly 110 and other components of FIGS. 25A-25C can be of similar construction and arrangement to similar components described in other figures herein. As described herein, infusion assembly 110 can comprise shaft 111, such as a needle-like shaft, with lumen 112 passing therethrough. In some embodiments, a tip-portion of shaft 111, tip 1119, can comprise a “pencil-point” geometry, as shown, such as when shaft 111 comprises a conical distal end. Alternatively or additionally, tip 1119 can comprise a beveled geometry, such as a geometry selected from the group consisting of: a long bevel; a medium bevel; a short bevel; a multi-bevel; a scalpel bevel; and combinations of these. The distal portion of shaft 111 can comprise one or more holes or other openings (e.g., an array of holes), such as port 1115 shown. Port 1115 can comprise one, two, three or more openings through a side wall of shaft 111, such that lumen 112 exits the side wall of shaft 111 at a location proximal to distal end 1118 (e.g., and a location proximal to tip 1119), also as shown. Port 1115 can exit the side of shaft 111, as shown, and/or port 1115 can exit distal end 1118 of shaft 111. In some embodiments, port 1115 can comprise two, three, or more side facing openings (e.g., openings through the side wall of shaft 111), such as multiple openings positioned circumferentially about shaft 111 and/or multiple openings positioned along a length of the distal portion of shaft 111. In some embodiments, shaft 111 can comprise one or more porous segments, such as a porous segment of shaft 111 positioned near the distal portion of shaft 111. The porous segment can be configured as one or more ports 1115 that are constructed and arranged such that material 60 can exit lumen 112 via the porosity of the segment. Port 1115 can comprise an opening with a cross sectional geometry selected from the group consisting of: round; oval; rectangular; trapezoidal; and combinations of these. In some embodiments, each port 1115 comprises a cross-sectional area of no less than the cross-sectional area of lumen 112, such as at least 25% larger than the cross-sectional area of lumen 112. Alternatively or additionally, the total cross-sectional area of all of ports 1115 (e.g., when shaft 111 comprises two or more ports 1115) is no less than the cross-sectional area of lumen 112, such as at least 25% larger than the cross-sectional area of lumen 112. In some embodiments, port 1115 comprises a major diameter of at least 0.4 cm, such as at least 0.6 cm, 0.8 cm, or 1.0 cm. In some embodiments, the distal end of port 1115 (e.g., the distal end of the most distal port 1115 when port 1115 comprises multiple ports) is less than 1 cm from distal end 1118 of shaft 111, such as less than 0.8 cm, 0.6 cm, of 0.4 cm.

One or more portions of shaft 111 can each comprise one, two, or more coatings, other surface treatments, and/or other surface modifications, coating 1117 shown. Coating 1117 can comprise a coating configured to increase the visibility of shaft 111 when imaged using one or more imaging modalities. For example, coating 1117 can comprise a hyperechogenic coating configured to increase the echogenicity of shaft 111 when imaged using ultrasonic imaging. Alternatively or additionally, coating 1117 can comprise a radiopaque coating configured to increase the visibility using X-ray imaging. FIGS. 25B and 25C show examples of uncoated and coated, respectively, 29 G needles imaged using ultrasonic imaging. As can be seen in the figures, coating 1117 increases the visibility of shaft 111 in FIG. 25C compared to the otherwise similar shaft 111 shown in FIG. 25B. In some embodiments, coating 1117 comprises an hyperechogenic coating such as Sono-Coat™ provided by Encapson B.V. In some embodiments, coating 1117 can comprise a biocompatible coating and/or a temperature-resistant coating, such as a coating configured to withstand temperatures of at least 100° C. without adversely affecting the coating. In some embodiments, coating 1117 can include two or more coatings, such as when coating 1117 comprises both a hyperechogenic coating and a second coating overcoated on the hyperechogenic coating, such as when the second coating comprises a lubricious, anti-microbial, and/or anti-thrombogenic coating. In some embodiments, the overcoating of coating 1117 does not decrease the echogenicity provided by the hyperechogenic coating 1117. In some embodiments, coating 1117 comprises a thickness of no more than 0.015 mm, such as less than 0.03 mm, 0.06 mm, or 0.08 mm.

In some embodiments, shaft 111 comprises a shaft with a diameter no larger than 25 G, such as a diameter of no more than 26 G, 27 G, 28 G, 29 G, or 30 G. One or more surface and/or other portions of shaft 111 can comprise coating 1117. In some embodiments, coating 1117 is applied to the outer surface of shaft 111 proximate but proximal to port 1115 as shown. The distal end of coating 1117 can terminate at a location that is no more than 1.00 cm from distal end 1118 of shaft 111, such as no more than 0.75 cm, 0.50 cm, or 0.25 cm. In some embodiments, coating 1117 comprises a length of at least 0.25 cm, such as at least 0.50 cm, 0.75 cm, or 1.00 cm. Additionally or alternatively, coating 1117 can comprise a length of no more than 1.50 cm, such as no more than 1.25 cm, 1.00 cm, or 0.75 cm. As described herein, shaft 111 can comprise one, two, or more materials, such as stainless steel, a cobalt-chromium alloy, a nickel-titanium alloy, and/or a non-metallic material, such as PEEK.

In some embodiments, infusion assembly 110 can include a single-part shaft (e.g., shaft 111 comprises a single-part shaft), such as when shaft 111 comprises a single-part shaft that extends the length of depositing device 100 (not shown, but described herein), such as when shaft 111 is configured to be inserted into shaft 131 of positioning assembly 130, to pass through proximal portion 1312, and to exit shaft 131 proximate tissue to be infused with material 60. Alternatively or additionally, infusion assembly 110 can comprise a multi-part shaft (e.g., shaft 111 comprises a multi-part shaft). For example, a distal shaft of shaft 111 can comprise a needle-like shaft as described herein, and a proximal shaft of shaft 111 can comprise a tube that extends proximally from the distal shaft. The distal shaft can comprise a length of at least 5 cm, such as at least 6 cm, 7 cm, or 8 cm. A proximal shaft of shaft 111 can comprise a similar and/or a dissimilar material from a distal shaft of shaft 111. For example, a proximal shaft of shaft 111 can comprise a material that minimizes adsorption of material 60, such as when shaft 111 comprises a material that minimizes viral adsorption (e.g., when material 60 comprises a viral vector to be delivered to the tissue). For example, shaft 111 (e.g., a proximal shaft of shaft 111) can comprise a material including cyclic olefin polymers (COP) and/or other materials configured to minimize adsorption. In some embodiments, a proximal shaft of shaft 111 comprises a larger diameter (e.g., a larger inner diameter and/or a larger outer diameter) than the diameter of a distal shaft of shaft 111, for example the proximal shaft can comprise a shaft at least 1 G larger than the distal shaft, such as at least 2 G, or 4 G larger.

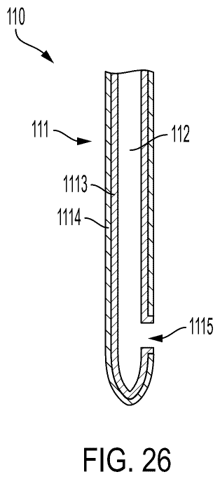

Referring now to FIG. 26, a sectional view of an embodiment of an infusion assembly is illustrated, consistent with the present inventive concepts. Infusion assembly 110 and other components of FIG. 26 can be of similar construction and arrangement to similar components described in reference to FIG. 1 and otherwise herein. Infusion assembly 110 can be configured to prevent and/or at least limit viral loss, such as viral adsorption, as treatment material 60 (not shown but described herein) is delivered to the patient via infusion assembly 110, as described herein. Infusion assembly 110 can comprise shaft 111, such as a needle-like shaft, with lumen 112 passing therethrough. In some embodiments, shaft 111 comprises a multi-layer shaft, such as a shaft including at least inner layer 1113, and two or more outer layers, outer layer 1114 shown.

In some embodiments, the distal portion of shaft 111 comprises one or more holes or other openings (e.g., an array of holes), such as port 1115 shown and described herein. In some embodiments, shaft 111 comprises a blunted tip, such as a “bullet” or a “pencil point” tip, as shown and described herein. Port 1115 can comprise a hole through a side wall of shaft 111.

Inner layer 1113 can comprise at least the inner surface of the walls of lumen 112. In some embodiments, inner layer 1113 is constructed and arranged to limit viral loss, such as when inner layer 1113 comprises a material that is selected to prevent viral loss (e.g., to limit adsorption of treatment material 60 to the walls of inner layer 1113). For example, inner layer 1113 can comprise a material selected from the group consisting of: borosilicate glass; high density polyethylene (HDPE); low density polyethylene (LDPE); crystal zenith; flint glass; polycarbonate (PC); silicone; polypropylene (PP); a PP copolymer; polyethylene terephthalate glycol (PETG); polyolefin; polystyrene; a nylon material; other engineered plastics; acrylic; ABS; PLA; polyvinylidene fluoride; soda-lime glass; lead glass; aluminosilicate glass; silica glass, such as 96% silica glass; fused silica glass; ceramic glass; Saint-Gobain's Tygon® thermoplastic; PEEK; and combinations of these.

In some embodiments, inner layer 1113 comprises a coating of a material, such as a coating that is applied to the luminal surface of lumen 112 extending through outer layer 1114. Inner layer 1113 comprising a coating can be applied to the inner walls of outer layer 1114 of shaft 111 during a manufacturing process and/or in a procedural step where infusion assembly 110 is prepared for delivery of treatment material 60 to the patient. In some embodiments, inner layer 1113 comprising a coating can comprise an anti-adhesion material, such as polyionic hydrophilic complex.

In some embodiments, system 10 includes two or more variations of infusion assembly 110, for example different variations where inner layer 1113 comprises a material that is selected to minimize viral loss based on the viral composition of treatment material 60 (e.g., a first variation of infusion assembly 110 for delivery of a first virus, and a second variation of infusion assembly 110 for delivery of a second virus). In some embodiments, treatment material 60 comprises one or more additives, additive 61 described herein. Additive 61 can be configured to limit viral loss of treatment material 60, such as viral loss due to viral adsorption. For example, additive 61 can comprise a buffer solution, such as a buffer solution selected from the group consisting of: poloxamer 188 (P188); polysorbate 20 (PS20); polysorbate 80 (PS80); and combinations of these. In some embodiments, additive 61 can comprise a biocompatible block copolymer with surface-active properties that is added to treatment material 60 as an excipient.

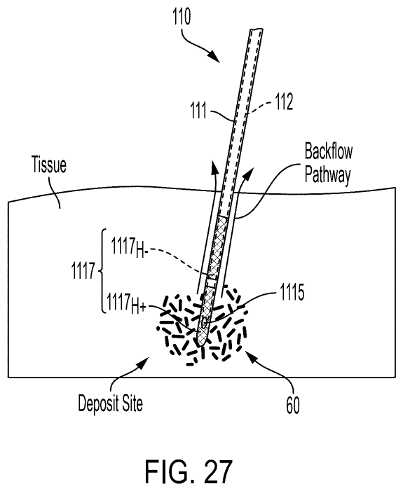

Referring now to FIG. 27, an anatomic view of an embodiment of an infusion device inserted into tissue is illustrated, consistent with the present inventive concepts. Infusion assembly 110 and other components of FIG. 27 can be of similar construction and arrangement to similar components described in reference to FIG. 1 and otherwise herein. Infusion assembly 110 can be configured to prevent and/or at least limit backflow of a material, such as treatment material 60, that is delivered to the patient via infusion assembly 110 (e.g., infused into a deposit site). When infusing a material to a deposit site to treat target tissue (e.g., tissue of an organ of a patient), backflow can occur when the material exits the organ via a fluid path surrounding shaft 111 (e.g., the path of least resistance for treatment material 60 can comprise a fluid path between the tissue surrounding shaft 111 and shaft 111, such as a path created when shaft 111 is inserted through the tissue). In some embodiments, system 10 can be configured to limit backflow (e.g., in addition to anti-backflow configurations of infusion assembly 110), as described herein. The pathway created by the insertion of shaft 111 through the tissue can be referred to herein as a “backflow pathway”. While shaft 111 is positioned within the tissue (e.g., before shaft 111 is retracted after an infusion of treatment material 60), the backflow pathway comprises an initial backflow pathway BP1 comprising the fluid path between the outer surface of shaft 111 and the surrounding tissue as described hereabove. Once shaft 111 has been removed, the backflow pathway comprises a remaining pathway BP2 comprising the fluid path that remains due to tissue disruption caused by the insertion and/or removal of shaft 111.

In further example embodiments of the present inventive concepts, in addition to or as an alternative to structural anti-backflow features of infusion assembly 110, system 10 includes one or more system-level backflow mitigation strategies configured to reduce formation and/or utilization of the backflow pathway BP1 and/or the remaining pathway BP2. In certain embodiments, the system is configured to control an infusion pressure profile to reduce backflow, such as by implementing a pressure ramp at the start of infusion (e.g., gradually increasing pressure and/or flow rate from a first level to a target level over a predetermined ramp interval) to reduce sudden jetting and minimize enlargement of BP1. In further embodiments, system 10 is configured to perform post-infusion dwell control, such that after a target volume is delivered the system maintains the needle position within the tissue for a dwell period while allowing pressure to decay in a controlled manner (e.g., by partially restricting the fluid lumen, by permitting controlled venting, or by actively maintaining a low holding pressure) so that tissue can relax and seal around shaft 111 and the deposit can equilibrate within the target compartment, thereby reducing driving pressure for backflow along BP1. In still further embodiments, system 10 is configured to implement a needle withdrawal sequencing protocol, such as retracting shaft 111 in a staged manner (e.g., retract to a first intermediate position and pause, then retract fully) and/or coordinating retraction with a fluid stop or partial occlusion state, thereby reducing the likelihood that residual pressure or tissue recoil causes treatment material 60 to be expressed along BP1 during withdrawal and reducing residual leakage along BP2 after removal. In some embodiments, the system-level strategies are executed automatically by a controller based on measured or inferred delivery parameters (e.g., syringe pressure, flow rate, and/or tissue compliance proxies) and/or based on user selection of an infusion mode, and may be combined with operator prompts (e.g., “hold position,” “dwell complete,” “withdraw in steps”) to standardize technique and further mitigate backflow.

Infusion assembly 110 can comprise shaft 111, such as a needle-like shaft, with lumen 112 passing therethrough. As described herein, infusion assembly 110 can comprise coating 1117. Coating 1117 can comprise one or more coatings, such as one or more coatings applied to one or more segments of shaft 111, such as one or more discrete and/or overlapping segments. Infusion assembly 110 can include port 1115, such as one or more side ports, for example as described in reference to FIG. 21 and otherwise herein.

In some embodiments, coating 1117 comprises a hydrophobic coating, coating 1117H—, that surrounds (e.g., has been applied to the outer surface of) a segment of shaft 111 proximal to port 1115. Coating 1117H—can comprise a hydrophobic coating selected from the group consisting of: a PTFE coating; a silicone coating; another hydrophobic material coating; and combinations of these. Coating 1117H—can be configured to limit treatment material 60 from following the initial backflow pathway proximally along shaft 111. In some embodiments, the portion of shaft 111 that is distal to the portion comprising coating 1117H—is more hydrophilic than the portion comprising coating 1117H—, such that treatment material 60 is more likely to “cling” to the distal segment, and to infuse into tissue (e.g., instead of following the initial backflow pathway). In some embodiments, coating 1117 comprises a hydrophilic coating, coating 1117H+. For example, shaft 111 can comprise a proximal segment (e.g., proximal to port 1115) comprising hydrophobic coating 1117H—, and a more distal segment (e.g., a segment distal to and/or including port 1115) comprising hydrophilic coating 1117H+. Coating 1117H+ can further increase the “cling” of treatment material 60 to the distal portion of shaft 111 (e.g., to maintain treatment material 60 proximate the target region and limit backflow while treatment material 60 is infused into the target region).

In some embodiments, application of coating 1117 (e.g., in a manufacturing process) comprises a surface treatment that is performed on a segment of shaft 111. In some embodiments, application of coating 1117 can include hydrofluoric acid etching and/or fluorosilane modification. For example, application of coating 1117 can include a pretreatment configured to increase the surface roughness of shaft 111 (e.g., hydrofluoric acid etching), followed by covalent modification with long-chain fluorosilanes, such as to form a self-assembled monolayer (SAM). In some embodiments, coating 1117, such as coating 1117 comprising a SAM coating, is configured to reduce the surface energy of shaft 111 (e.g., shaft 111 comprising as stainless-steel shaft). Coating 1117 can comprise one or more surface modifications, such as one or more modifications selected from the group consisting of: laser-induced periodic surface structures (LIPSS); a modification configured to produce nanospikes, such as by controlling the wetting state of stainless-steel with accumulated laser fluence; and combinations of these. In some embodiments, coating 1117 comprises a coating that is applied to shaft 111 during a clinical procedure. For example, shaft 111 can be “dipped” into a coating material prior to insertion of shaft 111 into the patient. In some embodiments, coating 1117 comprises a fluid that is applied to shaft 111 prior to insertion, such as a fluid comprising a microbubble contrast agent.

In some embodiments, coating 1117 comprises a friction reducing coating, such as hydrophobic coating 1117H-described herein, or other friction reducing coating. A friction reducing coating can prevent or at least limit trauma to tissue as shaft 111 is inserted therethrough, such as to position port 1115 proximate a deposit site. One or more components and/or methods of system 10 can be configured to limit trauma to tissue, such as to prevent and/or at least limit likelihood of procedural complication, such as pancreatitis caused by tissue injury, and/or to limit the size and/or the lasting duration of the temporary backflow pathway that remains when infusion assembly 110 is removed from tissue (e.g., shaft 111 is retracted from the deposit site where treatment material 60 was delivered).

### Additional Embodiments

Additional embodiments of the disclosure are described in the numbered paragraphs below:
1. A method for preventing weight gain in a subject, comprising:
injecting pancreatic tissue of the subject with an effective amount of an adeno-associated virus (AAV) vector comprising (i) a recombinant AAV genome comprising a pancreatic islet cell specific promoter operably linked to a transgene comprising a GLP-1 receptor agonist coding sequence and (ii) AAV capsid proteins, optionally AAV9 capsid proteins, wherein the subject has a body mass index (BMI) of less than 30 kg/m².
2. The method of paragraph 1, wherein the subject has a BMI of less than 25 kg/m².
3. The method of paragraph 2, wherein the subject has a BMI of 18.5-24.9 kg/m².
4. The method of any of paragraphs 1-3, wherein the effective amount is sufficient for the subject to maintain a BMI below the treatment BMI +/−about 5% to about 10%.
5. The method of paragraph 4, wherein the effective amount is sufficient for the subject to maintain a BMI of less than 30 kg/m² on a high caloric diet for at least 7 days, at least 14 days, at least 21 days, or at least 28 days.
6. The method of paragraph 5, wherein the effective amount is sufficient for the subject to maintain a BMI of less than 30 kg/m² on a high caloric diet for at least 1 year.
7. The method of paragraph 6, wherein the effective amount is sufficient for the subject to maintain a BMI of less than 30 kg/m² on a high caloric diet for at least 2 years, or at least 5 years.
8. The method of any of paragraphs 1-7, wherein the recombinant AAV genome comprises in the following 5′ to 3′ order: a first AAV inverted terminal repeat (ITR) sequence, the pancreatic islet cell specific promoter, the transgene, a polyadenylation (polyA) sequence, and a second AAV ITR.
9. The method of any of paragraphs 8-11, wherein the GLP-1 receptor agonist coding sequence is a GLP-1₍₁₋₃₇₎ peptide coding sequence.
10. The method of paragraph 8 or 9, wherein: the first AAV inverted terminal repeat (ITR) sequence comprises the sequence of SEQ ID NO: 11, the pancreatic islet cell specific promoter comprises the sequence of SEQ ID NO: 6, the GLP-1 receptor agonist coding sequence of the transgene comprises the sequence of SEQ ID NO: 7, the WPRE comprises the sequence of SEQ ID NO: 9, the polyadenylation (polyA) sequence comprising the sequence of SEQ ID NO: 10, and the second AAV ITR comprising the sequence of SEQ ID NO: 12.
11. The method of any of paragraphs 1-10, wherein the pancreatic islet cell specific promoter comprises a human insulin promoter sequence.
12. The method of paragraph 11, wherein the human insulin promoter sequence comprises the sequence of SEQ ID NO: 6.
13. The method of any of paragraphs 1-12, wherein the transgene further comprises a human insulin signal peptide, optionally upstream from the GLP-1 receptor agonist coding sequence.
14. The method of paragraph 13, wherein the human insulin signal peptide comprises the sequence of SEQ ID NO: 13.
15. The method of any of paragraphs 1-14, wherein the transgene further comprises an intervening peptide 2 (IP2) peptide coding sequence downstream from the GLP-1 receptor agonist coding sequence.
16. The method of paragraph 15, wherein the IP2 peptide coding sequence comprises the sequence of SEQ ID NO: 8.
17. The method of any of paragraphs 1-16, wherein the transgene comprises the sequence of SEQ ID NO: 1.
18. The method of any of paragraphs 1-17, wherein the recombinant AAV genome further comprises a Woodchuck Hepatitis Virus Posttranscriptional Regulatory Element (WPRE) sequence, optionally downstream from the transgene and upstream from the polyA sequence.
19. The method of paragraph 18, wherein the WPRE sequence comprises the sequence of SEQ ID NO: 9.
20. The method of any of paragraphs 8-19, wherein the recombinant AAV genome comprises the sequence of SEQ ID NO: 2.
21. The method of any of paragraphs 8-19, wherein the recombinant AAV genome comprises the sequence of SEQ ID NO: 3.
22. The method of any of paragraphs 1-21, wherein the effective amount is about 5×10¹² to about 5×10¹⁴ AAV vector genomes (VG).
23. The method of any of paragraphs 1-22, wherein the injecting of the pancreatic tissue is via endoscopic ultrasound-guided fine needle injection (EUS-FNI).
24. The method of any of paragraphs 1-23, wherein the injecting of the pancreatic tissue comprises a single infusion.
25. The method of any of paragraphs 1-23, wherein the injecting of the pancreatic tissue comprises multiple infusions.
26. The method of paragraph 25, wherein the injecting of the pancreatic tissue comprises at least three infusions.
27. The method of any of paragraphs 1-26, wherein the pancreatic tissue is in a tail region of the pancreas.
28. The method of any of paragraphs 1-27, wherein the pancreatic tissue is in a body region of the pancreas.
29. A method for treating obesity in a subject, comprising:
injecting pancreatic tissue of the subject with an effective amount of an adeno-associated virus (AAV) vector comprising (i) a recombinant AAV genome comprising a pancreatic islet cell specific promoter operably linked to a transgene comprising a GLP-1 receptor agonist coding sequence and (ii) AAV capsid proteins, optionally AAV9 capsid proteins, wherein the subject has a body mass index (BMI) of at least 30 kg/m².
30. The method of paragraph 29, wherein the effective amount is sufficient to maintain a clinically significant reduction in body weight for at least 3 months.
31. The method of paragraph 30, wherein the effective amount is sufficient to maintain a clinically significant reduction in body weight for at least 6 months.
32. The method of paragraph 31, wherein the effective amount is sufficient to maintain a clinically significant reduction in body weight for at least 9 months.
33. The method of paragraph 32, wherein the effective amount is sufficient to maintain a clinically significant reduction in body weight for at least 1 year.
34. The method of paragraph 33, wherein the effective amount is sufficient for the subject to maintain a BMI of less than 30 kg/m² on a high caloric diet for at 3 months.
35. The method of paragraph 34, wherein the effective amount is sufficient for the subject to maintain a BMI of less than 30 kg/m² on a high caloric diet for at 6 months.
36. The method of any of paragraphs 29-35, wherein the recombinant AAV genome comprises in the following 5′ to 3′ order: a first AAV inverted terminal repeat (ITR) sequence, the pancreatic islet cell specific promoter, the transgene, a polyadenylation (polyA) sequence, and a second AAV ITR.
37. The method of any of paragraphs 36-39, wherein the GLP-1 receptor agonist coding sequence is a GLP-1₍₁₋₃₇₎ peptide coding sequence.
38. The method of paragraph 36 or 37, wherein: the first AAV inverted terminal repeat (ITR) sequence comprises the sequence of SEQ ID NO: 11, the pancreatic islet cell specific promoter comprises the sequence of SEQ ID NO: 6, the GLP-1 receptor agonist coding sequence of the transgene comprises the sequence of SEQ ID NO: 7, the WPRE comprises the sequence of SEQ ID NO: 9, the polyadenylation (polyA) sequence comprising the sequence of SEQ ID NO: 10, and the second AAV ITR comprising the sequence of SEQ ID NO: 12.
39. The method of any of paragraphs 29-38, wherein the pancreatic islet cell specific promoter comprises a human insulin promoter sequence.
40. The method of paragraph 39, wherein the human insulin promoter sequence comprises the sequence of SEQ ID NO: 6.
41. The method of any of paragraphs 29-40, wherein the transgene further comprises a human insulin signal peptide, optionally upstream from the GLP-1 receptor agonist coding sequence.
42. The method of paragraph 41, wherein the human insulin signal peptide comprises the sequence of SEQ ID NO: 13.
43. The method of any of paragraphs 29-42, wherein the transgene further comprises an intervening peptide 2 (IP2) peptide coding sequence downstream from the GLP-1 receptor agonist coding sequence.
44. The method of paragraph 43, wherein the IP2 peptide coding sequence comprises the sequence of SEQ ID NO: 8.
45. The method of any of paragraphs 29-44, wherein the transgene comprises the sequence of SEQ ID NO: 1.
46. The method of any of paragraphs 29-45, wherein the recombinant AAV genome further comprises a Woodchuck Hepatitis Virus Posttranscriptional Regulatory Element (WPRE) sequence, optionally downstream from the transgene and upstream from the polyA sequence.
47. The method of paragraph 46, wherein the WPRE sequence comprises the sequence of SEQ ID NO: 9.
48. The method of any of paragraphs 29-47, wherein the recombinant AAV genome comprises the sequence of SEQ ID NO: 2.
49. The method of any of paragraphs 29-47, wherein the recombinant AAV genome comprises the sequence of SEQ ID NO: 3.
50. The method of any of paragraphs 29-49, wherein the effective amount is about 5×10¹² to about 5×10¹⁴ AAV vector genomes (VG).
51. The method of any of paragraphs 29-50, wherein the injecting of the pancreatic tissue is via endoscopic ultrasound-guided fine needle injection (EUS-FNI).
52. The method of any of paragraphs 29-51, wherein the injecting of the pancreatic tissue comprises a single infusion.
53. The method of any of paragraphs 29-51, wherein the injecting of the pancreatic tissue comprises multiple infusions.
54. The method of paragraph 53, wherein the injecting of the pancreatic tissue comprises at least three infusions.
55. The method of any of paragraphs 29-54, wherein the pancreatic tissue is in a tail region of the pancreas.
56. The method of any of paragraphs 29-55, wherein the pancreatic tissue is in a body region of the pancreas.
57. A method for treating type 2 diabetes in a subject, comprising:
injecting pancreatic tissue of the subject with an effective amount of an adeno-associated virus (AAV) vector comprising (i) a recombinant AAV genome comprising a pancreatic islet cell specific promoter operably linked to a transgene comprising a GLP-1 receptor agonist coding sequence and (ii) AAV capsid proteins, optionally AAV9 capsid proteins, wherein the subject has type 2 diabetes.
58. The method of paragraph 57, wherein the subject has a BMI of at least 25 kg/m².
59. The method of paragraph 58, wherein the subject has a BMI of at least 30 kg/m².
60. The method of any of paragraphs 57-59, wherein the effective amount is sufficient to lower fasting blood glucose in the subject to below 126 mg/dL.
61. The method of any of paragraphs 57-59, wherein the effective amount is sufficient to lower fasting blood glucose in the subject to below 100 mg/dL.
62. The method of paragraph 61, wherein the effective amount is sufficient for the subject to maintain a BMI of less than 30 kg/m² for at least 14 days.
63. The method of paragraph 62, wherein the effective amount is sufficient for the subject to maintain a BMI of less than 30 kg/m² for at least 28 days.
64. The method of any of paragraphs 57-63, wherein the recombinant AAV genome comprises in the following 5′ to 3′ order: a first AAV inverted terminal repeat (ITR) sequence, the pancreatic islet cell specific promoter, the transgene, a polyadenylation (polyA) sequence, and a second
65. The method of any of paragraphs 64-67, wherein the GLP-1 receptor agonist coding sequence is a GLP-1₍₁₋₃₇₎ peptide coding sequence.
66. The method of paragraph 64 or 65, wherein: the first AAV inverted terminal repeat (ITR) sequence comprises the sequence of SEQ ID NO: 11, the pancreatic islet cell specific promoter comprises the sequence of SEQ ID NO: 6, the GLP-1 receptor agonist coding sequence of the transgene comprises the sequence of SEQ ID NO: 7, the WPRE comprises the sequence of SEQ ID NO: 9, the polyadenylation (polyA) sequence comprising the sequence of SEQ ID NO: 10, and the second AAV ITR comprising the sequence of SEQ ID NO: 12.
67. The method of any of paragraphs 57-66, wherein the pancreatic islet cell specific promoter comprises a human insulin promoter sequence.
68. The method of paragraph 67, wherein the human insulin promoter sequence comprises the sequence of SEQ ID NO: 6.
69. The method of any of paragraphs 57-68, wherein the transgene further comprises a human insulin signal peptide, optionally upstream from the GLP-1 receptor agonist coding sequence.
70. The method of paragraph 69, wherein the human insulin signal peptide comprises the sequence of SEQ ID NO: 13.
71. The method of any of paragraphs 57-70, wherein the transgene further comprises an intervening peptide 2 (IP2) peptide coding sequence downstream from the GLP-1 receptor agonist coding sequence.
72. The method of paragraph 71, wherein the IP2 peptide coding sequence comprises the sequence of SEQ ID NO: 8.
73. The method of any of paragraphs 57-72, wherein the transgene comprises the sequence of SEQ ID NO: 1.
74. The method of any of paragraphs 57-73, wherein the recombinant AAV genome further comprises a Woodchuck Hepatitis Virus Posttranscriptional Regulatory Element (WPRE) sequence, optionally downstream from the transgene and upstream from the polyA sequence.
75. The method of paragraph 74, wherein the WPRE sequence comprises the sequence of SEQ ID NO: 9.
76. The method of any of paragraphs 57-75, wherein the recombinant AAV genome comprises the sequence of SEQ ID NO: 2.
77. The method of any of paragraphs 57-75, wherein the recombinant AAV genome comprises the sequence of SEQ ID NO: 3.
78. The method of any of paragraphs 57-77, wherein the effective amount is about 5×10¹² to about 5×10¹⁴ AAV vector genomes (VG).
79. The method of any of paragraphs 57-78, wherein the injecting of the pancreatic tissue is via endoscopic ultrasound-guided fine needle injection (EUS-FNI).
80. The method of any of paragraphs 57-79, wherein the injecting of the pancreatic tissue comprises a single infusion.
81. The method of any of paragraphs 57-79, wherein the injecting of the pancreatic tissue comprises multiple infusions.
82. The method of paragraph 81, wherein the injecting of the pancreatic tissue comprises at least three infusions.
83. The method of any of paragraphs 57-82, wherein the pancreatic tissue is in a tail region of the pancreas.
84. The method of any of paragraphs 57-83, wherein the pancreatic tissue is in a body region of the pancreas.
85. A method for regulating blood glucose levels in a subject, comprising:

injecting pancreatic tissue of the subject with an effective amount of an adeno-associated virus (AAV) vector comprising (i) a recombinant AAV genome comprising a pancreatic islet cell specific promoter operably linked to a transgene comprising a GLP-1 receptor agonist coding sequence and (ii) AAV capsid proteins, optionally AAV9 capsid proteins, wherein the subject has a fasting blood glucose of at least 100 mg/dL.
86. The method of paragraph 85, wherein the subject has a fasting blood glucose of at least 126 mg/dL.
87. The method of paragraph 85 or 86, wherein the effective amount is sufficient to treat hyperglycemia in the subject.
88. The method of any of paragraphs 85-87, wherein the recombinant AAV genome comprises in the following 5′ to 3′ order: a first AAV inverted terminal repeat (ITR) sequence, the pancreatic islet cell specific promoter, the transgene, a polyadenylation (polyA) sequence, and a second
89. The method of any of paragraphs 85-88, wherein the GLP-1 receptor agonist coding sequence is a GLP-1 (1-37) peptide coding sequence.
90. The method of paragraph 88 or 89, wherein: the first AAV inverted terminal repeat (ITR) sequence comprises the sequence of SEQ ID NO: 11, the pancreatic islet cell specific promoter comprises the sequence of SEQ ID NO: 6, the GLP-1 receptor agonist coding sequence of the transgene comprises the sequence of SEQ ID NO: 7, the WPRE comprises the sequence of SEQ ID NO: 9, the polyadenylation (polyA) sequence comprising the sequence of SEQ ID NO: 10, and the second AAV ITR comprising the sequence of SEQ ID NO: 12.
91. The method of any of paragraphs 85-90, wherein the pancreatic islet cell specific promoter comprises a human insulin promoter sequence.
92. The method of paragraph 91, wherein the human insulin promoter sequence comprises the sequence of SEQ ID NO: 6.
93. The method of any of paragraphs 85-92, wherein the transgene further comprises a human insulin signal peptide, optionally upstream from the GLP-1 receptor agonist coding sequence.
94. The method of paragraph 93, wherein the human insulin signal peptide comprises the sequence of SEQ ID NO: 13.
95. The method of any of paragraphs 85-94, wherein the transgene further comprises an intervening peptide 2 (IP2) peptide coding sequence downstream from the GLP-1 receptor agonist coding sequence.
96. The method of paragraph 95, wherein the IP2 peptide coding sequence comprises the sequence of SEQ ID NO: 8.
97. The method of any of paragraphs 85-96, wherein the transgene comprises the sequence of SEQ ID NO: 1.
98. The method of any of paragraphs 85-97, wherein the recombinant AAV genome further comprises a Woodchuck Hepatitis Virus Posttranscriptional Regulatory Element (WPRE) sequence, optionally downstream from the transgene and upstream from the polyA sequence.
99. The method of paragraph 98, wherein the WPRE sequence comprises the sequence of SEQ ID NO: 9.
100. The method of any of paragraphs 85-99, wherein the recombinant AAV genome comprises the sequence of SEQ ID NO: 2.
101. The method of any of paragraphs 85-99, wherein the recombinant AAV genome comprises the sequence of SEQ ID NO: 3.
102. The method of any of paragraphs 85-101, wherein the effective amount is about 5×10¹² to about 5×10¹⁴ AAV vector genomes (VG).
103. The method of any of paragraphs 85-102, wherein the injecting of the pancreatic tissue is via endoscopic ultrasound-guided fine needle injection (EUS-FNI).
104. The method of any of paragraphs 85-103, wherein the injecting of the pancreatic tissue comprises a single infusion.
105. The method of any of paragraphs 85-104, wherein the injecting of the pancreatic tissue comprises multiple infusions.
106. The method of paragraph 105, wherein the injecting of the pancreatic tissue comprises at least three infusions.
107. The method of any of paragraphs 85-106, wherein the pancreatic tissue is in a tail region of the pancreas.
108. The method of any of paragraphs 85-107, wherein the pancreatic tissue is in a body region of the pancreas.
109. A polynucleotide comprising, in the 5′ to 3′ direction: a first AAV inverted terminal repeat (ITR) sequence comprising the sequence of SEQ ID NO: 11, a human insulin promoter comprising the sequence of SEQ ID NO: 6, a human insulin signal peptide coding sequence comprising the sequence of SEQ ID NO: 13, a GLP-1 coding sequence comprising the sequence of SEQ ID NO: 7, an IP2 coding sequence comprising the sequence of SEQ ID NO: 8, a WPRE sequence comprising the sequence of SEQ ID NO: 9, a polyA signal sequence comprising the sequence of SEQ ID NO: 10, and a second AAV ITR sequence comprising the sequence of SEQ ID NO: 12.
110. A polynucleotide comprising the nucleotide sequence of SEQ ID NO: 1.
111. A polynucleotide comprising the nucleotide sequence of SEQ ID NO: 2.
112. A polynucleotide comprising the nucleotide sequence of SEQ ID NO: 3.
113. An AAV viral vector comprising the polynucleotide of any of paragraphs 109-112 and AAV capsid proteins, optionally AAV9 capsid proteins.
114. An AAV viral vector comprising a recombinant AAV genome comprising the sequence of SEQ ID NO: 3 and AAV capsid proteins, optionally AAV9 capsid proteins.
115. A plasmid comprising the polynucleotide of any of paragraphs 109-112.
116. A plasmid comprising, in the 5′ to 3′ direction: a first AAV inverted terminal repeat (ITR) sequence, a human insulin promoter, a human insulin signal peptide coding sequence, a GLP-1 coding sequence, an IP2 coding sequence, a WPRE sequence, a polyA signal sequence, and a second AAV ITR sequence.
117. A plasmid comprising the sequence of SEQ ID NO: 4.
118. A system for weight loss and/or maintenance in a subject in need thereof, comprising:
(a) a gene therapy composition comprising an adeno-associated virus (AAV) vector comprising
(i) a recombinant AAV genome comprising a pancreatic islet cell specific promoter operably linked to a transgene comprising a GLP-1 receptor agonist coding sequence and an intervening peptide 2 (IP2) peptide coding sequence and (ii) AAV capsid proteins; and
(b) a depositing device for delivering an effective amount of the gene therapy composition to the subject, the depositing device comprising:
(i) an infusion assembly comprising a shaft, a lumen within the shaft, and a distal portion comprising a flexible needle with a diameter of no more than 27 G, wherein the shaft comprises a hyperechogenic coating and a stepped profile configured to prevent backflow of the gene therapy composition;
(ii) a syringe assembly constructed and arranged to provide the gene therapy composition to the infusion assembly;
(iii) a drive assembly constructed and arranged to operably attach to the syringe assembly and force the gene therapy composition from the syringe assembly, wherein the drive assembly comprises a housing, a constant force spring that applies a translating force to the translating assembly, a translating assembly, a cranking assembly configured to retract the translating assembly, and a locking assembly comprising a ratcheting lock and a sliding latch, wherein the locking assembly is configured to lock the translating assembly in a retracted position.
119. The system of paragraph 118, wherein the recombinant AAV genome comprises in the following 5′ to 3′ order: a first AAV inverted terminal repeat (ITR) sequence, the pancreatic islet cell specific promoter, the transgene, a polyadenylation (polyA) sequence, and a second AAV ITR.
120. The system of paragraph 118 or 119, wherein the GLP-1 receptor agonist coding sequence is a GLP-1 (1-37) peptide coding sequence.
121. The system of paragraph 119 or 120, wherein: the first AAV inverted terminal repeat (ITR) sequence comprises the sequence of SEQ ID NO: 11, the pancreatic islet cell specific promoter comprises the sequence of SEQ ID NO: 6, the GLP-1 receptor agonist coding sequence of the transgene comprises the sequence of SEQ ID NO: 7, the WPRE comprises the sequence of SEQ ID NO: 9, the polyadenylation (polyA) sequence comprising the sequence of SEQ ID NO: 10, and the second AAV ITR comprising the sequence of SEQ ID NO: 12.
122. The system of any of paragraphs 118-121, wherein the pancreatic islet cell specific promoter comprises a human insulin promoter sequence.
123. The system of paragraph 122, wherein the human insulin promoter sequence comprises the sequence of SEQ ID NO: 6.
124. The system of any of paragraphs 118-123, wherein the transgene further comprises a human insulin signal peptide, optionally upstream from the GLP-1 receptor agonist coding sequence.
125. The system of paragraph 124, wherein the human insulin signal peptide comprises the sequence of SEQ ID NO: 13.
126. The system of paragraph 125, wherein the IP2 peptide coding sequence is downstream from the GLP-1 receptor agonist coding sequence.
127. The system of paragraph 125 or 126, wherein the IP2 peptide coding sequence comprises the sequence of SEQ ID NO: 8.
128. The system of any of paragraphs 118-127, wherein the transgene comprises the sequence of SEQ ID NO: 1.
129. The system of any of paragraphs 118-128, wherein the recombinant AAV genome further comprises a Woodchuck Hepatitis Virus Posttranscriptional Regulatory Element (WPRE) sequence, optionally downstream from the transgene and upstream from the polyA sequence.
130. The system of paragraph 129, wherein the WPRE sequence comprises the sequence of SEQ ID NO: 9.
131. The system of any of paragraphs 118-130, wherein the recombinant AAV genome comprises the sequence of SEQ ID NO: 2.
132. The system of any of paragraphs 118-130, wherein the recombinant AAV genome comprises the sequence of SEQ ID NO: 3.
133. The system of any of paragraphs 118-132, wherein the effective amount is about 5×10¹² to about 5×10¹⁴ AAV vector genomes (VG).
134. A method for weight loss and/or maintenance in a subject in need thereof, comprising:
(a) providing the system of any of paragraphs 118-133;
(b) advancing the infusion assembly to a deposit site in the pancreas of the subject;
(c) inserting the needle of the infusion assembly into parenchymal tissue of the pancreas and
(d) delivering the gene therapy composition at a controlled flow rate of no more than 20 mL/min into the deposit site via the infusion assembly.
135. The method of paragraph 134, comprising delivering the gene therapy a single time.
136. The method of paragraph 134, comprising delivering the gene therapy multiple times.
137. The method of paragraph 136, comprising delivering the gene therapy at least three times.
138. A system for treating a subject, comprising:
a gene therapy composition comprising an adeno-associated virus (AAV) vector comprising (i) a recombinant AAV genome comprising a pancreatic islet cell specific promoter operably linked to a transgene comprising a GLP-1 receptor agonist coding sequence and an intervening peptide 2 (IP2) peptide coding sequence and (ii) AAV capsid proteins; and an infusion system configured to deliver an effective amount of the gene therapy composition to the subject in one or more injections, the infusion system comprising a drive mechanism configured to control dispensing of the gene therapy composition.
139. The system of paragraph 138, wherein the infusion system is configured to deliver the effective amount of the gene therapy composition in multiple injections, including a first amount of the gene therapy composition in response to a first activation of the drive mechanism and to control dispensing of a second amount of the gene therapy composition in response to a second activation of the drive mechanism after the first activation.
140. The system of paragraph 138 or 139, wherein the infusion system further comprises a body, a lumen extending from the body, and a needle coupled to the lumen and having an adjustable exposed length beyond a sheath, wherein the infusion system is configured to deliver the effective amount of the gene therapy composition to the subject via the lumen and needle.
141. The system of any of paragraphs 138-140, wherein the infusion system further comprises an adjustment assembly configured to set the adjustable exposed length of the needle beyond the sheath, the adjustment assembly comprising a movable depth adjustment mechanism coupled to a shaft of the infusion system.
142. The system of paragraph 141, wherein the movable depth adjustment mechanism comprises a movable clip.
143. The system of any of paragraphs 138-142, wherein the needle comprises a conical tip and an opening displaced from the conical tip.
144. The system of paragraph 143, wherein the needle is a 27 gauge needle and comprises a coating.
145. The system of any of paragraphs 138-144, wherein the drive mechanism comprises a constant force spring, a drive pin, and a stop pin, and wherein in response to the first activation the constant force spring is configured to move the drive pin a first distance until being stopped by the stop pin.
146. The system of paragraph 145, wherein the lumen comprise a material configured to prevent adsorption of the gene therapy composition.
147. The system of paragraph 145 or 146, wherein the lumen comprises nylon.
148. A system for treating a subject, comprising:
a gene therapy composition comprising an adeno-associated virus (AAV) vector comprising (i) a recombinant AAV genome comprising a pancreatic islet cell specific promoter operably linked to a transgene comprising a GLP-1 receptor agonist coding sequence and an intervening peptide 2 (IP2) peptide coding sequence and (ii) AAV capsid proteins; and
an infusion system for delivering an effective amount of the gene therapy composition to the subject, the infusion system comprising:
a body configured to hold a syringe containing the gene therapy composition;
a lumen coupled to the body and configured to transport the gene therapy composition; and
a 27 gauge needle coupled to the lumen and having a conical tip and a port displaced from the conical tip.
149. The system of paragraph 148, wherein the needle further comprises a hyperechogenic coating.
150. The system of paragraph 148 or 149, further comprising a sheath surround the lumen, wherein the infusion system is configured to adjust a length by which the 27 gauge needle extends from the sheath.
151. The system of any of paragraphs 148-150, further comprising a fluid stop mechanism operable to stop flow of the gene therapy composition in the lumen.
152. The system of paragraph 151, wherein the fluid stop mechanism is operable by hand.
153. The system of paragraph 152, wherein the fluid stop mechanism is disposed between the body and the 27 gauge needle.
154. A system for treating a subject, comprising:
a gene therapy composition comprising an adeno-associated virus (AAV) vector comprising (i) a recombinant AAV genome comprising a pancreatic islet cell specific promoter operably linked to a transgene comprising a GLP-1 receptor agonist coding sequence and an intervening peptide 2 (IP2) peptide coding sequence and (ii) AAV capsid proteins; and
an infusion system for delivering an effective amount of the gene therapy composition to the subject, the infusion system comprising:
a body portion having a receptacle;
a shaft coupled to the body portion and having a lumen disposed therein;
a needle positioned at a distal end of the shaft;
a syringe disposed in the receptacle of the body portion and arranged to provide the gene therapy composition to the needle via the lumen within the shaft;
a drive mechanism arranged to contact a plunger of the syringe and force the gene therapy composition from the syringe; and
an adjustment mechanism arranged to adjust an insertion depth of the needle by adjusting a length along which the needle is movable relative to the lumen, the adjustment mechanism comprising a slider and a clip coupled to the slider, wherein adjusting a position of the clip along the slider adjusts a length along which the body portion is movable relative to the clip thereby adjusting the insertion depth of the needle.
155. The system of paragraph 154, wherein the length along which the body portion is movable relative to the clip is adjustable between 0 cm and 6 cm.
156. The system of paragraph 154 or 155, wherein the lumen comprise a material configured to prevent adsorption of the gene therapy composition.
157. The system of paragraph 154 or 155, wherein the lumen comprises nylon.
158. A system for treating a subject, comprising:
a gene therapy composition comprising an adeno-associated virus (AAV) vector comprising (i) a recombinant AAV genome comprising a pancreatic islet cell specific promoter operably linked to a transgene comprising a GLP-1 receptor agonist coding sequence and an intervening peptide 2 (IP2) peptide coding sequence and (ii) AAV capsid proteins; and
an infusion system for delivering an effective amount of the gene therapy composition to the subject, the infusion system comprising:
a body portion having a receptacle;
a shaft coupled to the body portion and having a lumen disposed therein;
a needle positioned at a distal end of the shaft;
a syringe disposed in the receptacle of the body portion and arranged to provide the gene therapy composition to the needle via the lumen within the shaft;
a drive mechanism arranged to contact a plunger of the syringe and force the gene therapy composition from the syringe; and
a fluid stop mechanism operable to stop flow of the gene therapy composition in the lumen.
159. The system of paragraph 158, wherein the fluid stop mechanism is operable by hand.
160. The system of paragraph 158 or 159, wherein the fluid stop mechanism comprises a slidable block oriented substantially perpendicularly to the lumen.
161. The system of any of paragraphs 158-160, wherein the lumen comprise a material configured to prevent adsorption of the gene therapy composition.
162. The system of any of paragraphs 158-160, wherein the lumen comprises nylon.
163. An endoscopic ultrasound (EUS)-guided infusion system for delivering a treatment material into a target tissue of a subject, comprising: (a) an infusion device comprising: a handle configured to couple to an endoscope; an outer sheath configured to extend through a working channel of the endoscope; an inner fluid lumen assembly disposed within the outer sheath and movable relative to the outer sheath, the inner fluid lumen assembly defining a fluid pathway and including an infusion needle at a distal end; a pressure-driven infusion mechanism on the handle configured to drive the treatment material through the fluid pathway; and a fluid stop mechanism positioned along the fluid pathway between the pressure-driven infusion mechanism and the infusion needle; and (b) an electronic control subsystem comprising an actuator operatively coupled to the fluid stop mechanism and a controller in communication with an ultrasound imaging system, wherein the controller is configured to receive EUS images from the ultrasound imaging system, compute at least one dispersion metric indicative of distribution of the treatment material within the target tissue, and gate delivery of the treatment material by commanding the actuator to transition the fluid stop mechanism between an open state permitting flow through the fluid pathway and an occluded state inhibiting flow through the fluid pathway based at least in part on the at least one dispersion metric.
164. The system of paragraph 163, wherein the fluid stop mechanism comprises a clamping element configured to compress a flexible segment of the fluid pathway, and wherein the actuator comprises at least one of a micro-motor, stepper motor, solenoid, shape memory alloy actuator, or piezoelectric clamp configured to reposition the clamping element.
165. The system of paragraph 163, wherein the fluid stop mechanism is operable in multiple modes including: (i) an emergency stop mode in which the fluid pathway is substantially fully occluded; (ii) an open mode; and (iii) at least one regulated-flow mode in which the fluid pathway is partially restricted to define a selected effective orifice area such that the fluid stop mechanism operates as an adjustable flow regulator.
166. The system of paragraph 165, further comprising one or more sensors selected from a pressure sensor upstream of the fluid stop mechanism, a pressure sensor downstream of the fluid stop mechanism, a flow sensor, and an actuator position encoder, wherein the controller is configured to select the selected effective orifice area based on at least one of a measured pressure, a measured flow rate, an actuator position feedback signal, a measured temperature, a measured viscosity proxy, or a user-entered identifier of the treatment material.
167. The system of paragraph 165, wherein the controller is configured to initiate infusion in a soft-start mode by commanding the fluid stop mechanism to move from a more restrictive regulated-flow mode toward a less restrictive regulated-flow mode over a ramp interval, and to revert from any regulated-flow mode to the emergency stop mode upon receiving an emergency stop command.
168. The system of paragraph 163, further comprising a flow sensor coupled to the fluid pathway, wherein the controller is configured to verify a nominal flow start profile at an onset of infusion, detect off-nominal flow indicative of at least one of occlusion, partial occlusion, leak, disconnection, bubble/air passage, or excessive backpressure, and, responsive thereto, automatically command occlusion using the fluid stop mechanism and/or present a prompt instructing an operator to pause and reposition the infusion needle.
169. The system of paragraph 168, wherein the flow sensor comprises a non-contact optical or infrared sensor located in a reusable module and configured to sense flow through a transparent tubing segment of the fluid pathway.
170. The system of paragraph 163, further comprising a needle force sensor mechanically coupled to the infusion needle and configured to generate a signal indicative of at least one of axial loading, lateral loading, or transient loading events, wherein the controller is configured to compare the signal to a plurality of thresholds and, when a threshold is exceeded, provide at least one of an operator warning, audible feedback, visual feedback, haptic feedback, automatic infusion pausing via the fluid stop mechanism, or a retraction cue.
171. The system of paragraph 170, wherein the needle force sensor comprises at least one of a strain gauge, thin-film piezoresistive element, capacitive strain sensor, optical strain sensor, or impedance-based strain sensing trace disposed in a hub region proximal to a needle shaft and adjacent a transition region between a rigid tube and a flexible lumen segment of the inner fluid lumen assembly.
172. The system of paragraph 163, wherein the controller implements an arming state machine comprising: (i) a landmark-confirmation state requiring confirmation of one or more anatomic landmarks in the EUS images and a stability dwell time; (ii) a needle-extension-enabled state; (iii) an infusion-enabled state; and (iv) a stop/retract state that enforces needle retraction and/or operator repositioning.
173. The system of paragraph 172, further comprising one or more position sensors coupled to at least one depth-setting structure selected from a stop, slider, sheath adjustment mechanism, or carriage, wherein the controller is configured to determine an expected distal needle extension beyond the outer sheath based on a signal from the one or more position sensors and render a virtual needle overlay representing the expected needle extension relative to an ultrasound image.
174. The system of paragraph 173, further comprising an electromagnetic tracking sensor or magnetic marker associated with the infusion needle, wherein the controller is configured to combine tracking information with the signal from the one or more position sensors to update the virtual needle overlay and generate an alarm or lockout when a projected needle path intersects a keep-out region.
175. The system of paragraph 173, wherein the controller is configured to compute, from the EUS images or an EUS-derived three-dimensional reconstruction, a virtual fixture defining a keep-in corridor and one or more keep-out zones, and to prevent at least one of needle advancement, needle extension beyond the sheath, or infusion arming when the virtual needle overlay leaves the keep-in corridor or enters a keep-out zone.
176. The system of paragraph 163, wherein the infusion device further comprises a motorized needle advancement drive configured to advance and retract the inner fluid lumen assembly relative to the outer sheath, and wherein the controller is configured to perform programmed insertion to a planned extension derived from at least one of depth-setting structure settings or EUS-derived depth estimates, and to retract the infusion needle upon detection of a safety condition or a user command.
177. The system of paragraph 163, further comprising a dual-fluid delivery assembly comprising a first fluid reservoir, a second fluid reservoir, a first fluid path, a second fluid path, and at least one flow control element selected from a valve manifold, multi-port selector, or first and second independent actuators, wherein the controller is configured to deliver a verification bolus from the first fluid reservoir and, thereafter, deliver a therapeutic infusion from the second fluid reservoir through the infusion needle.
178. The system of paragraph 177, wherein the verification bolus comprises microbubble contrast and the controller is configured to initiate contrast harmonic imaging to confirm at least one of needle tip localization, outflow, or early dispersion behavior prior to delivering the therapeutic infusion, and optionally to compensate for dead volume by delivering a predetermined priming volume before interpreting a contrast-enhanced signal.
179. The system of paragraph 163, wherein the infusion needle comprises an active beacon configured, upon command, to generate a distinct ultrasound signature that is separable from a passive echogenic coating, the active beacon comprising a piezoelectric element mechanically coupled to a portion of the infusion needle or a structural member coupled to the infusion needle.
180. The system of paragraph 163, wherein the controller is configured to reduce backflow by controlling a pressure and/or flow profile including: a ramped increase at initiation of infusion; a post-infusion dwell period during which pressure decays while the infusion needle remains positioned in tissue; and a staged withdrawal sequence coordinated with partial restriction or occlusion of the fluid stop mechanism.
181. The system of paragraph 163, wherein the outer sheath and the inner fluid lumen assembly include friction reduction features configured to reduce sliding friction between an outer diameter of the inner fluid lumen assembly and an inner diameter of the outer sheath, the friction reduction features comprising at least one of: a lubricious coating or surface treatment on at least one of the mating surfaces; a sleeve or jacket disposed over a laser-cut outer tube of the inner fluid lumen assembly; interior patterning of the outer sheath; or a radial clearance between the outer diameter and the inner diameter in the range of approximately 0.00075 inches to approximately 0.0135 inches.
182. The system of paragraph 163, wherein the electronic control subsystem is provided in a reusable attachment module configured to couple to the handle while fluid-contacting components remain disposable, and wherein the system is configured for fail-safe behavior in which the fluid stop mechanism defaults to the occluded state upon loss of power.
183. An endoscopic ultrasound (EUS)-guided infusion system for delivering a therapeutic composition into target tissue of a subject, comprising: (a) a handheld infusion device configured to couple to an endoscope, the handheld infusion device comprising (i) an outer sheath configured to extend through a working channel of the endoscope, (ii) an inner fluid lumen assembly disposed within and movable relative to the outer sheath, the inner fluid lumen assembly including an infusion needle at a distal end, (iii) a pressure-driven infusion mechanism configured to drive the therapeutic composition through a fluid pathway to the infusion needle, and (iv) a fluid stop mechanism positioned along the fluid pathway; and (b) a controller in communication with an ultrasound imaging system and operatively coupled to an actuator that actuates the fluid stop mechanism, wherein the controller is configured to determine, from EUS imaging, a dispersion condition indicative of distribution of the therapeutic composition in the target tissue, and to gate delivery by commanding the actuator to transition the fluid stop mechanism between a flow-permitting state and a flow-inhibiting state based at least in part on the dispersion condition.
184. The system of paragraph 183, wherein the dispersion condition comprises at least one of an extent of spread, a rate of spread, a boundary proximity metric, or a contrast-enhanced dispersion metric derived from contrast harmonic imaging.
185. The system of paragraph 183, wherein the controller is configured to execute a delivery state machine comprising at least an arming state, an infusion-enabled state, and a pause or stop state, and to require satisfaction of one or more imaging-derived criteria to transition from the arming state to the infusion-enabled state.
186. An endoscopic infusion device configured for delivery of a therapeutic composition through an endoscope working channel, comprising: (a) a handle configured to receive a medicament container and comprising a pressure-driven infusion mechanism configured to apply pressure to the medicament container to drive the therapeutic composition; (b) an outer sheath configured to extend through the working channel; (c) an inner fluid lumen assembly disposed within the outer sheath and including an infusion needle, the inner fluid lumen assembly defining a fluid pathway from the medicament container to the infusion needle; and (d) a fluid stop mechanism positioned along the fluid pathway, the fluid stop mechanism comprising a controllable restriction element configured to operate in (i) a fully open condition, (ii) a fully occluded condition, and (iii) at least one intermediate restriction condition that reduces a volumetric flow rate for a given pressure produced by the pressure-driven infusion mechanism, whereby the fluid stop mechanism functions as a flow regulator to accommodate therapeutic compositions having different viscosities while using the same pressure-driven infusion mechanism.
187. The device of paragraph 186, wherein the controllable restriction element comprises a clamp configured to compress a flexible segment of the fluid pathway and to define an effective orifice area that is selectable among multiple discrete settings or continuously variable settings.
188. The device of paragraph 186, further comprising a controller configured to select the intermediate restriction condition based on at least one of a measured pressure, a measured flow rate, an actuator position, a measured temperature, a viscosity proxy, or an identifier of the therapeutic composition.
189. An endoscopic infusion system for delivering a therapeutic composition into target tissue, comprising: (a) an infusion device comprising a handle, a sheath configured to extend through an endoscope working channel, an inner fluid lumen assembly with an infusion needle, and a fluid pathway from a medicament container to the infusion needle; (b) a flow sensor configured to sense a flow characteristic of the therapeutic composition in the fluid pathway; and (c) a controller configured to detect, using the flow characteristic, an off-nominal delivery condition and to output at least one corrective action comprising automatically inhibiting flow through the fluid pathway, prompting an operator to pause and reposition the infusion needle, or both.
190. The system of paragraph 189, wherein the flow sensor comprises a non-contact optical or infrared sensor disposed in a reusable module and configured to sense flow through a transparent tubing segment coupled to the infusion device.
191. The system of paragraph 189, wherein the off-nominal delivery condition comprises at least one of failed-flow, partial occlusion, leakage, disconnection, air or bubble passage, or excessive backpressure indicative of bolus buildup risk.
192. An endoscopic infusion system for delivering a therapeutic composition through an endoscope working channel, comprising: (a) an infusion device comprising an outer sheath, an inner fluid lumen assembly movable relative to the outer sheath and including an infusion needle, and a handle configured to advance the inner fluid lumen assembly and deliver the therapeutic composition; and (b) a force sensing subsystem mechanically coupled to the infusion needle and configured to generate a signal indicative of at least one of axial load or lateral load applied to the infusion needle, wherein the system is configured to compare the signal to at least one threshold and, responsive to exceeding the threshold, provide at least one of a user warning, haptic feedback, automatic pausing of infusion, or an instruction to retract or reposition the infusion needle.
193. The system of paragraph 192, wherein the force sensing subsystem is configured to detect lateral loading and to output a graded indication corresponding to a green zone, a warning zone, and a stop zone.
194. The system of paragraph 192, wherein the force sensing subsystem comprises at least one strain-based sensor disposed in a hub region proximal to a needle shaft such that bending of the needle shaft is transduced into the signal.
195. An EUS-guided infusion system for delivering a therapeutic composition into target tissue, comprising: (a) an infusion device comprising a sheath configured to extend through an endoscope working channel, an inner fluid lumen assembly including an infusion needle and movable relative to the sheath, and at least one depth-setting structure configured to set or constrain an extension of the infusion needle beyond the sheath; (b) at least one sensor coupled to the depth-setting structure and configured to generate a signal indicative of a device state from which an expected needle extension is determinable; and (c) a controller configured to (i) generate, based on the signal, a virtual needle representation indicative of the expected needle extension, and (ii) provide at least one safety output comprising an overlay on an ultrasound display, an alarm, or a lockout that prevents at least one of needle extension or infusion arming when a projected needle path violates a safety condition.
196. The system of paragraph 195, further comprising an electromagnetic tracking element associated with the infusion device, wherein the controller is configured to combine tracking information with the signal indicative of the device state to update the virtual needle representation.
197. The system of paragraph 195, wherein the safety condition comprises intersection with a keep-out region or departure from a keep-in corridor defined from ultrasound imaging or an ultrasound-derived reconstruction.
198. An endoscopic infusion system comprising a staged dual-fluid delivery assembly, the system comprising: (a) an infusion device including a sheath configured to extend through an endoscope working channel, an inner fluid lumen assembly with an infusion needle, and a distal delivery lumen fluidly coupled to the infusion needle; (b) a first fluid reservoir containing a verification fluid and a second fluid reservoir containing a therapeutic composition; and (c) a flow control subsystem configured to deliver, through the infusion needle, (i) a verification bolus from the first fluid reservoir followed by (ii) a therapeutic delivery from the second fluid reservoir, wherein the flow control subsystem comprises at least one of a valve manifold configured to selectively connect the distal delivery lumen to the first fluid reservoir or the second fluid reservoir, or first and second independent actuators configured to drive fluid from the first and second fluid reservoirs, respectively.
199. The system of paragraph 198, wherein the verification fluid comprises microbubble contrast and the system is configured to use contrast-enhanced ultrasound imaging to confirm needle tip localization and/or early dispersion prior to the therapeutic delivery.
200. The system of paragraph 198, wherein the system is configured to compensate for dead volume by delivering a predetermined priming volume before interpreting verification feedback or before initiating the therapeutic delivery.
201. An endoscopic infusion device configured for ultrasound-guided placement and infusion, comprising: (a) a sheath configured to extend through an endoscope working channel; (b) an inner fluid lumen assembly disposed within the sheath and including an infusion needle; and (c) an active ultrasound visibility element mechanically coupled to the infusion needle and configured, upon command, to generate a distinct acoustic signature detectable in ultrasound imaging to facilitate localization of the infusion needle.
202. The device of paragraph 201, wherein the active ultrasound visibility element comprises a piezoelectric actuator configured to vibrate at or near an ultrasound imaging frequency or a harmonic or subharmonic thereof.
203. The device of paragraph 201, wherein the infusion needle has a gauge of 27 G or smaller and the active ultrasound visibility element is configured to facilitate visualization at the smaller gauge.
204. A method of reducing backflow during ultrasound-guided infusion of a therapeutic composition into target tissue using an endoscopic infusion device having a sheath and an infusion needle, the method comprising: (a) advancing the infusion needle into the target tissue; (b) delivering the therapeutic composition according to a controlled pressure or flow profile that includes a ramped increase at initiation of infusion; (c) after delivering a target volume, maintaining the infusion needle in the target tissue for a dwell period while allowing pressure to decay in a controlled manner; and (d) retracting the infusion needle according to a staged withdrawal sequence coordinated with partial restriction or occlusion of a fluid pathway to reduce expression of the therapeutic composition along a backflow pathway.
204. The method of paragraph 204, wherein coordinating comprises actuating a fluid stop mechanism from a flow-permitting condition to a partially restricted condition during at least a portion of the dwell period or staged withdrawal sequence.
205. The method of paragraph 204, further comprising selecting at least one parameter of the ramped increase, dwell period, or staged withdrawal sequence based on at least one of measured pressure, measured flow, or an inferred tissue compliance metric.
206. An endoscopic infusion device configured to be advanced through an endoscope working channel, comprising: (a) an outer sheath having an inner diameter; and (b) an inner fluid lumen assembly disposed within the outer sheath, the inner fluid lumen assembly having an outer diameter and being configured to translate relative to the outer sheath to extend and retract an infusion needle, wherein the outer sheath and the inner fluid lumen assembly include friction-reduction features configured to reduce sliding friction between the outer diameter and the inner diameter while maintaining lateral support sufficient to resist buckling of the inner fluid lumen assembly during puncture.
207. The device of paragraph 207, wherein the friction-reduction features comprise at least one of a lubricious coating, a silicone-based coating, a dry-film lubricant, a hydrophobic coating, a hydrophilic coating, a PTFE-based coating, or plasma-treated surface on at least one of the outer diameter or the inner diameter.
208. The device of paragraph 207, wherein the inner diameter has an interior pattern configured to reduce contact area, the interior pattern comprising at least one of micro-ridges, spiral ridges, dimpled regions, ribbing, or a tri-lobed profile.
209. An endoscopic infusion system comprising a motorized needle deployment subsystem, the system comprising: (a) an infusion device comprising a sheath configured to extend through an endoscope working channel and an inner fluid lumen assembly movable relative to the sheath and including an infusion needle; (b) a motorized drive coupled to the inner fluid lumen assembly and configured to advance and retract the infusion needle relative to the sheath; and (c) a controller configured to perform a programmed insertion sequence by commanding the motorized drive to advance the infusion needle toward a planned extension and to inhibit further advancement and/or retract the infusion needle upon detection of a safety condition.
210. The system of paragraph 210, wherein the safety condition is determined based on at least one of a force signal, a flow signal, a pressure signal, or an imaging-derived criterion from ultrasound imaging.
211. The system of paragraph 210, wherein the programmed insertion sequence includes an intermediate pause for verification prior to enabling infusion, and the controller is configured to prevent infusion arming until the verification is satisfied.
212. The system of paragraph 183, wherein the controller is configured to command the fluid stop mechanism to an intermediate restriction condition to modulate a dispersion rate and/or to limit a maximum flow rate while maintaining the dispersion condition within a target range.
213. The system of paragraph 183, wherein the controller is configured to provide, on an ultrasound display, a visual indication of an armed state and at least one value corresponding to the dispersion condition, and to require a user confirmation input to resume delivery after a pause triggered by the dispersion condition.
214. The device of paragraph 186, wherein the pressure-driven infusion mechanism comprises at least one constant-force spring, pneumatic pressure source, or motorized syringe drive configured to generate a target pressure range at a medicament container.
215. The system of paragraph 189, further comprising a fluid stop mechanism and an actuator configured to automatically occlude the fluid pathway responsive to detection of the off-nominal delivery condition.
216. The system of paragraph 192, wherein the system is configured to automatically retract the infusion needle into the sheath when lateral load exceeds a withdrawal threshold.
217. The system of paragraph 195, wherein the at least one sensor coupled to the depth-setting structure comprises at least one of a Hall sensor and magnet, an optical encoder, a potentiometer, a resistive track, or a limit switch integrated into the handle.
218. The system of paragraph 198, further comprising one or more check valves configured to inhibit cross-contamination and unintended mixing between the verification fluid and the therapeutic composition.
219. The device of paragraph 201, wherein the active ultrasound visibility element is configured for intermittent activation in response to a user input on the handle, a foot pedal input, or a controller command, to facilitate reacquisition of the infusion needle while reducing imaging artifacts.
220. The system of paragraph 210, wherein the motorized drive and controller are configured to permit manual override of needle advancement by an operator.
221. The system of paragraph 210, wherein the motorized drive comprises a clutch, decoupler, or backdrivable transmission configured to allow the operator to retract the infusion needle independent of motor power.
223. The system of paragraph 183, wherein the controller and actuator are provided in a reusable attachment module configured to couple to the handle while fluid-contacting components remain disposable, and wherein the system is configured such that the fluid stop mechanism defaults to a flow-inhibiting state upon loss of power.
224. A recombinant adeno-associated virus (rAAV) vector, comprising: (a) an rAAV vector genome sequence that is at least 80% identical to the nucleotide sequence of SEQ ID NO: 3; and (b) AAV capsid proteins.
225. The rAAV vector of paragraph 224, wherein the rAAV vector genome sequence comprises an open reading frame encoding glucagon-like peptide-1 (GLP-1), optionally GLP-1 (1-37).
226. The rAAV vector of paragraph 224 or 225, wherein the open reading frame further encodes intervening peptide 2 (IP2).
227. The rAAV vector of any one of paragraphs 224-226, wherein the open reading frame further encodes a human insulin signal peptide.
228. The rAAV vector of any one of paragraphs 225-227, wherein the open reading frame is operably linked to a pancreatic beta cell-specific promoter.
229. The rAAV vector of paragraph 228, wherein the pancreatic beta cell-specific promoter comprises three tandem repeats of a human insulin core promoter.
230. The rAAV vector of any one of paragraphs 224-227, wherein the rAAV genome sequence is at least 90% identical to the nucleotide sequence of SEQ ID NO: 3.
231. The rAAV vector of paragraph 230, wherein the rAAV genome sequence is at least 95% identical to the nucleotide sequence of SEQ ID NO: 3.
232. The rAAV vector of paragraph 231, wherein the rAAV genome sequence comprises the nucleotide sequence of SEQ ID NO: 3.
233. The rAAV vector of any one of paragraphs 224-232, wherein the rAAV vector genome sequence comprises AAV2 inverted terminal repeats (ITRs).
234. The rAAV vector of any one of paragraphs 224-233, wherein the AAV capsid proteins comprise AAV9 capsid proteins.
235. A gene therapy composition comprising the rAAV vector of any one of paragraphs 224-234 and at least one excipient.
236. The gene therapy composition of paragraph 235, wherein at least one excipient of the composition is selected from phosphate buffered saline and Poloxamer 188, optionally 0.001% Poloxamer 188.
237. The gene therapy composition of paragraph 235 or 236, wherein the composition has a pH value of about 6.5 to about 7.5, optionally about 7.2.
238. A method of treating Type 2 diabetes in a human subject in need thereof, comprising: administering, by endoscopic ultrasound-guided intraparenchymal infusion (EUS-IPI), a therapeutically effective amount of the gene therapy composition of any one of paragraphs 235-237 to pancreatic tissue of the human subject.
239. The method of paragraph 238, wherein the EUS-IPI is performed using a delivery device comprising:
(a) an infusion needle having a gauge of 27 G, a conical tip, and a side opening proximal to the conical tip, wherein the infusion needle comprises an echogenic coating on at least a portion of its outer surface;
(b) a sheath surrounding the infusion needle, wherein the sheath is adjustable in length to accommodate an echoendoscope having a working channel diameter of at least 2.5 mm, optionally at least 2.8 mm; and
(c) a handle attached to the sheath and having an integrated syringe pump, wherein administering the therapeutically effective amount of the gene therapy composition comprises delivering the gene therapy composition through the infusion needle at a controlled flow rate and pressure by actuating the syringe pump.
240. The method of paragraph 238 or 239, wherein the infusion is at a controlled pressure and rate to avoid pancreatic duct injury.
241. The method of paragraph 238 and 239, wherein the therapeutically effective amount is a single dose of about 3.0×10¹³ vector genomes (VG) to about 5.0×10¹³ VG, preferably 4.0×10¹³ VG.
242. The method of paragraph 238 and 239, wherein the therapeutically effective amount is a single dose of about 1.0×10¹⁴ VG to about 2.0×10¹⁴ VG, preferably 1.2×10¹⁴ VG.
243. The method of paragraph 238 and 239, wherein the therapeutically effective amount is a single dose of about 3.0×10¹⁴ VG to about 4.0×10¹⁴ VG, preferably 3.6×10¹⁴ VG.
244. The method of any one of paragraphs 238-243, wherein the therapeutically effective amount is administered via two injections into the body and/or tail of the pancreas, optionally two 1 mL injections.

### EQUIVALENTS AND SCOPE

The indefinite articles “a” and “an,” as used herein in the specification and in the claims, unless clearly indicated to the contrary, should be understood to mean “at least one.”

The phrase “and/or,” as used herein, should be understood to mean “either or both” of the elements so conjoined, i.e., elements that are conjunctively present in some cases and disjunctively present in other cases. Multiple elements listed with “and/or” should be construed as “one or more” of the elements so conjoined. Other elements may optionally be present other than the elements specifically identified by the “and/or” clause, whether related or unrelated to those elements specifically identified.

As used herein, the phrase “at least one,” in reference to a list of one or more elements, should be understood to mean at least one element selected from any one or more of the elements in the list, but not necessarily including at least one of each and every element specifically listed, and not excluding any combinations of elements in the list. This definition also allows that elements may optionally be present other than those specifically identified within the list to which the phrase “at least one” refers, whether related or unrelated to those elements specifically identified.

It should also be understood that, unless clearly indicated to the contrary, in any methods claimed herein that include more than one step or act, the order of the steps or acts is not necessarily limited to the order in which they are recited.

Use of ordinal terms such as “first,” “second,” “third,” et cetera, does not by itself connote any priority, precedence, or order of one element over another, or the temporal order in which acts of a method are performed. Such terms are used merely as labels to distinguish one element having a certain name from another having the same name.

In the claims, as well as in the specification above, all transitional phrases such as “comprising,” “including,” “carrying,” “having,” “containing,” “involving,” “holding,” and “composed of” are to be understood as open-ended, i.e., meaning including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value. Where a range of values is provided, each value between and including the upper and lower ends of the range is specifically contemplated and described herein.

All references, patents, and patent applications disclosed herein are incorporated by reference with respect to the subject matter for which each is cited, which in some cases may encompass the entirety of the document.

Having described above several aspects of at least one embodiment, it is to be appreciated that various alterations, modifications, and improvements will readily occur to those skilled in the art. Such alterations, modifications, and improvements are intended to be the object of this disclosure. Accordingly, the foregoing description and drawings are by way of example only.

What is claimed is:

1. A recombinant adeno-associated virus (rAAV) vector, comprising:
(a) an rAAV vector genome sequence that is at least 80% identical to the nucleotide sequence of SEQ ID NO: 3; and
(b) AAV capsid proteins.

2. The rAAV vector of claim 1, wherein the rAAV vector genome sequence comprises an open reading frame encoding glucagon-like peptide-1 (GLP-1), optionally GLP-1 (1-37).

3. The rAAV vector of claim 1, wherein the open reading frame further encodes intervening peptide 2 (IP2).

4. The rAAV vector of claim 1, wherein the open reading frame further encodes a human insulin signal peptide.

5. The rAAV vector of claim 2, wherein the open reading frame is operably linked to a pancreatic beta cell-specific promoter.

6. The rAAV vector of claim 5, wherein the pancreatic beta cell-specific promoter comprises three tandem repeats of a human insulin core promoter.

7. The rAAV vector of claim 1, wherein the rAAV genome sequence is at least 90% identical to the nucleotide sequence of SEQ ID NO: 3.

8. The rAAV vector of claim 7, wherein the rAAV genome sequence is at least 95% identical to the nucleotide sequence of SEQ ID NO: 3.

9. The rAAV vector of claim 8, wherein the rAAV genome sequence comprises the nucleotide sequence of SEQ ID NO: 3.

10. The rAAV vector of claim 1, wherein the rAAV vector genome sequence comprises AAV2 inverted terminal repeats (ITRs).

11. The rAAV vector of claim 1, wherein the AAV capsid proteins comprise AAV9 capsid proteins.

12. A gene therapy composition: comprising the rAAV vector of claim 1 and at least one excipient.

13. The gene therapy composition of claim 12, wherein at least one excipient of the composition is selected from phosphate buffered saline and Poloxamer 188, optionally 0.001% Poloxamer 188.

14. The gene therapy composition of claim 12, wherein the composition has a pH value of about 6.5 to about 7.5, optionally about 7.2.

15. A method of treating Type 2 diabetes in a human subject in need thereof, comprising:
administering, by endoscopic ultrasound-guided intraparenchymal infusion (EUS-IPI), a therapeutically effective amount of the gene therapy composition of claim 12 to pancreatic tissue of the human subject.

16. The method of claim 15, wherein the EUS-IPI is performed using a delivery device comprising:
(a) an infusion needle having a gauge of 27 G, a conical tip, and a side opening proximal to the conical tip, wherein the infusion needle comprises an echogenic coating on at least a portion of its outer surface;
(b) a sheath surrounding the infusion needle, wherein the sheath is adjustable in length to accommodate an echoendoscope having a working channel diameter of at least 2.5 mm, optionally at least 2.8 mm; and
(c) a handle attached to the sheath and having an integrated syringe pump,
wherein administering the therapeutically effective amount of the gene therapy composition comprises delivering the gene therapy composition through the infusion needle at a controlled flow rate and pressure by actuating the syringe pump.

17. The method of claim 15, wherein the infusion is at a controlled pressure and rate to avoid pancreatic duct injury.

18. The method of claim 15, wherein the therapeutically effective amount is a single dose of about 3.0×10¹³ vector genomes (VG) to about 5.0×10¹³ VG, preferably 4.0×10¹³ VG.

19. The method of claim 15, wherein the therapeutically effective amount is:
a single dose of about 1.0×10¹⁴ VG to about 2.0×10¹⁴ VG, preferably 1.2×10¹⁴ VG; or
a single dose of about 3.0×10¹⁴ VG to about 4.0×10¹⁴ VG, preferably 3.6×10¹⁴ VG.

20. The method of claim 15, wherein the therapeutically effective amount is administered via two injections into the body and/or tail of the pancreas, optionally two 1 mL injections.


SEQUENCE LISTING (13 sequences)

SEQ ID NO: 1 - DNA, 243 bp, synthetic construct
  source 1..243: mol_type = other DNA
atggccctgtggatgcgcctcctgcccctgctggcgctgctggccctctggggacctgacccagccgcagccaggagcaaacgtcacgatgaatttgagagacatgctgaagggacctttaccagtgatgtgagttcttatttggaaggccaagctgccaaggaattcattgcttggctggtgaaaggccgaggaaggcgagatttcccagaagaggtcgccattgttgaagaacttggctaa

SEQ ID NO: 2 - DNA, 2319 bp, synthetic construct
  source 1..2319: mol_type = other DNA
cagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagaccagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagaccagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctcagccctccaggacaggctgcatcagaagaggccatcaagcaggtaagttccaagggcctttgcgtcaggtgggctcaggattccagggtggctggaccccaggccccagctctgcagcagggaggacgtggctgggctcgtgaagcatttgggggtgagcccaggggccccaaggcagggcacctggccttcagcctgcctcagccctgcctgtctttcagatcactgtccttctgccgccaccatggccctgtggatgcgcctcctgcccctgctggcgctgctggccctctggggacctgacccagccgcagccaggagcaaacgtcacgatgaatttgagagacatgctgaagggacctttaccagtgatgtgagttcttatttggaaggccaagctgccaaggaattcattgcttggctggtgaaaggccgaggaaggcgagatttcccagaagaggtcgccattgttgaagaacttggctaagctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 3 - DNA, 4129 bp, synthetic construct
  source 1..4129: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctcttctgagacgaccggtcagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagaccagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagaccagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctcagccctccaggacaggctgcatcagaagaggccatcaagcaggtaagttccaagggcctttgcgtcaggtgggctcaggattccagggtggctggaccccaggccccagctctgcagcagggaggacgtggctgggctcgtgaagcatttgggggtgagcccaggggccccaaggcagggcacctggccttcagcctgcctcagccctgcctgtctttcagatcactgtccttctgccgccaccatggccctgtggatgcgcctcctgcccctgctggcgctgctggccctctggggacctgacccagccgcagccaggagcaaacgtcacgatgaatttgagagacatgctgaagggacctttaccagtgatgtgagttcttatttggaaggccaagctgccaaggaattcattgcttggctggtgaaaggccgaggaaggcgagatttcccagaagaggtcgccattgttgaagaacttggctaagctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaatagttggcatctttataaggatgtaaatgataatgcttcagtgacaaattgtacgtttttatgtattttgcaaagtgctgtcaaatacatttctttggttgtctaactcgtagaactctaatagaaaatcagaatatcaatgacaatttgacattatttttaatcttttcttttctaaatagttgaataatttagaggaccctgtcctttttgtcctaaaaaaagggacagatatttaagttctatttatttataaaatcttggactcttattctaatggttcattatttttatagagctgtaggcatggttctttatttaattttttaaagttatttttaatttttgtggatacagagtaggtatacatatttatggggtatatgagatattttgatataagtatacaacatatataatccctttatttaattttatcttccccccaatgatctaaaactatttgcttgtccttttatgtcttatagttaaattcagtcaccaaagttgaagttacttcttatttttgcatagctccagctctgatcttcatctcatgtttttgcctgagcctctgttttcatattacttggttctgggagcatactttaatagcccagtcaagaaaaatactagctgccctgtcacccacactcctcacctgctagtcaacagcaaatcaacacaacaaaaatgaaaataatagacattatgcatgctctctaggaactgtcaattgaactgtatttgctcatcattcctaccatctacaccaccaaaatcaaccaaatttatgaaaaaaaacagccccaacataaaattatacacagataaacaggctatgattggttttgggaaagaagtcacctttacctgatttaggcaactgtgaaatgactagagaatgaagaaaattagacatttacatcttgtcatagagtttgaagatagtgctggatctttctttttataagtaagatcaataaaaactccctcattatatgatgtccctatgactaccagttagacctttagaagtcagaaaaaatgtgtttcaattgagaaaaaagataactggagtttgtgtagtacttcccagattataaaatgcttggttgtgatgtgggtgttagtgtacgccctagatgaaaggctccagagaggctgagccctctgctaacaagtcctactgctaacaagtgataaagccagagctggaagtcacatctggactccaaacctgatgcttctcagcctgttgccccttttagagttcctttttaatttctgcttttatgacttgctagatttctacctaccacacacactcttaaatggataattctgccctaaggataagtgattaccatttggttcagaactagaactaatgaattttaaaaattatttctgtatgtccattttgaattttcttatgagaaatagtatttgcctagtgttttcatataaaatatcccatgataataccatttactagtcgtctcaactaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - DNA, 10129 bp, synthetic construct
  source 1..10129: mol_type = other DNA
tcgcgcgtttcggtgatgacggtgaaaacctctgacacatgcagctcccgcagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgtgtggagattctttcttctgtttgaagtaatcccaagcatttcaaaggaattttttttaagttttctcaattattattaagtgtcctgatttgtaagaaacactaaaaagttgctcatagactgataagccattgtttcttttgtgatagagatgctttagctatgtccacagttttaaaatcatttctttattgagactaaacacaacagtcatggtgtatttaaatggcaatttgtcatttataaacacctctttttaaaatttgaggtttggtttctttttgtagaggctaatagggatatgatagcatgtatttatttatttatttatcttatggtatggtagtaagaacccttaacatgagatctaccctgttatatttttaagtgtacgatccattattgttaactactggtacactgttgtatagcttactcatcttgctgtattaaaactttgtgcccattgattagtaacccctagtttcatcctcccccatccactgggaaccagcattatactctttgattctatgagtttgactactttagctaccttatataagtggtattatgtactgtttatctttttatgactgacttatccacttcctcatagtgcattcaaagtccaaccatgttgttgcctattgcagaatttccttcttttcaaggctgaataatattccagtaccacattttctttatccattaatttgttgattgatagacatttaggttggttttctacatcttgactatcatgaatagtgttgcaatgaacacaggagagctactatctcttagagatgatataatggtttttatcatcagaaaacacccactgatttctatgctaattttgttgactgggtggaataatagtacagctatatattcctcattttagatatatttgtatttctacatacaataaaaaagcagagtacttagtcatgttgaagaactttaaacttttagtattttcaatcttcaaaacaaggacaggtttatctttctctcaccactcaatctatatatgactcttgtgggcaaggccagtttttatcactggagcctttcccctttttattatgtacctctccctcacagcagagtcaggactttaactttacacaatactatggctctacatataaaatcttaaaaatacataaaaattaataaattctgtctagggtagtatattttccctggggttacagttactttcataataaaaattagagataagtcaaggactcatttattggaaagtgattttaggtaacatttctggaagaaaaatgtctatatcttaatagtcacttaatatatgatggattgtgttactcctcagttttcaatggcatatactaaaacatggccctctaaaaagggggcaaatgaaatgagaaactctctgaatgtttttctccctcacctgctgcttagaagctaattttctcttgatttctgttataatgattgctcttacccttaagtttcaaaataggagtcatataactttccttaaagctattgactgtctttttgtcctgttttattcaccatgagttatagtgtgacagttaattcttatgaaaattatatagagatggttaaatcatcagaaactgtaaacctggattgggaggggaagtggatttttaaatgatttcctgaccaagctaaaccagtatattaaatcctttgtactgttcctataaagaaaattgcaactgtccagcaactgaaacctgctgatgggtttggtatcattggtgatagctgacagtgggttgagattgtcttctgtgctttgacgcgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctcttctgagacgaccggtcagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagaccagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagaccagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctcagccctccaggacaggctgcatcagaagaggccatcaagcaggtaagttccaagggcctttgcgtcaggtgggctcaggattccagggtggctggaccccaggccccagctctgcagcagggaggacgtggctgggctcgtgaagcatttgggggtgagcccaggggccccaaggcagggcacctggccttcagcctgcctcagccctgcctgtctttcagatcactgtccttctgccgccaccatggccctgtggatgcgcctcctgcccctgctggcgctgctggccctctggggacctgacccagccgcagccaggagcaaacgtcacgatgaatttgagagacatgctgaagggacctttaccagtgatgtgagttcttatttggaaggccaagctgccaaggaattcattgcttggctggtgaaaggccgaggaaggcgagatttcccagaagaggtcgccattgttgaagaacttggctaagctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaatagttggcatctttataaggatgtaaatgataatgcttcagtgacaaattgtacgtttttatgtattttgcaaagtgctgtcaaatacatttctttggttgtctaactcgtagaactctaatagaaaatcagaatatcaatgacaatttgacattatttttaatcttttcttttctaaatagttgaataatttagaggaccctgtcctttttgtcctaaaaaaagggacagatatttaagttctatttatttataaaatcttggactcttattctaatggttcattatttttatagagctgtaggcatggttctttatttaattttttaaagttatttttaatttttgtggatacagagtaggtatacatatttatggggtatatgagatattttgatataagtatacaacatatataatccctttatttaattttatcttccccccaatgatctaaaactatttgcttgtccttttatgtcttatagttaaattcagtcaccaaagttgaagttacttcttatttttgcatagctccagctctgatcttcatctcatgtttttgcctgagcctctgttttcatattacttggttctgggagcatactttaatagcccagtcaagaaaaatactagctgccctgtcacccacactcctcacctgctagtcaacagcaaatcaacacaacaaaaatgaaaataatagacattatgcatgctctctaggaactgtcaattgaactgtatttgctcatcattcctaccatctacaccaccaaaatcaaccaaatttatgaaaaaaaacagccccaacataaaattatacacagataaacaggctatgattggttttgggaaagaagtcacctttacctgatttaggcaactgtgaaatgactagagaatgaagaaaattagacatttacatcttgtcatagagtttgaagatagtgctggatctttctttttataagtaagatcaataaaaactccctcattatatgatgtccctatgactaccagttagacctttagaagtcagaaaaaatgtgtttcaattgagaaaaaagataactggagtttgtgtagtacttcccagattataaaatgcttggttgtgatgtgggtgttagtgtacgccctagatgaaaggctccagagaggctgagccctctgctaacaagtcctactgctaacaagtgataaagccagagctggaagtcacatctggactccaaacctgatgcttctcagcctgttgccccttttagagttcctttttaatttctgcttttatgacttgctagatttctacctaccacacacactcttaaatggataattctgccctaaggataagtgattaccatttggttcagaactagaactaatgaattttaaaaattatttctgtatgtccattttgaattttcttatgagaaatagtatttgcctagtgttttcatataaaatatcccatgataataccatttactagtcgtctcaactaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggaagcttgtctgtcctatcttcaatctttccctgcctatggtggtggtacctttctgtttttaacctggctataaattaccagataaacccattcactgatttgtaactcctttcagtcatgctctaactgtaaatgaaggcttaaactgaagtagaacagttacaaggttttacttggcagaacatctaggtgtctaagaagattttgggtttctcccattgtgaagaatagagtttcactcttgttgggtgcaatggtgtgatcttggttcagagcaacctctgcctcctgggttcaagtgattctcatgcctcagcctcccaagtagctgggattacaggcatgcgccaccacacctggctaattttgtatgggtttcaccatattgtctcgaactcctgagtgatccacccgccttggcctcccaaagtgctgggattacaggcatgagccaccttgcccagcctaagaagaacacaatagtgggacttggatacttgaagagatttttggaaatgatgtatttttcttctctatattccttcccttaattaactctgtttgttagatgtgcaaatatttggaatgataactcttttctcaaaacttataatattttctttctccctttgattaaacttatgggcaaatactagaatcctaatctctcatggcactttctggaaaatttaaggcggttattttatatatgtaagcagggcctatgactatgatcttgactcatttttcaaaaatcttctatattttatttagttatttggtttcaaaaggcctgcacttaattttgggggattatttggaaaaacagcattgatatacagtgcaatttagatcttttcttgagatggtttcaattctggaatcttaaacatgaaagaaaaagtagccttagaatgattaacaaaatttagactaattagaatagaaagatctgaatagagcaatctctaaaaaattttgatctttttttctctttttcacaatcctgagaacaaaaaaaaattaaatttaaatgttaattagaagatatttaacttagatgtaaagtgagttaacctgattccaggattaatcaagtactagaattagtatcttatggcaaattatagaacctatccctttagaatattttcaaatctttttgaggatgtttaggaatagttttacaagaaattaagttaggagaggaaatctgttctggaggatttttagggttcccactagcatatctaatggtttctgaactattcagaatcagagaaaactcatttttcctgctttcactactctactgtatgccctgcaccatgcacaaacaatgaccaatgtaaaatctctcattttggagagcctggaatctaactgtcaagtggaactaataataataatatgtacgatcatagccatcatttattaaacttttattatatgcaaggcactgtttaatttcattagcttacctggtttacagagcagctctatgagatgagtgccatctttgcccctattttctggataaggattctgaaatgtggagatgtgaagtaaaattgcacaactgaagaatgagttacatgacttggctcaaatactggtcattgaactccagagcctgaatattcttaaccacttacatgatgcaagctcaccaaataaatagttctaatgtattgtgacagagtgacattgatattcatctattcatgtgactttgcttctgaagaagaaaggataacattctgaccagaggggtgaaaaacaacctgcatctgatcctgaggcataatactattaacacaattcttttgatatcggatgccgtgaccgacgagtgcagaggcgtgcaagcgagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagccagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 5 - protein, 80 aa, synthetic construct
MALWMRLLPLLALLALWGPDPAAARSKRHDEFERHAEGTFTSDVSSYLEGQAAKEFIAWLVKGRGRRDFPEEVAIVEELG

SEQ ID NO: 6 - DNA, 1029 bp, synthetic construct
  source 1..1029: mol_type = other DNA
cagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagaccagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagaccagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctc

SEQ ID NO: 7 - DNA, 111 bp, synthetic construct
  source 1..111: mol_type = other DNA
cacgatgaatttgagagacatgctgaagggacctttaccagtgatgtgagttcttatttggaaggccaagctgccaaggaattcattgcttggctggtgaaaggccgagga

SEQ ID NO: 8 - DNA, 39 bp, Homo sapiens
  source 1..39: mol_type = genomic DNA
gatttcccagaagaggtcgccattgttgaagaacttggc

SEQ ID NO: 9 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 10 - DNA, 208 bp, Bos taurus
  source 1..208: mol_type = genomic DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 11 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 12 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 13 - DNA, 72 bp, Homo sapiens
  source 1..72: mol_type = genomic DNA
atggccctgtggatgcgcctcctgcccctgctggcgctgctggccctctggggacctgacccagccgcagcc